(19) United States
(12) Patent Application Publication
(10) Pub. No.: US 2013/0109698 A1
(43) Pub. Date: May 2, 2013

(54) Small Molecule Inhibitors of Functions of the HIV-1 Matrix Protein

(71) Applicant: Simon Cocklin, Philadelphia, PA (US)

(72) Inventor: Simon Cocklin, Philadelphia, PA (US)

(21) Appl. No.: 13/642,771

(22) PCT Filed: Apr. 25, 2011
(86) PCT No.: PCT/US11/33787

§ 371 Date: Jan. 18, 2013

Related U.S. Application Data
(60) Provisional application No. 61/330,743, filed on May 3, 2010.

(51) Int. Cl. A61K 31/496; A61K 31/40; C07D 491/056; A61K 31/454; C07D 271/06; C07D 295/195; A61K 31/495; C07D 405/14; A61K 31/4184; C07D 413/14; C07D 239/74; A61K 31/517; C07D 263/56; C07D 495/04; A61K 31/519; C07D 295/185; C07D 239/90; A61K 31/505; C07D 339/08; A61K 31/4025; C07C 237/10; A61K 31/165; A61K 31/4453; C07D 513/04; A61K 45/06; C07D 207/08

(52) U.S. Cl. CPC A61K 31/496; C07D 207/08; A61K 31/40; C07D 491/056; A61K 31/454; C07D 271/06; C07D 295/195; A61K 31/495; C07D 405/14; A61K 31/4184; C07D 413/14; C07D 239/74; A61K 31/517; C07D 263/56; C07D 495/04; A61K 31/519; C07D 295/185; C07D 239/90; A61K 31/505; C07D 339/08; A61K 31/4025; C07C 237/10; A61K 31/165; A61K 31/4453; C07D 513/04; A61K 45/06
USPC 514/252.17; 514/423; 514/322; 514/254.03; 514/255.01; 514/394; 514/321; 514/254.02; 514/260.1; 514/254.01; 514/266.3; 514/422; 514/616; 514/330

(57) ABSTRACT

The present invention includes a method of inhibiting, suppressing or preventing retroviral infection in a subject in need thereof, comprising administering to the subject a pharmaceutical composition comprising one or more of the compounds of the invention.

### STATEMENT REGARDING FEDERALLY SPONSORED RESEARCH OR DEVELOPMENT

This invention was made with government support under Grant Numbers 1R03AI078790-01A1 and 1R21AI087388-01A1 awarded by NIH/NIAID. The government has certain rights in the invention.

### BACKGROUND OF THE INVENTION

Despite recent progress in anti-HIV therapy, drug toxicity and the emergence of drug-resistant virus during long-term treatment of HIV-infected patients necessitate the search for new targets that may be used to develop novel antiviral agents, HIV-1 replication is a dynamic process influenced by a combination of viral and host factors (Freed, 2004, Trends Microbiol, 12(4):170-177). The full extent of the viral-host cell dependence is only now being realized (Brass et al., 2008, Science 319(5865):921-926; Konig et al., 2008, Cell 135(1):49-60; Zhou et al., 2008, Cell Host Microbe 4(5):495-504). In theory, all of the interactions between host cell and viral proteins could represent therapeutic intervention points in the HIV-1 replication cycle. The more stages of the viral life cycle that may be antagonized, the more likely it is that the virus would be forced to mutate at the expense of its fitness and the less likely that enough viral replication would occur to generate escape mutants.

The HIV-1 life cycle may be viewed as a sequence of steps that are regulated by both viral and cellular proteins. The first of the steps is attachment to the host cell and fusion of the viral and host membranes, Attachment and fusion of the virus to the host membrane are mediated by the viral envelope spike, which consists of a heterotrimer of 2 proteins; glycoprotein 120 (gp120) and glycoprotein 41 (gp41). During infection, gp120 binds to human cells expressing the receptor CD4, and to a second chemokine receptor, usually CCR5 or CXCR4 (Lusso, 2006, EMBO J. 25(3):447-456; Oppermann, 2004, Cell Signal 16(11): 1201-1210; Trkola et al., 1996, Nature 384(6605): 184-187). Binding to these receptors leads to conformational rearrangements within the viral envelope spike that ultimately result in the apposition of host and viral membranes, leading to fusion and entry of the virus into the host cell (Este & Telenti, 2007, Lancet 370(9581):81-88; Leonard & Roy, 2006, Curr. Med. Chem. 13(8):911-934; Pohlmann & Reeves, 2006, Curr. Pharm. Des. 12(16):1963-1973).

After fusion of viral envelope with the membrane of the host cell, the viral core is released into the cytosol, where it disassembles. Core disassembly is characterized by dissociation of capsid protein (CA), which forms the core shell (Auewarakul et al., 2005, Virology 337(1):93-101; McDonald et al., 2002, J. Cell. Biol. 159(3):441-452; Miller et al., 1997, J. Virol. 71(7):5382-5390), and the release of the reverse transcription complex (RTC) into the cytosol (Freed, 1998, Virology 251(1):1-15; Narayan & Young, 2004, Proc. Natl. Acad. Sci. U.S.A. 101(20):7721-7726). The RTC comprises genomic viral RNA associated with nucleocapsid protein (NC), cellular tRNA primer, enzymes reverse transcriptase, integrase, and protease, viral protein R (Vpr), and the matrix protein p17 (Briggs et al., 2003, EMBO J. 22(7):1707-1715; Bukrinskaya et al., 1992, AIDS Res, Hum. Retroviruses 8(10):1795-1801; Fassati & Goff, 2001, J. Virol. 75(8):3626-3635). The RTC then migrates towards the nucleus along the microtubule network, whereupon it reduces in size to facilitate entry through a nuclear pore. At this stage it becomes integration-competent to form what is termed the pre-integration complex (McDonald et al., 2002, J. Cell. Biol. 159(3):441-452; Miller et al., 1997, J. Virol. 71(7):5382-5390; Turelli et al., 2001, Mol. Cell. 7(6): 1245-1254).

Depending on the cell type, virus assembly takes place at the plasma membrane (Freed, 1998, Virology 251(1):1-15; Gottlinger, 2001, Aids 15 Suppl 5:S13-20) or in endosomal vacuoles (Ono & Freed, 2004, J. Virol. 78(3):1552-1563; Pelchen-Matthews et al., 2003, J. Cell. Biol. 162(3):443-455). Assembly is a multistep process in which the Gag polyprotein Pr55 plays the central role. Assembly is a multistep process and can be artificially divided into the following steps: (1) Gag dimerization and multimerization; (2) binding of Gag complexes to genomic viral RNA; and (3) transport of Gag/RNA complexes, Gag/Pol, Gag Pr55, and the viral envelope to the site of assembly. The final step of assembly involves a set of large assembly complexes comprising viral and cellular components (Freed, 2004, Trends Microbiol. 12(4):170-177; Alfadhli et al., 2005, J. Virol. 79(23):14498-14506; Halwani et al., 2003, J. Virol. 77(7):3973-3984). Virus particles are initially released as immature particles containing a spherical shell of structural proteins underneath the virus membrane. Virions subsequently undergo a maturation step that leads to the condensation of the inner core, formation of the core shell, and conversion of the virus particle into an infectious virion. The mature core is ready for disassembly in the infected cell, although the structural principles governing particle maturation remain elusive (Bukrinskaya, 2004, Arch. Virol. 149(6):1067-1082).

HIV-1 matrix Protein (MA) is a multifunctional protein with numerous and complex functions that is responsible for regulation of early and late steps of virus replication. The importance of MA in HIV-1 infection and its potential as an antiviral target have been recognized, leading to attempts to block its function using intrabodies or small molecules (Levin et al., 1997, Mol. Med. 3(2):96-110; Haffar et al., 2005, Expert Rev. AntiInfect. Ther. 3(1):41-50; Haffar et al., 2005, J, Virol. 79(20):13028-13036; Haffar et al., 1998, Antimicrob. Agents Chemother. 42(5): 1133-1138).

The HIV-1 matrix protein (MA) forms the N-terminal domain of the Pr55 Gag. MA is a 132-amino-acid structural protein that is post-translationally myristoylated at the N-terminus. The three-dimensional structure of HIV-1 MA has been determined by nuclear magnetic resonance (NMR) spectroscopy and x-ray crystallography (Hill et al., 1996, Proc. Natl. Assoc. Sci. U.S.A. 93(7):3099-3104; Massiah et al., 1994, J. Mot. Biol. 244(2):198-223; Verli et al., 2007, J. Mol. Graph. Model. 26(1):62-68).

MA is a structural molecule, partially globular, and composed of five α-helices. The helices α1, α2, and α3 are organized around the central and buried α4 (Hill et al., 1996, Proc. Natl. Acad. Sci. U.S.A. 93(7):3099-3104), whereas α5 is projected from the packed helical bundle, making the C-terminal region of the protein distinct from its globular N-terminus. Current data, based on crystallographic experiments (Hill et al., 1996, Proc. Natl. Acad. Sci. U.S.A. 93(7):3099-3104), electron microscopic observations (Nermut et al., 1998, J. Struct. Biol. 123(2):143-149; Nermut et al., 1994, Virology 198(1):288-296) and docking calculations (Forster et al., 2000, J. Mol. Biol. 298(5):841-857) point to a trimeric organization of p17 monomers in the mature virion. Such organization appears to be also used by other HIV-1 structural proteins (Huseby et al., 2005, J. Biol. Chem. 280(18):17664-17670), in an organization defined by the Gag precursor. A more recent study has indicated, however, that although the matrix protein p17 oligomerizes in solution as a trimer and crystallizes as a trimer unit, myristoylated matrix p17 assembles on phosphatidylserine-cholesterol membranes as hexamer rings (Alfadhli et al., 2007, J. Virol. 81(3):1472-1478). This finding has been echoed in a further study looking at oligomerization of MA on phosphatidylcholine-cholesterol-PI(4,5)P₂, where MA was found to organize as hexamers, each one composed of a trimer or MA (Alfadhli et al., 2009, Virology, in press).

The major functions for HIV-1 matrix have been established as: membrane binding, envelope incorporation, nucleic acid binding, and cytoplasmic trafficking.

### Membrane Binding:

Membrane binding of HIV-1 Gag is mediated by two signals in the matrix p17 portion: the N-terminal myristic acid and the conserved basic region between amino acids 17 and 31 (Bryant & Ratner, 1990, Proc. Natl. Acad. Sci. U.S.A. 87(2):523-527; Gottlinger et al., 1989, Proc. Natl. Acad. Sci. U.S.A. 86(15):5781-5785; Zhou et al., 1994, J. Virol. 68(4):2556-2569). The myristate moiety is considered to be regulated by a mechanism termed a myristoyl switch, whereby the N-terminal myristate is sequestered in the MA globular domain, but a structural change exposes the myristate and enhances Gag membrane binding (Hermida-Matsumoto & Resh, 1999, J. Virol. 73(3):1902-1908; Ono & Freed, 1999, J. Virol. 73(5):4136-4144). The MA basic domain is involved in specific localization of Gag to the plasma membrane, with mutations in this domain shifting the Gag localization from plasma membrane to intracellular vesicles in HeLa and T cells (Ono & Freed, 2004, J. Virol. 78(3):1552-1563; Freed et al., 1994, J. Virol. 68(8):5311-5320; Hermida-Matsumoto & Resh, 2000, J. Virol. 74(18):8670-8679; Ono et al., 2000, J. Virol. 74(6):2855-2866; Yuan et al., 1993, J. Virol. 67(11):6387-6394). This specific localization of Gag to the plasma membrane or related compartments suggests that host cellular factors play an important role in determining the site of virus assembly. Based on the similarity of the basic domain of MA with the membrane targeting sequences in other cellular proteins, it was proposed that Gag may interact with negatively charged lipids collectively known as phosphoinositides (DiNitto et al., 2003, Sci STKE 2003(213):re16; Hurley & Meyer, 2001, Curr. Opin. Cell Biol. 13(2):146-152; Lemmon, 2003, Traffic 4(4):201-213; Lietzke et al., 2000, Mol. Cell. 6(2):385-394). Different phosphoinositides localize in different subcellular membranes, thereby influencing the location of proteins to which they bind (Simonsen et al., 2001, Curr. Opin. Cell Biol. 13(4):485-492; Wenk & De Camilli, 2004, Proc. Natl. Acad. Sci. U.S.A. 101(22):8262-8269). Phosphatidylinositol (4,5)-bisphosphate [PI(4,5)P₂] is concentrated primarily on the cytoplasmic leaflet of the plasma membrane, which is also the site where Gag assembles in most cell types. Recently, three studies addressed the question of whether Gag interacts with PI(4,5)P₂ (Saad et al., 2006, Proc. Natl. Acad. Sci. U.S.A. 103(30): 11364-11369; Chukkapalli et al., 2008, J. Virol. 82(5):2405-2417; Shkriabai et al., 2006, Biochemistry 45(13):4077-4083). One study examined the accessibility of lysines after Gag is mixed with PI(4,5)P₂ (Shkriabai et al., 2006, Biochemistry 45(13):4077-4083). Two lysines were identified at MA, residues 29 and 31, as potential PI(4,5)P₂-interacting amino acids (Shkriabai et al., 2006, Biochemistry 45(13):4077-4083). The other group determined the structure of a complex between MA and PI(4,5)P₂ using NMR (Saad et al., 2006, Proc. Natl. Acad. Sci. U.S.A. 103(30):11364-11369). The third and most recent study monitored the overall membrane-binding ability of Gag in control and in PI(4,5)P₂-depleted cells using microscopy and an equilibrium flotation assay, in addition to looking at the Gag PI(4,5)P₂ interaction with an in vitro liposome-binding assay (Chukkapalli et al., 2008, J. Virol. 82(5):2405-2417). Altogether, results from these studies are consistent with the model that Gag binds PI(4,5)P₂ directly during virus assembly and that this interaction is mediated by the basic amino acids in the MA portion of Gag.

### Envelope Incorporation:

HIV-1 MA acts as a scaffold that brings together Env and Gag proteins in infected cells. It associates with the gp41 cytoplasmic tail during assembly and is responsible for incorporation of Env into virus particle (Bhattacharya et al., 2006, J. Virol. 80(11):5292-5300; Cosson, 1996, EMBO J. 15(21):5783-5788; Davis et al., 2006, J. Virol, 80(5):2405-2417; Dorfman et al., 1994, J. Virol. 68(3):1689-1696; Murakami & Freed, 2000, Proc. Natl. Acad. Sci. U.S.A. 97(1):343-348; Yu et al., 1993, J. Virol. 67(1):213-221). Until recently, this interaction was thought to be direct (Bhattacharya et al., 2006, J. Virol. 80(11):5292-5300; Wyma et al., 2000, J. Virol. 74(20):9381-9387). Lopez-Verges et al. (Proc. Natl. Acad. Sci. U.S.A. 2006, 103(40): 14947-14952), however, demonstrated that this association between the two viral structural proteins is mediated by a cellular factor—Tail Interacting Protein of 47 kDa (TIP47), a protein that normally functions in the retrograde transport of mannose 6-phosphate receptors from endosomes to the trans-Golgi network. TIP47 has the ability to interact with both the gp41 cytoplasmic domain and the MA domain of Gag. Disruption of either of these interactions leads to reduced Env incorporation and subsequent reduced infectivity of the budded viral particles. Abolition of these interactions by depletion of TIP47 also severely hinders the cell-to-cell spread of HIV-1 in culture (Lopez-Verges et al., 2006, Proc. Natl. Acad. Sci, U.S.A. 103(40): 14947-14952).

### Nucleic Acid Binding:

Although the nucleocapsid region of Gag is the predominant nucleic acid binding protein in the virus, MA also possesses a nucleic acid-binding region in its basic domain. This domain may bind genomic viral RNA as well as non-viral mRNAs and tRNAs, even in the absence of NC (nucloecapsid). More importantly, Gag without the MA basic region has a severe assembly defect (Ott et al., 2005, J. Virol. 79(22):13839-13847; Burniston et al., 1999, J. Virol. 73(10):8527-8540; Muriaux et al., 2004, Curr. Pharm. Des. 10(30):3725-3739). Recently, this interaction of the MA with RNA and DNA has been demonstrated biochemically using electrophoretic mobility shift assays (Hearps et al., 2008, Biochemistry 47(7):2199-2210). Hearps and co-workers demonstrated that residues 2-33 imparted this nucleic acid binding ability, and mutation of residues K26 and K27 to threonine completely abolished this capability, probably by disrupting the protein's overall structure (Biochemistry 2008, 47(7):2199-2210).

### Cyloplasmic Trafficking:

Comparatively, very little is known about the cytoplasmic trafficking of the Gag protein to its final destination at the plasma membrane as compared with its other functions, although studies are underway. A recent report has identified the suppressor of cytokine signaling 1 (SOCS1) as host cell factor, which is induced upon infection of a host cell by HIV-1 (Ryo et al., 2008, Proc. Natl. Acad. Sci. U.S.A. 105(1):294-299). The depletion of SOCS1 by small interfering RNA (siRNA) reduces both the targeted trafficking and assembly of HIV-1 Gag, resulting in its accumulation as perinuclear solid aggregates that are eventually subjected to lysosomal degradation. SOCS1 was subsequently found to be able to bind to the Gag polyprotein via regions including the N-terminus and its SH2 domain. Analysis of the SOCS1-binding region on Gag identified MA and NC regions as participating in this interaction. SOCS1 is known to be required for microtubule stability and also increases the amount of Gag associated with the microtubule network in a ubiquitin-dependent manner. Another study has implicated the Golgi-localized γ-ear-containing Arf-binding (GGA) and adenosine diphosphate ribosylation factor (Artf) proteins in retrovirus particle assembly and release (Joshi et al., 2008, Mol. Cell. 30(2):227-238). Depletion of GGA2 and GGA3 led to a significant increase in particle release in a late domain-dependent manner, whereas GGA overexpression severely reduced retrovirus particle production by impairing Gag trafficking to the membrane. GGA overexpression inhibited retroviral assembly and release by disrupting the endogenous Arf protein activity. Furthermore, disruption of Arf activity inhibited particle production by decreasing Gag-membrane binding (Joshi et al., 2008, Mol. Cell. 30(2):227-238). The effects of Arf disruption, either by GGA sequestration, mutation, or knockdown, on the ability of Gag to bind to membrane may implicate an interaction of one or both with the MA domain of Gag. These potential interactions, however, have yet to be definitively demonstrated.

The HIV-1 matrix protein (MA) is thus involved in multiple stages in the retrovirus life cycle. HIV-1 MA has been implicated in novel roles during infection including virion assembly, viral entry/uncoating, cytoskeletal-mediated transport, and targeting viral assembly to lipid rafts, but details about these functions are still emerging. There remains a need in the art to identify novel small-molecule inhibitors of retroviral MA activity, including HIV-1 MA activity. These inhibitors may be used to interfere with one or more biological functions of retroviral MA and lead to impairment of retrovirus life cycle and infection. The present invention fulfills these needs.

### BRIEF SUMMARY OF THE INVENTION

The invention includes a method of inhibiting, suppressing or preventing a retrovirus infection in a subject in need thereof. The method comprises administering to the subject a composition comprising a therapeutically effective amount of at least one compound selected from the group consisting of:
N-benzyl-3-(2-(2-(2,5-dimethoxyphenyl)pyrrolidin-1-yl)-2-oxoethoxy)benzamide (CMPD-1);
2-(2-methyl-6,7-dihydro-1H-[1,4]dioxino[2′,3′:4,5]benzo[1,2-d]imidazol-1-yl)-N-(3-(piperidin-1-ylsulfonyl)phenyl)acetamide (CMPD-2);
(3,5-dichloro-4-methoxyphenyl)(4-((3-(p-tolyl)-1,2,4-oxadiazol-5-yl)methyl)piperazin-1-yl)methanone (CMPD-3);
N-(4-(cyclopropanecarboxamido)benzyl)-4-(2-(4-methoxyphenoxy)ethyl)piperazine-1-carboxamide (CMPD-4);
1-(2,3-dihydrobenzo[b][1,4]dioxin-6-yl)-N-((1-methyl-1H-benzo[d]imidazol-2-yl)methyl)-5-oxopyrrolidine-3-carboxamide (CMPD-5);
2-(4-(3-(benzo[d]oxazol-2-yl)propanoyl)piperazin-1-yl)-N-(4-(trifluoromethoxy)phenyl)acetamide (CMPD-9);
N-(5-acetamido-2-methoxyphenyl)-2-(5-(5-methylthiophen-2-yl)-4-oxothieno[2,3-d]pyrimidin-3(4H)-yl)acetamide (CMPD-10);
N-(benzylcarbamoyl)-2-(4-(2-oxo-2-(pyrrolidin-1-yl)ethyl)piperazin-1-yl)acetamide (CMPD-11);
2-(2-oxo-2-((4-(pyrrolidin-1-yl)phenyl)amino)ethoxy)-N-phenylbenzamide (CMPD-115);
N-(4-((4-bromophenyl)thio)phenyl)-2-(4-oxoquinazolin-3(4H)-yl)acetamide (CMPD-16);
4-(1,3-dithian-2-yl)-N-(4-(pyrrolidin-1-ylmethyl)benzyl)benzamide (CMPD-19);
2-acetamido-N-(4-(piperidine-1-carbonyl)benzyl)acetamide (CMPD-45);
   a compound of Formula (I);
   a compound of Formula (II);
   a compound of Formula (III);
   a compound of Formula (IV);
   a compound of Formula (V);
   a compound of Formula (VI);
   a compound of Formula (VII);
   a compound of Formula (VIII);
      mixtures thereof and pharmaceutically acceptable carriers thereof, wherein:
      the compound of Formula (I) is:

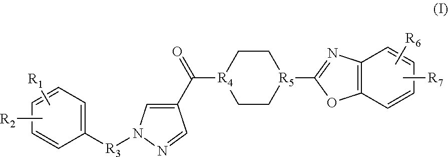

wherein in Formula (I):
   R₁, R₂, R₆ and R₇ are independently H, fluoro, chloro, bromo, C₁-C₆ alkyl, or C₁-C₆ alkoxy,
   R₃ is CH₂ or a chemical bond, and
   R₄ and R₅ are independently CH or N,
   or a pharmaceutically acceptable salt thereof;
      the compound of Formula (I) is:
wherein in Formula (II):

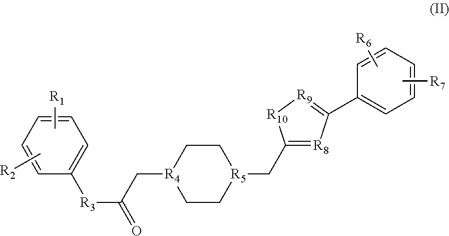

R₁, R₂, R₆ and R₇ are independently H, fluoro, chloro, bromo, —CF₃, C₁-C₆ alkyl, or C₁-C₆ alkoxy,
R₃ is NH, CH₂ or a chemical bond,
R₄ and R₅ are independently CH or N,
R₈ and R₉ are independently CH or N, and
R₁₀ is O or S;
or a pharmaceutically acceptable salt thereof;
   the compound of Formula (III) is:

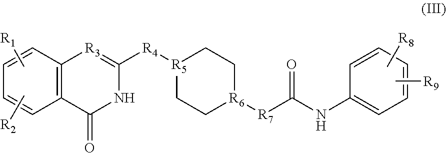

wherein in Formula (II):
   R₁, R₂, R₈ and R₉ are independently H, fluoro, chloro, bromo, C₁-C₆ alkyl, or C₁-C₆ alkoxy,
   R₃ is CH or N,
   R₄ and R₇ are independently CH₂ or a chemical bond, and
   R₅ and R₆ are independently CH or N;
   or a pharmaceutically acceptable salt thereof;
      the compound of Formula (IV) is:

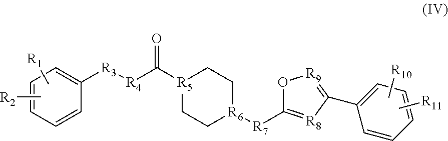

wherein in Formula (LV):
   R₁, R₂, R₁₀ and R₁₁ are independently H, fluoro, chloro, bromo, C₁-C₆ alkyl, or C₁-C₆ alkoxy,
   R₃ is O or NH,
   R₄ and R₇ are independently CH₂ or a chemical bond, and
   R₅, R₆, R₈ and R₉ are independently CH or N;
   or a pharmaceutically acceptable salt thereof;
      the compound of Formula (V) is:

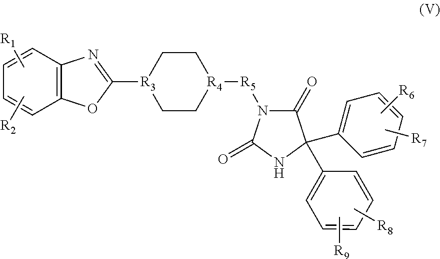

wherein in Formula (V):
   R₁, R₂, R₆, R₇, R₈ and R₉ are independently H, fluoro, chloro, bromo, C₁-C₆ alkyl, or C₁-C₆ alkoxy,
   R₃ are R₄ are independently CH or N, and
   R₅ is CH₂ or a chemical bond;
   or a pharmaceutically acceptable salt thereof;
      the compound of Formula (VI) is:
wherein in Formula (VI):

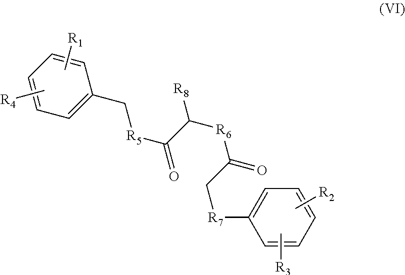

R₁, R₂, and R₃ are independently H, fluoro, chloro, bromo, C₁-C₆ alkyl, or C₁-C₆ alkoxy,
R₄ is H, halo, C₁-C₆ alkyl, C₁-C₆ alkoxy, —SO₂NH₂, —NH₂, —NHC(═O)CH₃, —CF₃, or —C(═O)R₉,
R₅, R₆ and R₇ are independently a chemical bond, O, CH₂ or NH,
R₈ is H or C₁-C₆ alkyl,
R₉ is H, C₁-C₆ alkyl, N,N-dimethylamino, N,N-diethylamino, N-morpholinyl, N-pyrrolidinyl, N-piperidinyl, N-piperazinyl, or N′—(C₁-C₆ alkyl)piperazinyl,
or a pharmaceutically acceptable salt thereof;
   the compound of Formula (VII) is:

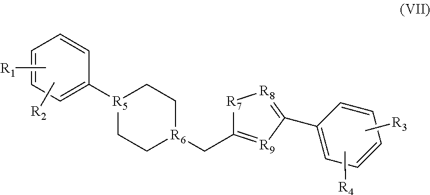

wherein in Formula (VII):
   R₁, R₂, R₃, and R₄ are independently H, nitro, amino, fluoro, chloro, bromo, C₁-C₆ alkyl, or C₁-C₆ alkoxy,
   R₅, R₆, R₈ and R₉ are independently CH or N, and
   R₇ is S or O,
   or a pharmaceutically acceptable salt thereof;
      and the compound of Formula (VIII) is:

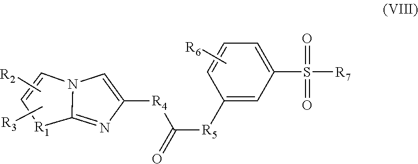

wherein in Formula (VIII):
   R₁ is O, S or —CH═CH—;
   R₂, R₃, and R₆ are independently H, fluoro, chloro, bromo, C₁-C₆ alkyl, or C₁-C₆ alkoxy,
   R₄ and R₅ are independently CH₂ or NH, and
   R₇ is C₁-C₆ alkyl, N,N-dimethylamino, N,N-diethylamino, N-morpholinyl, N-pyrrolidinyl or N-piperidinyl,
   or a pharmaceutically acceptable salt thereof.

In one embodiment, the at least one compound is selected from the group consisting of:
N-benzyl-3-(2-(2-(2,5-dimethoxyphenyl)pyrrolidin-1-yl)-2-oxoethoxy)benzamide (CMPD-1);
2-(2-methyl-6,7-dihydro-1H-[1,4]dioxino[2′,3′:4,5]benzo[1,2-d]imidazol-1-yl)-N-(3-(piperidin-1-ylsulfonyl)phenyl)acetamide (CMPD-2);
(3,5-dichloro-4-methoxyphenyl)(4-((3-(p-tolyl)-1,2,4-oxadiazol-5-yl)methyl)piperazin-1-yl)methanone (CMPD-3);
N-(4-(cyclopropanecarboxamido)benzyl)-4-(2-(4-methoxyphenoxy)ethyl)piperazine-1-carboxamide (CMPD-4);
1-(2,3-dihydrobenzo[b][1,4]dioxin-6-yl)-N-((1-methyl-1H-benzo[d]imidazol-2-yl)methyl)-5-oxopyrrolidine-3-carboxamide (CMPD-5);
(4-(benzo[d]oxazol-2-yl)piperidin-1-yl)(1-(2-chlorobenzyl)-1H-pyrazol-4-yl)methanone (CMPD-6);
2-(4-((3-(4-fluorophenyl)-1,2,4-oxadiazol-5-yl)methyl)piperazin-1-yl)-N-(p-tolyl)acetamide (CMPD-7);
2-(4-((6,7-dimethoxy-4-oxo-3,4-dihydroquinazolin-2-yl)methyl)piperazin-1-yl)-N-(2,3-dimethylphenyl)acetamide (CMPD-8);
2-(4-(3-(benzo[d]oxazol-2-yl)propanoyl)piperazin-1-yl)-N-(4-(trifluoromethoxy)phenyl)acetamide (CMPD-9);
N-(5-acetamido-2-methoxyphenyl)-2-(5-(5-methylthiophen-2-yl)-4-oxothieno[2,3-d]pyrimidin-3(4H)-yl)acetamide (CMPD-10);
N-(benzylcarbamoyl)-2-(4-(2-oxo-2-(pyrrolidin-1-yl)ethyl)piperazin-1-yl)acetamide (CMPD-11);
2-(2,5-dichlorophenoxy)-1-(4-((3-(4-methoxyphenyl)-1,2,4-oxadiazol-5-yl)methyl)piperazin-1-yl)ethanone (CMPD-12); 3-((4-(benzo[d]oxazol-2-yl)piperidin-1-yl)methyl)-5,5-diphenyl imidazolidine-2,4-dione (CMPD-13);
N-(2-oxo-2-((4-(piperidine-1-carbonyl)benzyl)amino)ethyl)-2-phenoxyacetamide (CMPD-14);
2-(2-oxo-2-((4-(pyrrolidin-1-yl)phenyl)amino)ethoxy)-N-phenylbenzamide (CMPD-15);
N-(4-((4-bromophenyl)thio)phenyl)-2-(4-oxoquinazolin-3(4H)-yl)acetamide (CMPD-16);
3-(2-ethoxyphenyl)-5-((4-(4-(4-nitrophenyl)piperazin-1-yl)methyl)-1,2,4-oxadiazole (CMPD-17);
N-(4-ethoxy-3-(piperidin-1-ylsulfonyl)phenyl)-2-(imidazo[2,1-b]thiazol-6-yl)acetamide (CMPD-18);
4-(1,3-dithian-2-yl)-N-(4-(pyrrolidin-1-ylmethyl)benzyl)benzamide (CMPD-19);
N-(4-fluorophenyl)-2-(4-((3-(4-fluorophenyl)-1,2,4-oxadiazol-5-yl)methyl)piperazin-1-yl)acetamide (CMPD-20);
N-(2,3-dimethylphenyl)-2-(4-((3-(4-fluorophenyl)-1,2,4-oxadiazol-5-yl)methyl)piperazin-1-yl)acetamide (CMPD-21);
N-(2-chlorophenyl)-2-(4-((3-(4-fluorophenyl)-1,2,4-oxadiazol-5-yl)methyl)piperazin-1-yl)acetamide (CMPD-22);
N-(2,6-dimethylphenyl)-2-(4-((3-(4-fluorophenyl)-1,2,4-oxadiazol-5-yl)methyl)piperazin-1-yl)acetamide (CMPD-23);
N-(3-fluorophenyl)-2-(4-((3-(4-fluorophenyl)-1,2,4-oxadiazol-5-yl)methyl)piperazin-1-yl)acetamide (CMPD-24);
N-(2-ethylphenyl)-2-(4-((3-(4-fluorophenyl)-1,2,4-oxadiazol-5-yl)methyl)piperazin-1-yl)acetamide (CMPD-25);
2-(4-((3-(4-fluorophenyl)-1,2,4-oxadiazol-5-yl)methyl)piperazin-1-yl)-N-(2-(trifluoromethyl)phenyl)acetamide (CMPD-26);
2-(2-phenoxyacetamido)-N-(3-(trifluoromethyl)benzyl)propanamide (CMPD-27);
2-(2-phenoxyacetamido)-N-(4-sulfamoylbenzyl)propanamide (CMPD-28), 2-(2-phenoxyacetamido)-N-(4-methoxybenzyl)propanamide (CMPD-29);
3-methyl-N-(4-methylbenzyl)-2-(2-phenoxyacetamido)butanamide (CMPD-30);
N-benzyl-3-methyl-2-(2-phenoxyacetamido)butanamide (CMPD-32);
N-(4-acetamidobenzyl)-2-(2-phenoxyacetamido)propanamide (CMPD-33);
N-(3-acetamidobenzyl)-2-(2-phenoxyacetamido)acetamide (CMPD-34);
N-(4-chlorobenzyl)-2-(2-phenoxyacetamido)acetamide (CMPD-35);
N-(4-methoxybenzyl)-2-(2-phenoxyacetamido)acetamide (CMPD-36);
N-(2-oxo-2-((4-(trifluoromethyl)benzyl)amino)ethyl)-2-phenoxyacetamide (CMPD-37);
N-benzyl-2-(2-phenoxyacetamido)acetamide (CMPD-38);
N-(3-chlorobenzyl)-2-(2-phenoxyacetamido)acetamide (CMPD-39);
N-(4-(4-methylpiperazine-1-carbonyl)benzyl)-2-(2-phenoxyacetamido) acetamide (CMPD-40);
2-acetamido-N-(4-(4-methylpiperazine-1-carbonyl)benzyl)acetamide (CMPD-41);
2-(4-chlorophenoxy)-N-(2-((4-(4-methylpiperazine-1-carbonyl)benzyl)amino)-2-oxoethyl)acetamide (CMPD-42);
2-(4-methoxyphenoxy)-N-(2-((4-(4-methylpiperazine-1-carbonyl)benzyl)amino)-2-oxoethyl)acetamide (CMPD-43);
N-(4-(4-methylpiperazine-1-carbonyl)benzyl)-2-(2-(4-(trifluoromethyl)phenoxy)acetamido)acetamide (CMPD-44);
2-acetamido-N-(4-(piperidine-1-carbonyl)benzyl)acetamide (CMPD-45); 2-(4-chlorophenoxy)-N-(2-oxo-2-((4-(piperidine-1-carbonyl)benzyl)amino)ethyl)acetamide (CMPD-46);
2-(4-methoxyphenoxy)-N-(2-oxo-2-((4-(piperidine-1-carbonyl)benzyl)amino)ethyl)acetamide (CMPD-47);
N-(2-oxo-2-((4-(piperidine-1-carbonyl)benzyl)amino)ethyl)-2-(4-(trifluoromethyl)phenoxy)acetamide (CMPD-48);
3-(4-fluorophenyl)-5-((4-(2-methoxyphenyl)piperazin-1-yl)methyl)-1,2,4-oxadiazole (CMPD-49);
3-(4-fluorophenyl)-5-((4-(4-methoxyphenyl)piperazin-1-yl)methyl)-1,2,4-oxadiazole (CMPD-50);
3-(2-ethoxyphenyl)-5-((4-(4-nitrophenyl)piperazin-1-yl)methyl)-1,2,4-oxadiazole (CMPD-51);
3-(4-fluorophenyl)-5-((4-phenylpiperazin-1-yl)methyl)-1,2,4-oxadiazole (CMPD-52);
   a mixture thereof and a pharmaceutically acceptable carrier thereof.

In one embodiment, the at least one compound is selected from the group consisting of:
(4-(benzo[d]oxazol-2-yl)piperidin-1-yl)(1-(2-chlorobenzyl)-1H-pyrazol-4-yl)methanone (CMPD-6);
2-(4-((3-(4-fluorophenyl)-1,2,4-oxadiazol-5-yl)methyl)piperazin-1-yl)-N-(p-tolyl)acetamide (CMPD-7);
2-(4-((6,7-dimethoxy-4-oxo-3,4-dihydroquinazolin-2-yl)methyl)piperazin-1-yl)-N-(2,3-dimethylphenyl)acetamide (CMPD-8);
2-(2,5-dichlorophenoxy)-1-(4-((3-(4-methoxyphenyl)-1,2,4-oxadiazol-5-yl)methyl)piperazin-1-yl)ethanone (CMPD-12);
3-((4-(benzo[d]oxazol-2-yl)piperidin-1-yl)methyl)-5,5-diphenylimidazolidine-2,4-dione (CMPD-13);
N-(2-oxo-2-((4-(piperidine-1-carbonyl)benzyl)amino)ethyl)-2-phenoxyacetamide (CMPD-14);
3-(2-ethoxyphenyl)-5-((4-(4-nitrophenyl)piperazin-1-yl)methyl)-1,2,4-oxadiazole (CMPD-17);
N-(4-ethoxy-3-(piperidin-1-ylsulfonyl)phenyl)-2-(imidazo[2,1-b]-thiazol-6-yl)acetamide (CMPD-18);
   a mixture thereof and a pharmaceutically acceptable carrier thereof.

In one embodiment, the at least one compound is (4-(benzo[d]oxazol-2-yl)piperidin-1-yl)(1-(2-chlorobenzyl)-1H-pyrazol-4-yl)methanone (CMPD-6), or a pharmaceutically acceptable carrier thereof. In another embodiment, the at least one compound is 2-(4-((3-(4-fluorophenyl)-1,2,4-oxadiazol-5-yl)methyl)piperazin-1-yl)-N-(p-tolyl)acetamide (CMPD-7), or a pharmaceutically acceptable carrier thereof. In yet another embodiment, the at least one compound is 2-(4-((6,7-dimethoxy-4-oxo-3,4-dihydroquinazolin-2-yl)methyl)piperazin-1-yl)-N-(2,3-dimethylphenyl)acetamide (CMPD-8), or a pharmaceutically acceptable carrier thereof. In yet another embodiment, the at least one compound is 2-(2,5-dichlorophenoxy)-1-(4-((3-(4-methoxyphenyl)-1,2,4-oxadiazol-5-yl)methyl)piperazin-1-yl)ethanone (CMPD-12), or a pharmaceutically acceptable carrier thereof. In yet another embodiment, the at least one compound is 3-((4-(benzo[d]oxazol-2-yl)piperidin-1-yl)methyl)-5,5-diphenylimidazolidine-2,4-dione (CMPD-13), or a pharmaceutically acceptable carrier thereof. In yet another embodiment, the at least one compound is N-(2-oxo-2-((4-(piperidine-1-carbonyl)benzyl)amino)ethyl)-2-phenoxyacetamide (CMPD-14), or a pharmaceutically acceptable carrier thereof. In yet another embodiment, the at least one compound is 3-(2-ethoxyphenyl)-5-((4-(4-nitrophenyl)piperazin-1-yl)methyl)-1,2,4-oxadiazole (CMPD-17), or a pharmaceutically acceptable carrier thereof. In yet another embodiment, the at least one compound is N-(4-ethoxy-3-(piperidin-1-ylsulfonyl)phenyl)-2-(imidazo[2,1-b]thiazol-6-yl)acetamide (CMPD-18), or a pharmaceutically acceptable carrier thereof.

In one embodiment, the retrovirus is selected from the group consisting of ALV, RSV, MMTV, SRV, HERV-K, JRSV, MLV, FeLV, GALV, PERV, HERV-W, BLV, HTLV-I, HTLV-II, WDSV, SnRV, HIV-1, HIV-2, SIVmac, SIV, FIV, EIAV, MVV, SFVcpz, SFVagm, FFV, BFV and combinations thereof. In another embodiment, the retrovirus is selected from the group consisting of MLV, HIV-1, SIV, and combinations thereof.

In one embodiment, the composition further comprises one or more anti-HIV drugs. In another embodiment, the one or more anti-HIV drugs are selected from the group consisting of combination drugs, entry and fusion inhibitors, integrase inhibitors, non-nucleoside reverse transcriptase inhibitors, nucleoside reverse transcriptase inhibitors, and protease inhibitors.

In one embodiment, the subject is a mammal. In another embodiment, the subject is human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic representation of the domain architecture of the Gag Pr55 polyprotein. Legend: MA is matrix p17, CA is capsid, NC is nucleocapsid, SP1 is spacer peptide 1, and SP2 is spacer peptide 2.

FIG. 2 is a ribbon representation of the HIV-1 matrix protein p17, with the individual helices that form the structure shown.

FIG. 3, comprising FIGS. 3A-3C, is a series of schematic representations of the HIV-1 MA. FIG. 3A is a schematic representation of HIV-1 MA displaying the positions of known functional domains. FIG. 3B is an expanded view of the basic domain and individual amino acids and their ascribed functions.

FIG. 3C is a ribbon diagram of the HIV-1 MA depicting the positions of the functional domain.

FIG. 4, comprising FIGS. 4A-4B, is a schematic representation of imposed docking area restriction on HIV-1 matrix, where the boxes restricting the docking area are shown. FIG. 4A is the ribbon diagram of x-ray structure 2GOL. FIG. 4B is ribbon diagram of the NMR structure 2H3Z with di-C4-PI(4,5)P₂ bound. The protein is shown in the ribbon representation, whereas di-C4-PI(4,5)P₂ is shown in sphere representation.

FIG. 5, comprising FIGS. 5A-5B, illustrates the docking of IT-367 (FIG. 5A) and of the hits identified in this study (FIG. 5B) in HIV-1 MA as predicted by Quantum molecular modeling software. The proteins are illustrated as surface renderings and the compounds are illustrated in space filling representations.

FIG. 6 is a reproduction of a gel illustrating the subtractive purification of HIV-1_{LAI} MA protein. Lane 1 corresponds to Novex Sharp Pre-Stained Protein Standards (Invitrogen, Carlsbad, Calif.) (molecular weights in kDa). Lane 2 corresponds to whole cell lysate. Lane 3 corresponds to cleared cell lysate. Lane 4 corresponds to cobalt column flow-through. Lane 5 corresponds to 250 mM imidazole elution containing SUMO-MA fusion protein. Lane 6 corresponds to dtUD1-catalyzed cleavage reaction. Lane 7 corresponds to subtracted flow-through from cobalt column containing native MA. Lane 8 corresponds to 250 mM imidazole elution containing SUMO protein. Lane 9 corresponds to Novex Sharp Pre-Stained Protein Standards (Invitrogen, Carlsbad, Calif.).

FIG. 7 is a flow chart representation of the studies used to characterize compounds of the invention described herein.

FIG. 8 is a graphical representation of HIV-1 MA surface illustrating the residues that form the collective compound binding site.

FIG. 9, comprising FIGS. 9A-9B, illustrates the inhibition of HIV-1 infection by compounds of the invention. FIG. 9A illustrates the effect of compounds on production of infectious virus from 293T cells. FIG. 9B illustrates the effect of compounds on the infection of recombinant luciferase-containing HIV-1 viruses (HIV-1NL4-3 backbone) pseudotyped with the envelope protein from HIV-1HxBc2. The U87 cell lines stably expressing CD4 and CXCR4 were used as target cells.

FIG. 10 is a bar graph illustrating the inhibition of infection of SupT1 cells by HIV-1 IIIB. Sup-T1 cells (1×10⁵ cells in 1 mL) were infected with cell-free HIV-1 IIIB (10 μL of a 1:300 dilution of a 1×10⁷ tissue culture dose for 50% infectivity (TCID50) stock; Advanced Biotechnologies, Inc., Columbia, Md.) in the presence or absence of each compound (50 μM). After incubation for three days at 37° C., cell-free culture supernatants were assayed for the presence of HIV-1 by p24 ELISA (Zeptometrix Corp., Buffalo, N.Y.).

FIG. 11 is a graph illustrating the qualitative comparison of the interaction of compound CMPD-18 (25.1 μM) with sensor-chip-immobilized HIV-1 MA and SUMO proteins.

FIG. 12 is a figure illustrating the conservation level of surface residues of the HIV-1 MA protein. Legend: A=>99% conservative residue; B=>80%-99%; C=>50%-80%; D=>20%-50%; E=0%-20%. Residues surrounding a structural recess that serves as a binding site for PI(4,5)P₂ are highly conserved as indicated by the red coloring.

FIG. 13, comprising FIGS. 13A-13F, is a series of graphs illustrating the inhibition of HIV-1 infection by compounds of the invention. 293T cells were transfected for production of luciferase-reporter pseudotypes (as described in Materials and Methods) either in the absence or in the presence of compounds, and the culture supernatants containing pseudotype stocks were then diluted 10-fold and used to infect target U87.CD4.CXCR4 cells in the absence of compound, to determine their potential to affect late stages in the viral life cycle (FIGS. 13A-13C); compound-induced effects are manifested as a decrease in infectivity in the target cells (measured as luciferase activity), normalized against the infectivity of virus from untreated cells. Complementarily, effects on early-stage events were determined by using virus produced in the absence of compound for infection of target cells in the absence or presence of various concentrations of compounds (FIGS. 13D-13F). Data are shown as mean±standard error of the mean from at least four independent experiments (each performed in triplicate).

FIG. 14 is a bar graph illustrating direct binding of compounds of the invention to HIV-1 MA via surface plasmon resonance, MA protein was immobilized onto a CM7 sensor chip at 19,000 RU via standard amine coupling. The maximum response, after reference surface subtraction, for each compound at 10 μM is shown, along with a nonspecific small molecule (NBD-556).

FIG. 15, comprising FIGS. 15A-15H, illustrates the effects of compounds of the invention on production and infection processes involving HIV-MA point mutants. FIG. 15A illustrates the binding pocket residues within 4 Å of CMPD-18. FIG. 15B illustrates the production and infection inhibition of HIV-MA mutants by CMPD-18. FIG. 15C illustrates the binding pocket residues within 4 Å of CMPD-17. FIG. 15D illustrates the production and infection inhibition of HIV-MA mutants by CMPD-17. FIG. 15E illustrates the binding pocket residues within 4 Å of CMPD-7. FIG. 15F illustrates the production and infection inhibition of HIV-MA mutants by CMPD-7. FIG. 15G illustrates the binding pocket residues within 4 Å of CMPD-14. FIG. 15H illustrates the production and infection inhibition of HIV-MA mutants by CMPD-14.

FIG. 16 is a fluxogram illustrating a compound evaluation process.

FIG. 17 is a schematic representation of a HIV-1 single-round infection assay.

FIG. 18 is a schematic representation of a single-round infection assay with a late stage defect.

FIG. 19 is a schematic representation of a single-round infection assay with an early stage defect.

FIG. 20 is a graph illustrating the replication inhibition of a primary isolate in peripheral blood mononuclear cells (PBMC).

FIG. 21 is a graph illustrating the effect of compounds of the invention on the replication of herpes simplex virus 1 (HSV-1) (dsDNA virus).

FIG. 22 is bar graph illustrating the % viral infection of HIV-MA point mutants as compared to wild-type HIV-MA.

### DETAILED DESCRIPTION OF THE INVENTION

The present invention relates to the discovery that the compounds of the present invention may be used to inhibit, suppress or prevent a retroviral infection in a vertebrate cell. The compounds of the present invention bind to the retrovirus matrix protein and inhibit one or more of its biological functions, compromising the retrovirus life cycle.

In one aspect, the invention includes a method of inhibiting, suppressing or preventing retrovirus infection in a subject in need thereof. The method comprises the step of treating a subject with a therapeutically effective amount of a pharmaceutical composition comprising at least one compound of the invention. In one embodiment, the subject is human. In another embodiment, the retrovirus comprises at least one virus selected from the group consisting of HIV-1, SIV and MLV.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “PBMC” refers to a peripheral blood mononuclear cell (PBMC).

As used herein, the term “MTI” refers to a MA-targeted inhibitory compound or a salt thereof.

As used herein, the term “retrovirus” or “retroviral” refers to an RNA virus that is replicated in a host cell via the enzyme reverse transcriptase to produce DNA from its RNA genome. Examples of retroviruses include but are not limited to the following virus genera: Genus Alpharetrovirus [such as avian leukosis virus (ALV) and rous sarcoma virus (RSV)]; Genus Betaretrovirus [such as mouse mammary tumour virus (MMTV), SRV, HERV-K and JRSV]; Genus Gammaretrovirus [such as murine leukemia virus (MLV), feline leukemia virus (FeLV), GALV, PERV, and HERV-W]; Genus Deltaretrovirus [such as bovine leukemia virus (BLV), and cancer-causing human T-lymphotrophic virus (HTLV-I and HTLV-II)]; Genus Epsilonretrovirus [such as Walleye dermal sarcoma virus (WDSV) and SnRV]; Genus Lentivirus [such as human immunodeficiency virus 1 (HIV-1), human immunodeficiency virus 2 (HIV-2), simian immunodeficiency virus (SIVmac and SIV), feline immunodeficiency virus (FIV), EIAV and MVV]; and Genus Spumavirus [such as simian foamy virus (SFVcpz and SFVagm), FFV and BFV].

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, amino acids are represented by the full name thereof, by the three letter code corresponding thereto, or by the one-letter code corresponding thereto, as indicated in the following table:

| | Three-Letter | One-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, the term “antiviral agent” means a composition of matter which, when delivered to a cell, is capable of preventing replication of a virus in the cell, preventing infection of the cell by a virus, or reversing a physiological effect of infection of the cell by a virus. Antiviral agents are well known and described in the literature. By way of example, AZT (zidovudine, Retrovir® Glaxo Wellcome Inc., Research Triangle Park, N.C.) is an antiviral agent that is thought to prevent replication of HIV in human cells.

As used herein, the term “treatment” or “treating,” is defined as the application or administration of a therapeutic agent, i.e., a compound of the invention (alone or in combination with another pharmaceutical agent), to a subject, or application or administration of a therapeutic agent to an isolated tissue or cell line from a subject (e.g., for diagnosis or ex vivo applications), who has a retroviral infection, a symptom of a retroviral infection or the potential to acquire a retroviral infection, with the purpose to cure, heal, alleviate, relieve, alter, remedy, ameliorate, improve or affect the retroviral infection, the symptoms of the retroviral infection or the potential to acquire the retroviral infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient” or “subject” refers to a human or a non-human animal. Non-human animals include, for example, livestock and pets, such as ovine, bovine, porcine, canine, feline and murine mammals, as well as reptiles, birds and fish. Preferably, the patient or subject is human.

As used herein, the terms “effective amount,” “pharmaceutically effective amount” and “therapeutically effective amount” refer to a nontoxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

“Instructional material,” as that term is used herein, includes a publication, a recording, a diagram, or any other medium of expression that may be used to communicate the usefulness of the compounds of the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

The term “alkyl” refers to saturated aliphatic groups, including straight-chain alkyl groups, branched-chain alkyl groups, cycloalkyl, heterocyclyl, cycloalkyl (alicyclic) groups, alkyl substituted cycloalkyl groups, and cycloalkyl substituted alkyl groups. In preferred embodiments, a straight chain or branched chain alkyl has 8 or fewer carbon atoms in its backbone (e.g., C₁-C₈ for straight chain, C₃-C₈ for branched chain), and more preferably has 6 or fewer carbon atoms in the backbone. Likewise, preferred cycloalkyls have from 3-6 carbon atoms in their ring structure. Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

Examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃H (e.g., —OH), (CR′R″)₀₋₃COR′, (CR′R″)₀₋₃ (substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl or aryl group.

### Compounds of the Invention

The compounds of the invention may be synthesized using techniques well-known in the art of organic synthesis.

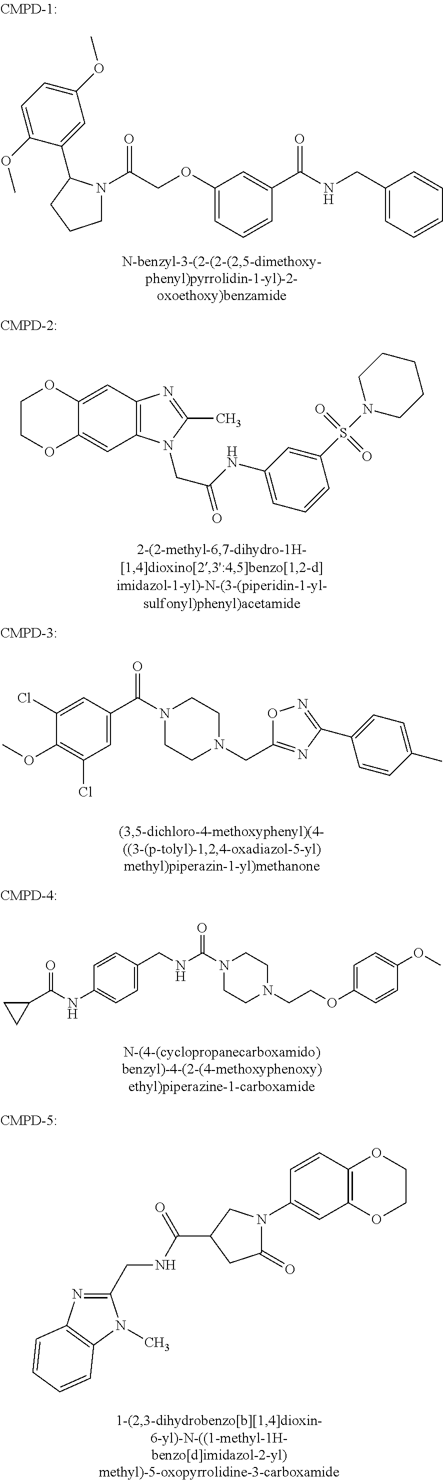

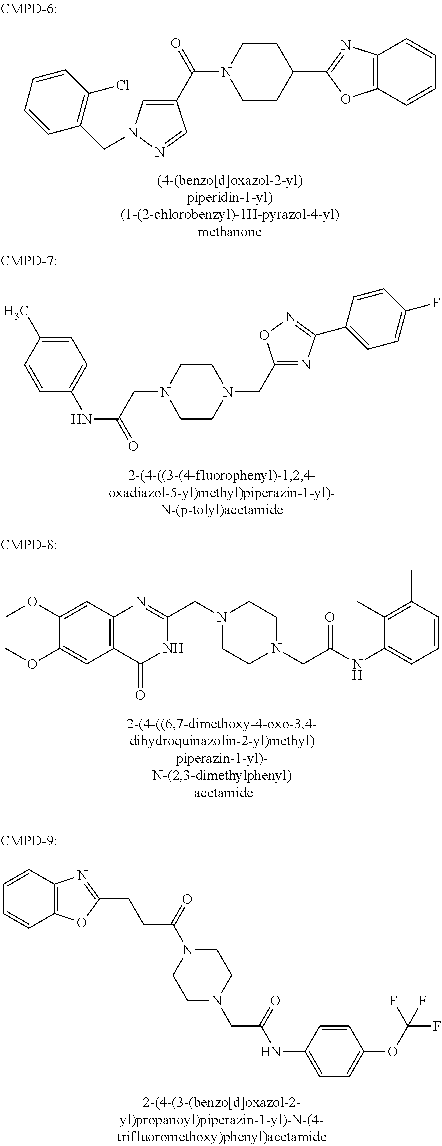 [SMILES: O=C(c1cnn(Cc2ccccc2Cl)c1)N1CCC(c2nc3ccccc3o2)CC1 ; Cc1ccc(NC(=O)CN2CCN(Cc3nc(-c4ccc(F)cc4)no3)CC2)cc1 ; COc1cc2nc(CN3CCN(CC(=O)Nc4cccc(C)c4C)CC3)[nH]c(=O)c2cc1OC ; O=C(CN1CCN(C(=O)CCc2nc3ccccc3o2)CC1)Nc1ccc(OC(F)(F)F)cc1]

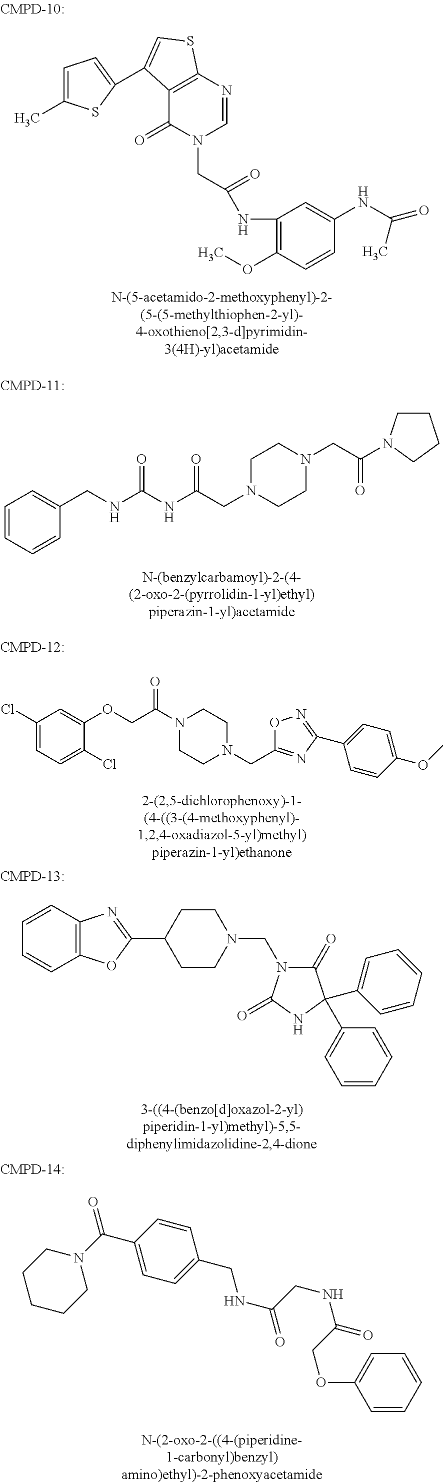 [SMILES: COc1ccc(NC(C)=O)cc1NC(=O)Cn1cnc2scc(-c3ccc(C)s3)c2c1=O ; O=C(CN1CCN(CC(=O)N2CCCC2)CC1)NC(=O)NCc1ccccc1 ; COc1ccc(-c2noc(CN3CCN(C(=O)COc4cc(Cl)ccc4Cl)CC3)n2)cc1 ; O=C1NC(c2ccccc2)(c2ccccc2)C(=O)N1CN1CCC(c2nc3ccccc3o2)CC1 ; O=C(CNC(=O)COc1ccccc1)NCc1ccc(C(=O)N2CCCCC2)cc1]

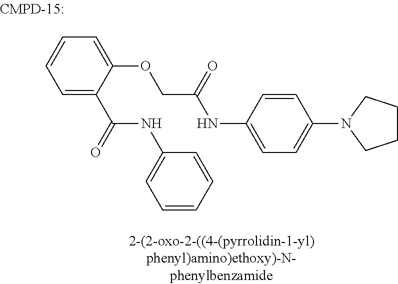 [SMILES: O=C(COc1ccccc1C(=O)Nc1ccccc1)Nc1ccc(N2CCCC2)cc1]

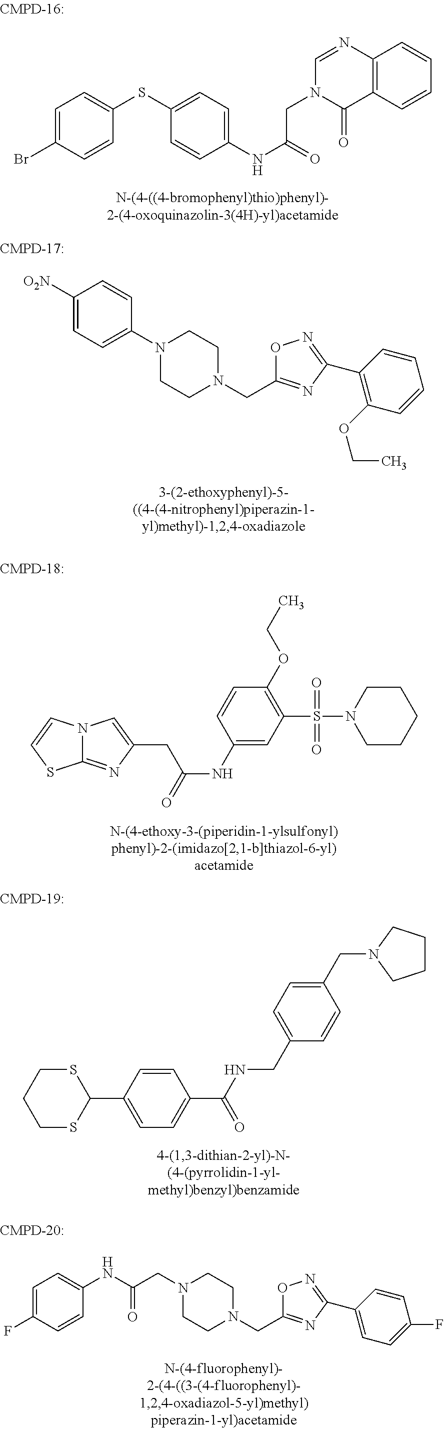 [SMILES: O=C(Cn1cnc2ccccc2c1=O)Nc1ccc(Sc2ccc(Br)cc2)cc1 ; CCOc1ccccc1-c1noc(CN2CCN(c3ccc([N+](=O)[O-])cc3)CC2)n1 ; CCOc1ccc(NC(=O)Cc2cn3ccsc3n2)cc1S(=O)(=O)N1CCCCC1 ; O=C(NCc1ccc(CN2CCCC2)cc1)c1ccc(C2SCCCS2)cc1 ; O=C(CN1CCN(Cc2nc(-c3ccc(F)cc3)no2)CC1)Nc1ccc(F)cc1]

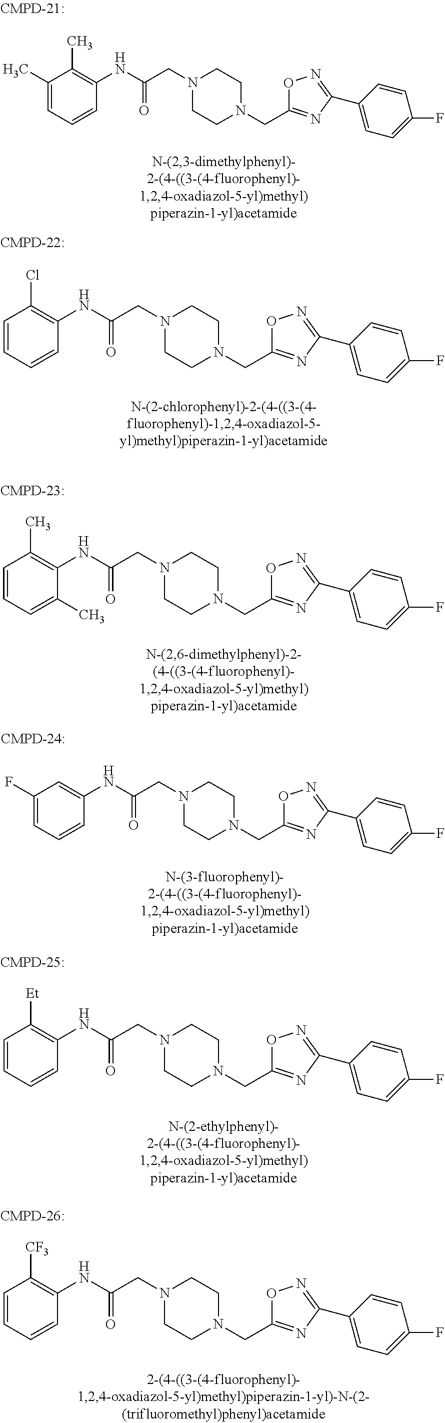 [SMILES: Cc1cccc(NC(=O)CN2CCN(Cc3nc(-c4ccc(F)cc4)no3)CC2)c1C ; O=C(CN1CCN(Cc2nc(-c3ccc(F)cc3)no2)CC1)Nc1ccccc1Cl ; Cc1cccc(C)c1NC(=O)CN1CCN(Cc2nc(-c3ccc(F)cc3)no2)CC1 ; O=C(CN1CCN(Cc2nc(-c3ccc(F)cc3)no2)CC1)Nc1cccc(F)c1 ; CCc1ccccc1NC(=O)CN1CCN(Cc2nc(-c3ccc(F)cc3)no2)CC1 ; O=C(CN1CCN(Cc2nc(-c3ccc(F)cc3)no2)CC1)Nc1ccccc1C(F)(F)F]

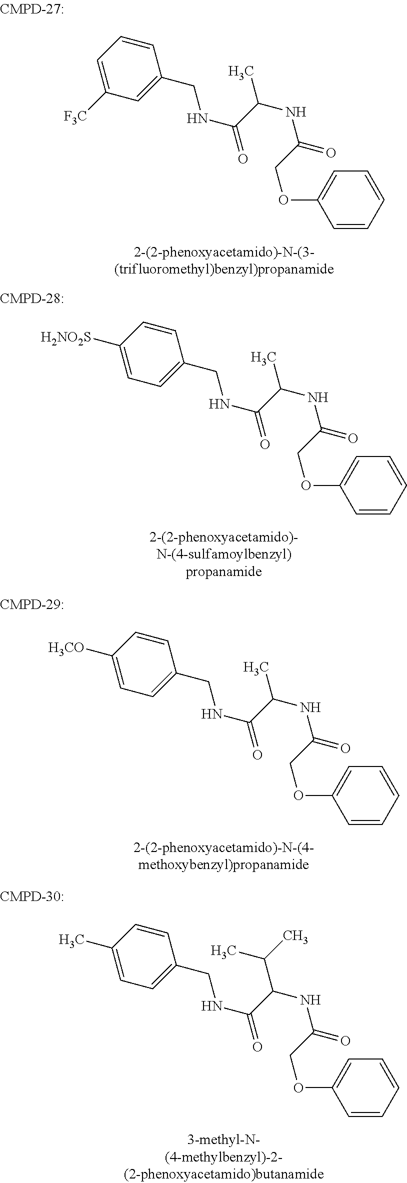 [SMILES: CC(NC(=O)COc1ccccc1)C(=O)NCc1cccc(C(F)(F)F)c1 ; CC(NC(=O)COc1ccccc1)C(=O)NCc1ccc(S(N)(=O)=O)cc1 ; COc1ccc(CNC(=O)C(C)NC(=O)COc2ccccc2)cc1 ; Cc1ccc(CNC(=O)C(NC(=O)COc2ccccc2)C(C)C)cc1]

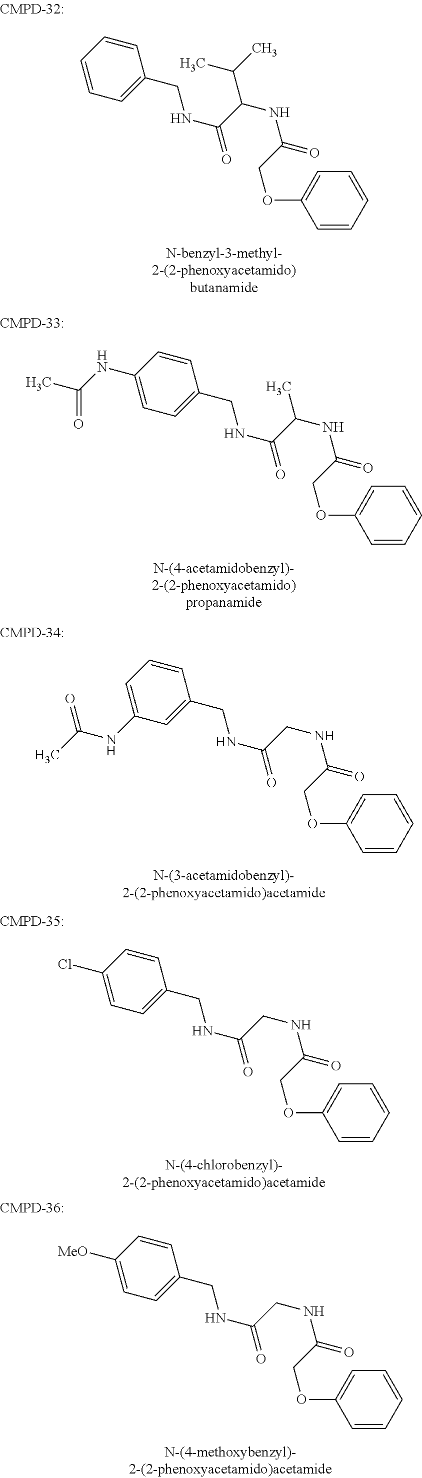 [SMILES: CC(C)C(NC(=O)COc1ccccc1)C(=O)NCc1ccccc1 ; CC(=O)Nc1ccc(CNC(=O)C(C)NC(=O)COc2ccccc2)cc1 ; CC(=O)Nc1cccc(CNC(=O)CNC(=O)COc2ccccc2)c1 ; O=C(CNC(=O)COc1ccccc1)NCc1ccc(Cl)cc1 ; COc1ccc(CNC(=O)CNC(=O)COc2ccccc2)cc1]

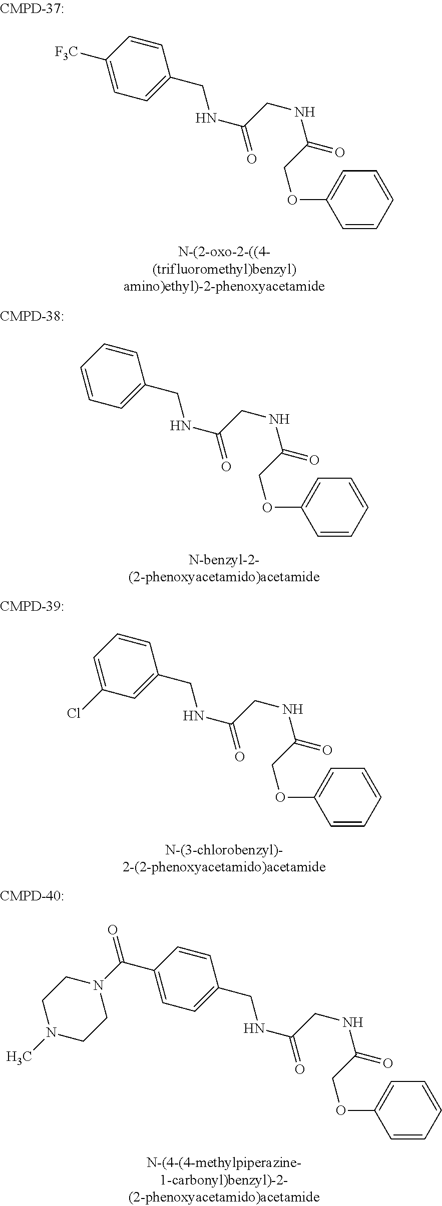 [SMILES: O=C(CNC(=O)COc1ccccc1)NCc1ccc(C(F)(F)F)cc1 ; O=C(CNC(=O)COc1ccccc1)NCc1ccccc1 ; O=C(CNC(=O)COc1ccccc1)NCc1cccc(Cl)c1 ; CN1CCN(C(=O)c2ccc(CNC(=O)CNC(=O)COc3ccccc3)cc2)CC1]

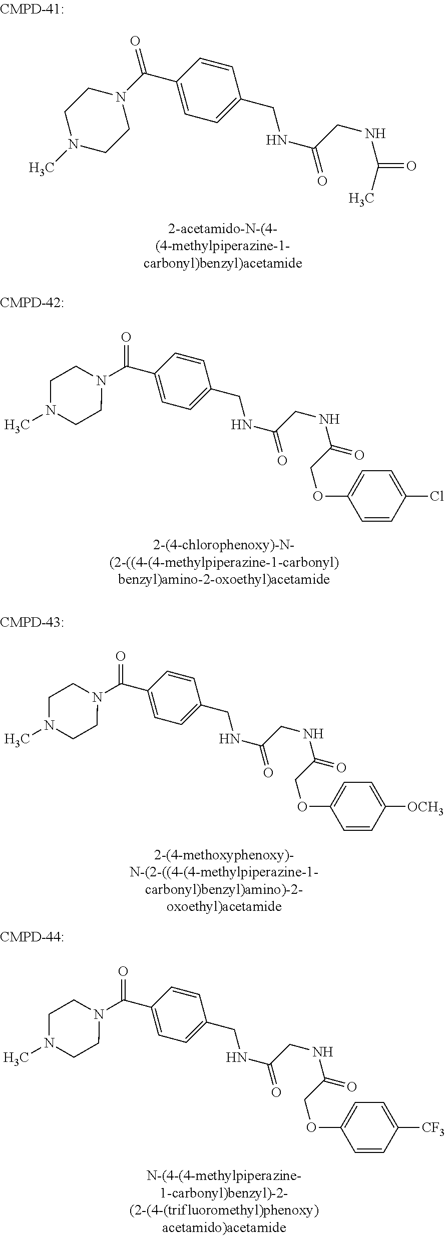 [SMILES: CC(=O)NCC(=O)NCc1ccc(C(=O)N2CCN(C)CC2)cc1 ; CN1CCN(C(=O)c2ccc(CNC(=O)CNC(=O)COc3ccc(Cl)cc3)cc2)CC1 ; COc1ccc(OCC(=O)NCC(=O)NCc2ccc(C(=O)N3CCN(C)CC3)cc2)cc1 ; CN1CCN(C(=O)c2ccc(CNC(=O)CNC(=O)COc3ccc(C(F)(F)F)cc3)cc2)CC1]

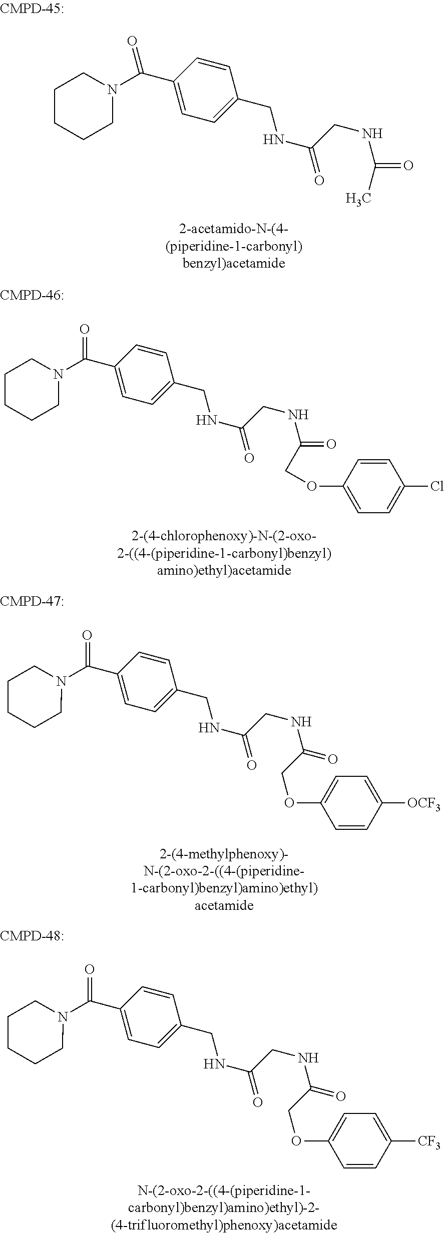 [SMILES: CC(=O)NCC(=O)NCc1ccc(C(=O)N2CCCCC2)cc1 ; O=C(CNC(=O)COc1ccc(Cl)cc1)NCc1ccc(C(=O)N2CCCCC2)cc1 ; O=C(CNC(=O)COc1ccc(OC(F)(F)F)cc1)NCc1ccc(C(=O)N2CCCCC2)cc1 ; O=C(CNC(=O)COc1ccc(C(F)(F)F)cc1)NCc1ccc(C(=O)N2CCCCC2)cc1]

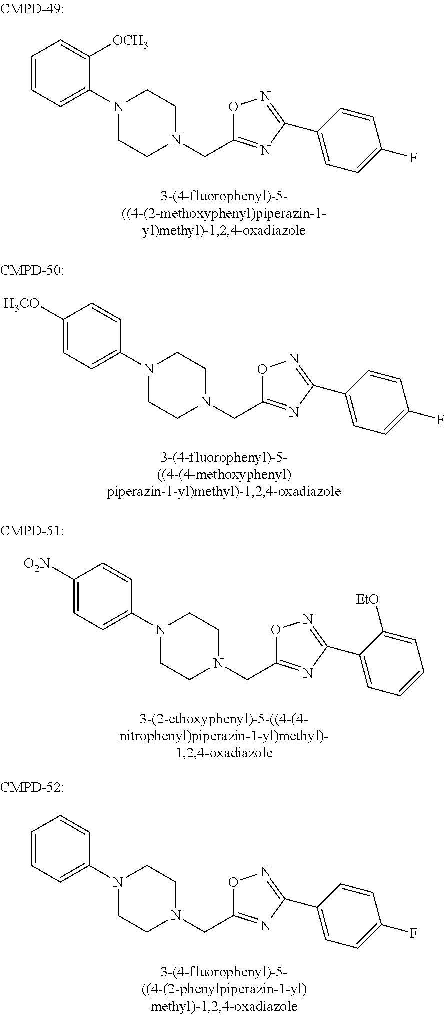 [SMILES: COc1ccccc1N1CCN(Cc2nc(-c3ccc(F)cc3)no2)CC1 ; COc1ccc(N2CCN(Cc3nc(-c4ccc(F)cc4)no3)CC2)cc1 ; CCOc1ccccc1-c1noc(CN2CCN(c3ccc([N+](=O)[O-])cc3)CC2)n1 ; Fc1ccc(-c2noc(CN3CCN(c4ccccc4)CC3)n2)cc1]

In one aspect, the compound of the invention is selected from the group consisting of N-benzyl-3-(2-(2-(2,5-dimethoxyphenyl)pyrrolidin-1-yl)-2-oxoethoxy)benzamide(CMPD-1); 2-(2-methyl-6,7-dihydro-1H-[1,4]dioxino[2′,3′:4,5]benzo[1,2-d]imidazol-1-yl)-N-(3-(piperidin-1-ylsulfonyl)phenyl)acetamide (CMPD-2); (3,5-dichloro-4-methoxyphenyl)(4-((3-(p-toyl)-1,2,4-oxadiazol-5-yl)methyl)piperazin-1-yl)methanone (CMPD-3); N-(4-(cyclopropanecarboxamido)benzyl)-4-(2-(4-methoxyphenoxy)ethyl)piperazine-1-carboxamide (CMPD-4); 1-(2,3-dihydrobenzo[b][1,4]dioxin-6-yl)-N-((1-methyl-1H-benzo[d]imidazol-2-yl)methyl)-5-oxopyrrolidine-3-carboxamide (CMPD-5); 2-(4-(3-(benzo[d]oxazol-2-yl)propanoyl)piperazin-1-yl)-N-(4-(trifluoromethoxy)phenyl)acetamide (CMPD-9); N-(5-acetamido-2-methoxyphenyl)-2-(5-(5-methylthiophen-2-yl)-4-oxothieno[2,3-d]pyrimidin-3(4H)-yl)acetamide (CMPD-10); N-(benzylcarbamoyl)-2-(4-(2-oxo-2-(pyrrolidin-1-yl)ethyl)piperazin-1-yl)acetamide (CMPD-11); 2-(2-oxo-2-((4-(pyrrolidin-1-yl)phenyl)amino)ethoxy)-N-phenylbenzamide (CMPD-15); N-(4-((4-bromophenyl)thio)phenyl)-2-(4-oxoquinazolin-3(4H)-yl)acetamide (CMPD-16); 4-(1,3-dithian-2-yl)-N-(4-(pyrrolidin-1-ylmethyl)benzyl)benzamide (CMPD-19); 2-acetamido-N-(4-(piperidine-1-carbonyl)benzyl)acetamide (CMPD-45); a compound of Formula (I); a compound of Formula (II); a compound of Formula (III); a compound of Formula (IV); a compound of Formula (V); a compound of Formula (VI); a compound of Formula (VII); a compound of Formula (VIII); mixtures thereof and pharmaceutically acceptable carriers thereof, wherein:

the compound of Formula (I) is:

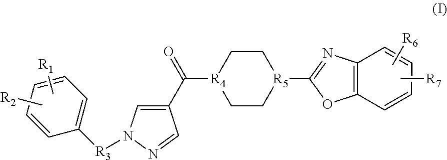

wherein in Formula (I):
   R₁, R₂, R₆ and R₇ are independently H, fluoro, chloro, bromo, C₁-C₆ alkyl, or C₁-C₆ alkoxy,
   R₃ is CH₂ or a chemical bond, and
   R₄ and R₅ are independently CH or N,
   or a pharmaceutically acceptable salt thereof;
      the compound of Formula (II) is:

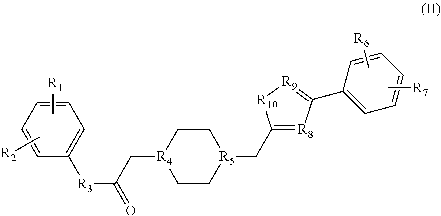

wherein in Formula (II):
   R₁, R₂, R₆ and R₇ are independently H, fluoro, chloro, bromo, —CF₃, C₁-C₆ alkyl, or C₁-C₆ alkoxy,
   R₃ is NH, CH₂ or a chemical bond,
   R₄ and R₅ are independently CH or N,
   R₈ and R₉ are independently CH or N, and
   R₁₀ is O or S;
   or a pharmaceutically acceptable salt thereof;
      the compound of Formula (III) is:

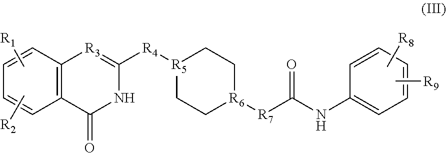

wherein in Formula (III):
   R₁, R₂, R₈ and R₉ are independently H, fluoro, chloro, bromo, C₁-C₆ alkyl, or C₁-C₆ alkoxy,
   R₃ is CH or N,
   R₄ and R₇ are independently CH₂ or a chemical bond, and
   R₅ and R₆ are independently CH or N;
   or a pharmaceutically acceptable salt thereof;
      the compound of Formula (IV) is:

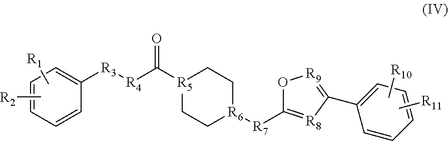

wherein in Formula (IV):
   R₁, R₂, R₁₀ and R₁₁ are independently H, fluoro, chloro, bromo, C₁-C₆ alkyl, or C₁-C₆ alkoxy,
   R₃ is O or NH,
   R₄ and R₇ are independently CH₂ or a chemical bond, and
   R₅, R₆, R₈ and R₉ are independently CH or N;
   or a pharmaceutically acceptable salt thereof;
      the compound of Formula (V) is:

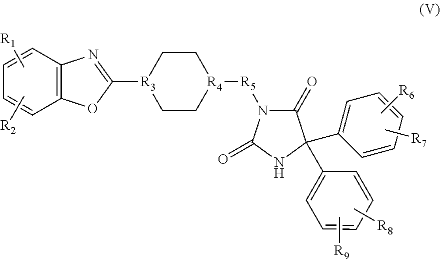

wherein in Formula (V):
   R₁, R₂, R₆, R₇, R₈ and R₉ are independently H, fluoro, chloro, bromo, C₁-C₆ alkyl, or C₁-C₆ alkoxy,
   R₃ are R₄ are independently CH or N, and
   R₅ is CH₂ or a chemical bond;
   or a pharmaceutically acceptable salt thereof;
      the compound of Formula (VI) is:

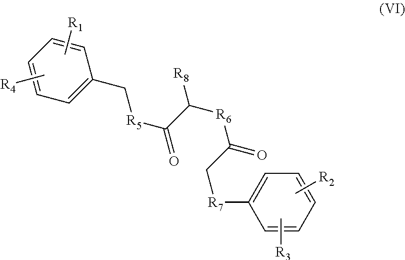

wherein in Formula (VI):
   R₁, R₂, and R₃ are independently H, fluoro, chloro, bromo, C₁-C₆ alkyl, or C₁-C₆ alkoxy,
   R₄ is H, halo, C₁-C₆ alkyl, C₁-C₆ alkoxy, —SO₂NH₂, —NH₂, —NHC(═O)CH₃, —CF₃, or —C(═O)R₉,
   R₅, R₆ and R₇ are independently a chemical bond, O, CH₂ or NH,
   R₈ is H or C₁-C₆ alkyl,
   R₉ is H, C₁-C₆ alkyl, N,N-dimethylamino, N,N-diethylamino, N-morpholinyl, N-pyrrolidinyl, N-piperidinyl, N-piperazinyl, or N′—(C₁-C₆ alkyl)piperazinyl,
   or a pharmaceutically acceptable salt thereof;
      the compound of Formula (VII) is:

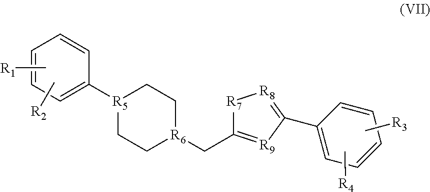

wherein in Formula (VII):
   R₁, R₂, R₃, and R₄ are independently H, nitro, amino, fluoro, chloro, bromo, C₁-C₆ alkyl, or C₁-C₆ alkoxy,
   R₅, R₆, R₈ and R₉ are independently CH or N, and
   R₇ is S or O,
   or a pharmaceutically acceptable salt thereof;
      and the compound of Formula (VIII) is:

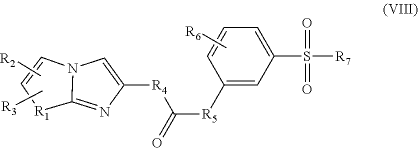

wherein in Formula (VIII):
   R₁ is O, S or —CH═CH—;
   R₂, R₃, and R₆ are independently H, fluoro, chloro, bromo, C₁-C₆ alkyl, or C₁-C₆ alkoxy,
   R₄ and R₅ are independently CH₂ or NH, and
   R₇ is C₁-C₆ alkyl, N,N-dimethylamino, N,N-diethylamino, N-morpholinyl, N-pyrrolidinyl or N-piperidinyl,
   or a pharmaceutically acceptable salt thereof.

In one embodiment, the compound of Formula (I) is:

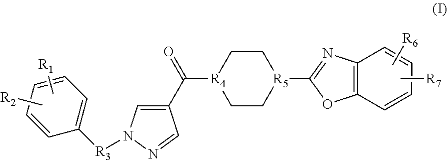

wherein in Formula (I) R₁, R₂, R₆ and R₇ are independently H, fluoro, chloro, bromo, C₁-C₆ alkyl, or C₁-C₆ alkoxy; R₃ is CH₂; R₄ is N; and R₅ is CH, or a pharmaceutically acceptable salt thereof.

In another embodiment, the compound of Formula (II) is:

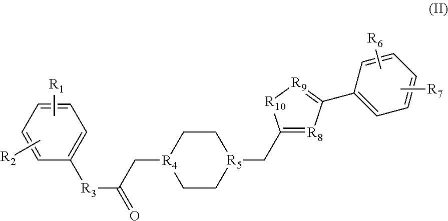

wherein in Formula (II) R₁, R₂, R₈ and R₇₉ are independently H, fluoro, chloro, bromo, —CF₃, C₁-C₆ alkyl, or C₁-C₆ alkoxy; R₃ is NH; R₄ and R₇ are N; R₈ and R₉ are N, and R₁₀ is O, or a pharmaceutically acceptable salt thereof.

In yet another embodiment, the compound of Formula (IV) is:

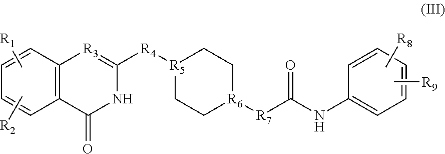

wherein in Formula (III) R₁, R₂, R₈ and R₉ are independently H, fluoro, chloro, bromo, C₁-C₆ alkyl, or C₁-C₆ alkoxy; R₃ is N; R₄ and R₇ are CH₂; and R₅ and R₆ are N; or a pharmaceutically acceptable salt thereof.

In yet another embodiment, the compound of Formula (IV) is:

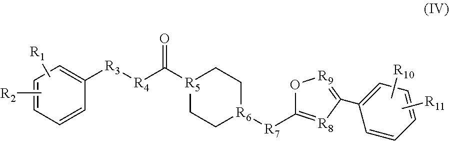

wherein in Formula (IV) R₁, R₂, R₁₀ and R₁₁ are independently H, fluoro, chloro, bromo, C₁-C₆ alkyl, or C₁-C₆ alkoxy; R₃ is O; R₄ and R₇ are CH₂; R₅, R₆, R₈ and R₉ are N; or a pharmaceutically acceptable salt thereof.

In yet another embodiment, the compound of Formula (V) is:

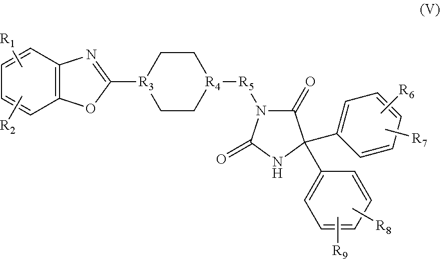

wherein in Formula (V) R₁, R₂, R₆, R₇, R₈ and R₉ are independently H, fluoro, chloro, bromo, C₁-C₆ alkyl, or C₁-C₆ alkoxy; R₃ is CH; R₄ is N; and R₅ is CH₂; or a pharmaceutically acceptable salt thereof.

In yet another embodiment, the compound of Formula (VI) is:

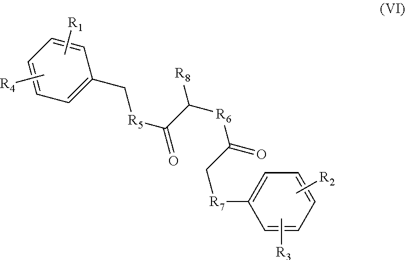

wherein in Formula (VI) R₁, R₂, and R₃ are independently H, fluoro, chloro, bromo, C₁-C₆ alkyl, or C₁-C₆ alkoxy; R₄ is H, halo, C₁-C₆ alkyl, CN—C₆ alkoxy, —SO₂NH₂, —NH₂, —NHC(═O)CH₃, —CF₃, or —C(—O)R₉; R₅ and R₆ are NH; R₇ is O; R₈ is H, methyl or isopropyl; R₉ is H, C₁-C₆ alkyl, N,N-dimethylamino, N,N-diethylamino, N-morpholinyl, N-pyrrolidinyl, N-piperidinyl, N-piperazinyl, or N′—(C₁-C₆ alkyl)piperazinyl; or a pharmaceutically acceptable salt thereof.

In yet another embodiment, the compound of Formula (VII) is:

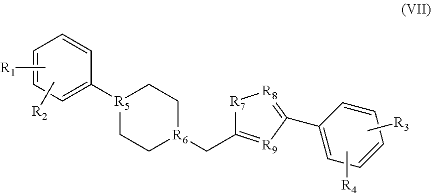

wherein in Formula (VII) R₁, R₂, R₃, and R₄ are independently H, nitro, amino, fluoro, chloro, bromo, C₁-C₆ alkyl, or C₁-C₆ alkoxy, R₅, R₆, R₈ and R₉ are N, and R₇ is O, or a pharmaceutically acceptable salt thereof.

In yet another embodiment, the compound of Formula (VIII) is:

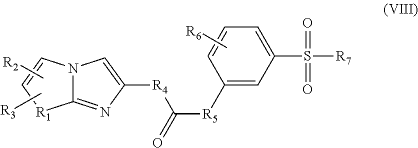

wherein in Formula (VIII) R₁ is S; R₂, R₃, and R₆ are independently H, fluoro, chloro, bromo, C₁-C₆ alkyl, or C₁-C₆ alkoxy; R₄ is CH₂; R₅ is NH; and R₇ is N,N-dimethylamino, N,N-diethylamino, N-morpholinyl, N-pyrrolidinyl or N-piperidinyl; or a pharmaceutically acceptable salt thereof.

In one embodiment, the compound of Formula (I) is (4-(benzo[d]oxazol-2-yl)piperidin-1-yl)(1-(2-chlorobenzyl)-1H-pyrazol-4-yl)methanone (CMPD-6) or a pharmaceutically acceptable salt thereof.

In one embodiment, the compound of Formula (II) is selected from the group consisting of 2-(4-((3-(4-fluorophenyl)-1,2,4-oxadiazol-5-yl)methyl)piperazin-1-yl)-N-(p-tolyl)acetamide (CMPD-7), N-(4-fluorophenyl)-2-(4-((3-(4-fluorophenyl)-1,2,4-oxadiazol-5-yl)methyl)piperazin-1-yl)acetamide (CMPD-20), N-(2,3-dimethylphenyl)-2-(4-((3-(4-fluorophenyl)-1,2,4-oxadiazol-5-yl)methyl)piperazin-1-yl)acetamide (CMPD-21), N-(2-chlorophenyl)-2-(4-((3-(4-fluorophenyl)-1,2,4-oxadiazol-5-yl)methyl)piperazin-1-yl)acetamide (CMPD-22), N-(2,6-dimethylphenyl)-2-(4-((3-(4-fluorophenyl)-1,2,4-oxadiazol-5-yl)methyl)piperazin-1-yl)acetamide (CMPD-23), N-(3-fluorophenyl)-2-(4-((3-(4-fluorophenyl)-1,2,4-oxadiazol-5-yl)methyl)piperazin-1-yl)acetamide (CMPD-24), N-(2-ethylphenyl)-2-(4-((3-(4-fluorophenyl)-1,2,4-oxadiazol-5-yl)methyl)piperazin-1-yl)acetamide (CMPD-25), 2-(4-((3-(4-fluorophenyl)-1,2,4-oxadiazol-5-yl)methyl)piperazin-1-yl)-N-(2-(trifluoromethyl)phenyl)acetamide (CMPD-26), a mixture thereof, and a pharmaceutically acceptable salt thereof.

In another embodiment, the compound of Formula (III) is 2-(4-((6,7-dimethoxy-4-oxo-3,4-dihydroquinazolin-2-yl)methyl)piperazin-1-yl)-N-(2,3-dimethylphenyl)acetamide (CMPD-8) or a pharmaceutically acceptable salt thereof.

In yet another embodiment, the compound of Formula (IV) is 2-(2,5-dichlorophenoxy)-1-(4-((3-(4-methoxyphenyl)-1,2,4-oxadiazol-5-yl)methyl)piperazin-1-yl)ethanone (CMPD-12) or a pharmaceutically acceptable salt thereof.

In yet another embodiment, the compound of Formula (V) is 3-((4-(benzo[d]oxazol-2-yl)piperidin-1-yl)methyl)-5,5-diphenylimidazolidine-2,4-dione (CMPD-13) or a pharmaceutically acceptable salt thereof.

In yet another embodiment, the compound of Formula (VI) is selected from the group consisting of N-(2-oxo-2-((4-(piperidine-1-carbonyl)benzyl)amino)ethyl)-2-phenoxyacetamide (CMPD-14), 2-(2-phenoxyacetamido)-N-(3-(trifluoromethyl)benzyl)propanamide (CMPD-27), 2-(2-phenoxyacetamido)-N-(4-sulfamoylbenzyl)propanamide (CMPD-28), 2-(2-phenoxyacetamido)-N-(4-methoxybenzyl)propanamide (CMPD-29), 3-methyl-N-(4-methylbenzyl)-2-(2-phenoxyacetamido)butanamide (CMPD-30), N-benzyl-3-methyl-2-(2-phenoxyacetamido)butanamide (CMPD-32), N-(4-acetamidobenzyl)-2-(2-phenoxyacetamido)propanamide (CMPD-33), N-(3-acetamidobenzyl)-2-(2-phenoxyacetamido)acetamide (CMPD-34), N-(4-chlorobenzyl)-2-(2-phenoxyacetamido)acetamide (CMPD-35), N-(4-methoxybenzyl)-2-(2-phenoxyacetamido)acetamide (CMPD-36), N-(2-oxo-2-((4-(trifluoromethyl)benzyl)amino)ethyl)-2-phenoxyacetamide (CMPD-37), N-benzyl-2-(2-phenoxyacetamido)acetamide (CMPD-38), N-(3-chlorobenzyl)-2-(2-phenoxyacetamido)acetamide (CMPD-39), N-(4-(4-methylpiperazine-1-carbonyl)benzyl)-2-(2-phenoxyacetamido)acetamide (CMPD-40), 2-acetamido-N-(4-(4-methyl piperazine-1-carbonyl)benzyl)acetamide (CMPD-41), 2-(4-chlorophenoxy)-N-(2-((4-(4-methylpiperazine-1-carbonyl)benzyl)amino)-2-oxoethyl)acetamide (CMPD-42), 2-(4-methoxyphenoxy)-N-(2-((4-(4-methylpiperazine-1-carbonyl)benzyl)amino)-2-oxoethyl)acetamide (CMPD-43), N-(4-(4-methylpiperazine-1-carbonyl)benzyl)-2-(2-(4-(trifluoromethyl)phenoxy)acetamido)acetamide (CMPD-44), 2-(4-chlorophenoxy)-N-(2-oxo-2-((4-(piperidine-1-carbonyl)benzyl)amino)ethyl)acetamide (CMPD-46), 2-(4-methoxyphenoxy)-N-(2-oxo-2-((4-(piperidine-1-carbonyl)benzyl)amino)ethyl)acetamide (CMPD-47), N-(2-oxo-2-((4-(piperidine-1-carbonyl)benzyl)amino)ethyl)-2-(4-(trifluoromethyl)phenoxy)acetamide (CMPD-48), a mixture thereof and a pharmaceutically acceptable salt thereof.

In yet another embodiment, the compound of Formula (VII) is selected from the group consisting of 3-(2-ethoxyphenyl)-5-((4-(4-nitrophenyl)piperazin-1-yl)methyl)-1,2,4-oxadiazole (CMPD-17), 3-(4-fluorophenyl)-5-((4-(2-methoxyphenyl)piperazin-1-yl)methyl)-1,2,4-oxadiazole (CMPD-49), 3-(4-fluorophenyl)-5-((4-(4-methoxyphenyl)piperazin-1-yl)methyl)-1,2,4-oxadiazole (CMPD-50), 3-(2-ethoxyphenyl)-5-((4-(4-nitrophenyl)piperazin-1-yl)methyl)-1,2,4-oxadiazole (CMPD-51), 3-(4-fluorophenyl)-5-((4-phenylpiperazin-1-yl)methyl)-1,2,4-oxadiazole (CMPD-52), a mixture thereof and a pharmaceutically acceptable salt thereof.

In yet another embodiment, the compound of Formula (VIII) is N-(4-ethoxy-3-(piperidin-1-ylsulfonyl)phenyl)-2-(imidazo[2,1-b]thiazol-6-yl)acetamide (CMPD-18) or a pharmaceutically acceptable salt thereof.

In one embodiment, the compound of the invention is selected from the group consisting of N-benzyl-3-(2-(2-(2,5-dimethoxyphenyl)pyrrolidin-1-yl)-2-oxoethoxy)benzamide (CMPD-1); 2-(2-methyl-6,7-dihydro-H-[1,4]dioxino[2′,3′:4,5]benzo[1,2-d]imidazol-1-yl)-N-(3-(piperidin-1-ylsulfonyl)phenyl)acetamide (CMPD-2); (3,5-dichloro-4-methoxyphenyl)(4-((3-(p-tolyl)-1,2,4-oxadiazol-5-yl)methyl)piperazin-1-yl)methanone (CMPD-3); N-(4-(cyclop ropanecarboxam ido)benzyl)-4-(2-(4-methoxyphenoxy)ethyl)piperazine-1-carboxamide (CMPD-4); 1-(2,3-dihydrobenzo[b][1,4]dioxin-6-yl)-N-((1-methyl-1H-benzo[d]imidazol-2-yl)methyl)-5-oxopyrrolidine-3-carboxamide (CMPD-5); (4-(benzo[d]oxazol-2-yl)piperidin-1-yl)(1-(2-chlorobenzyl)-1H-pyrazol-4-yl)methanone (CMPD-6); 2-(4-((3-(4-fluorophenyl)-1,2,4-oxadiazol-5-yl)methyl)piperazin-1-yl)-N-(p-tolyl)acetamide (CMPD-7); 2-(4-((6,7-dimethoxy-4-oxo-3,4-dihydroquinazolin-2-yl)methyl)piperazin-1-yl)-N-(2,3-dimethylphenyl)acetamide (CMPD-8); 2-(4-(3-(benzo[d]oxazol-2-yl)propanoyl)piperazin-1-yl)-N-(4-(trifluoromethoxy)phenyl)acetamide (CMPD-9); N-(5-acetamido-2-methoxyphenyl)-2-(5-(5-methylthiophen-2-yl)-4-oxothieno[2,3-d]pyrimidin-3(4H)-yl)acetamide (CMPD-10); N-(benzylcarbamoyl)-2-(4-(2-oxo-2-(pyrrolidin-1-yl)ethyl)piperazin-1-yl)acetamide (CMPD-11); 2-(2,5-dichlorophenoxy)-1-(4-((3-(4-methoxyphenyl)-1,2,4-oxadiazol-5-yl)methyl)piperazin-1-yl)ethanone (CMPD-12); 3-((4-(benzo[d]oxazol-2-yl)piperidin-1-yl)methyl)-5,5-diphenylimidazolidine-2,4-d lone (CMPD-13); N-(2-oxo-2-((4-(piperidine-1-carbonyl)benzyl)amino)ethyl)-2-phenoxyacetamide (CMPD-14); 2-(2-oxo-2-((4-(pyrrolidin-1-yl)phenyl)amino)ethoxy)-N-phenylbenzamide (CMPD-15); N-(4-((4-bromophenyl)thio)phenyl)-2-(4-oxoquinazolin-3(4H)-yl)acetamide (CMPD-16); 3-(2-ethoxyphenyl)-5-((4-(4-nitrophenyl)piperazin-1-yl)methyl)-1,2,4-oxadiazole (CMPD-17); N-(4-ethoxy-3-(piperidin-1-ylsulfonyl)phenyl)-2-(imidazo[2,1-b]thiazol-6-yl)acetamide (CMPD-18), 4-(1,3-dithian-2-yl)-N-(4-(pyrrolidin-1-ylmethyl)benzyl)benzamide (CMPD-19); N-(4-fluorophenyl)-2-(4-((3-(4-fluorophenyl)-1,2,4-oxadiazol-5-yl)methyl)piperazin-1-yl)acetamide (CMPD-20), N-(2,3-dimethylphenyl)-2-(4-((3-(4-fluorophenyl)-1,2,4-oxadiazol-5-yl)methyl)piperazin-1-yl)acetamide (CMPD-21), N-(2-chlorophenyl)-2-(4-((3-(4-fluorophenyl)-1,2,4-oxadiazol-5-yl)methyl)piperazin-1-yl)acetamide (CMPD-22), N-(2,6-dimethylphenyl)-2-(4-((3-(4-fluorophenyl)-1,2,4-oxadiazol-5-yl)methyl)piperazin-1-yl)acetamide (CMPD-23), N-(3-fluorophenyl)-2-(4-((3-(4-fluorophenyl)-1,2,4-oxadiazol-5-yl)methyl)piperazin-1-yl)acetamide (CMPD-24), N-(2-ethylphenyl)-2-(4-((3-(4-fluorophenyl)-1,2,4-oxadiazol-5-yl)methyl)piperazin-1-yl)acetamide (CMPD-25), 2-(4-((3-(4-fluorophenyl)-1,2,4-oxadiazol-5-yl)methyl)piperazin-1-yl)-N-(2-(trifluoromethyl)phenyl)acetamide (CMPD-26), 2-(2-phenoxyacetamido)-N-(3-(trifluoromethyl)benzyl)propanamide (CMPD-27), 2-(2-phenoxyacetamido)-N-(4-sulfamoylbenzoyl)propanamide (CMPD-28), 2-(2-phenoxyacetamido)-N-(4-methoxybenzyl)propanamide (CMPD-29), 3-methyl-N-(4-methylbenzyl)-2-(2-phenoxyacetamido)butanamide (CMPD-30), N-benzyl-3-methyl-2-(2-phenoxyacetamido)butanamide (CMPD-32), N-(4-acetamidobenzyl)-2-(2-phenoxyacetamido)propanamide (CMPD-33), N-(3-acetamidobenzyl)-2-(2-phenoxyacetamido)acetamide (CMPD-34), N-(4-chlorobenzyl)-2-(2-phenoxyacetamido)acetamide (CMPD-35), N-(4-methoxybenzyl)-2-(2-phenoxyacetamido)acetamide (CMPD-36), N-(2-oxo-2-((4-(trifluoromethyl)benzyl)amino)ethyl)-2-phenoxyacetamide (CMPD-37), N-benzyl-2-(2-phenoxyacetamido)acetamide (CMPD-38), N-(3-chlorobenzyl)-2-(2-phenoxyacetamido)acetamide (CMPD-39), N-(4-(4-methylpiperazine-1-carbonyl)benzyl)-2-(2-phenoxyacetamido)acetamide (CMPD-40), 2-acetamido-N-(4-(4-methylpiperazine-1-carbonyl)benzyl)acetamide (CMPD-41), 2-(4-chlorophenoxy)-N-(2-((4-(4-methylpiperazine-1-carbonyl)benzyl)amino)-2-oxoethyl)acetamide (CMPD-42), 2-(4-methoxyphenoxy)-N-(2-((4-(4-methylpiperazine-1-carbonyl) benzyl)amino)-2-oxoethyl)acetamide (CMPD-43), N-(4-(4-methylpiperazine-1-carbonyl)benzyl)-2-(2-(4-(trifluoromethyl)phenoxy)acetamido)acetamide (CMPD-44), 2-acetamido-N-(4-(piperidine-1-carbonyl)benzyl)acetamide (CMPD-45), 2-(4-chlorophenoxy)-N-(2-oxo-2-((4-(piperidine-1-carbonyl)benzyl)amino)ethyl)acetamide (CMPD-46), 2-(4-methoxyphenoxy)-N-(2-oxo-2-((4-(piperidine-1-carbonyl)benzyl)amino)ethyl)acetamide (CMPD-47), N-(2-oxo-2-((4-(piperidine-1-carbonyl)benzyl)amino)ethyl)-2-(4-(trifluoromethyl)phenoxy)acetamide (CMPD-48), 3-(4-fluorophenyl)-5-((4-(2-methoxyphenyl)piperazin-1-yl)methyl)-1,2,4-oxadiazole (CMPD-49), 3-(4-fluorophenyl)-5-((4-(4-methoxyphenyl)piperazin-1-yl)methyl)-1,2,4-oxadiazole (CMPD-50), 3-(2-ethoxyphenyl)-5-((4-(4-nitrophenyl)piperazin-1-yl)methyl)-1,2,4-oxadiazole (CMPD-51), 3-(4-fluorophenyl)-5-((4-phenylpiperazin-1-yl)methyl)-1,2,4-oxadiazole (CMPD-52), a mixture thereof and a pharmaceutically acceptable carrier thereof.

In another embodiment, the compound of the invention is selected from the group consisting of (4-(benzo[d]oxazol-2-yl)piperidin-1-yl)(1-(2-chlorobenzyl)-1H-pyrazol-4-yl)methanone (CMPD-6); 2-(4-((3-(4-fluorophenyl)-1,2,4-oxadiazol-5-yl)methyl)piperazin-1-yl)-N-(p-tolyl)acetamide (CMPD-7); 2-(4-((6,7-dimethoxy-4-oxo-3,4-dihydroquinazolin-2-yl)methyl)piperazin-1-yl)-N-(2,3-dimethylphenyl)acetamide (CMPD-8); 2-(2,5-dichlorophenoxy)-1-(4-((3-(4-methoxyphenyl)-1,2,4-oxadiazol-5-yl)methyl)piperazin-1-yl)ethanone (CMPD-12); 3-((4-(benzo[d]oxazol-2-yl)piperidin-1-yl)methyl)-5,5-diphenylimidazolidine-2,4-dione (CMPD-13); N-(2-oxo-2-((4-(piperidine-1-carbonyl)benzyl)amino)ethyl)-2-phenoxyacetamide (CMPD-14); 3-(2-ethoxyphenyl)-5-((4-(4-nitrophenyl)piperazin-1-yl)methyl)-1,2,4-oxadiazole (CMPD-17); and N-(4-ethoxy-3-(piperidin-1-ylsulfonyl)phenyl)-2-(imidazo[2,1-b]thiazol-6-yl)acetamide (CMPD-18), or a pharmaceutically acceptable carrier thereof.

In yet another embodiment, the compound of the invention is (4-(benzo[d]oxazol-2-yl)piperidin-1-yl)(1-(2-chlorobenzyl)-1H-pyrazol-4-yl)methanone (CMPD-6), or a pharmaceutically acceptable carrier thereof.

In yet another embodiment, the compound of the invention is 2-(4-((3-(4-fluorophenyl)-1,2,4-oxadiazol-5-yl)methyl)piperazin-1-yl)-N-(p-tolyl)acetamide (CMPD-7) or a pharmaceutically acceptable carrier thereof.

In yet another embodiment, the compound of the invention is 2-(4-((6,7-dimethoxy-4-oxo-3,4-dihydroquinazolin-2-yl)methyl)piperazin-1-yl)-N-(2,3-dimethylphenyl)acetamide (CMPD-8), or a pharmaceutically acceptable carrier thereof.

In yet another embodiment, the compound of the invention is 2-(2,5-dichlorophenoxy)-1-(4-((3-(4-methoxyphenyl)-1,2,4-oxadiazol-5-yl)methyl)piperazin-1-yl)ethanone (CMPD-12), or a pharmaceutically acceptable carrier thereof.

In yet another embodiment, the compound of the invention is 3-((4-(benzo[d]oxazol-2-yl)piperidin-1-yl)methyl)-5,5-diphenylimidazolidine-2,4-dione (CMPD-13), or a pharmaceutically acceptable carrier thereof.

In yet another embodiment, the compound of the invention is N-(2-oxo-2-((4-(piperidine-1-carbonyl)benzyl)amino)ethyl)-2-phenoxyacetamide (CMPD-14) or a pharmaceutically acceptable carrier thereof.

In yet another embodiment, the compound of the invention is 3-(2-ethoxyphenyl)-5-((4-(4-nitrophenyl)piperazin-1-yl)methyl)-1,2,4-oxadiazole (CMPD-17) or a pharmaceutically acceptable carrier thereof.

In yet another embodiment, the compound of the invention is N-(4-ethoxy-3-(piperidin-1-ylsulfonyl)phenyl)-2-(imidazo[2,1-b]thiazol-6-yl)acetamide (CMPD-18), or a pharmaceutically acceptable carrier thereof.

### Common Methods for Inhibitor Discovery

A popular approach for the identification of inhibitors of new protein targets is high-throughput screening, wherein one takes either a large library of chemically diverse compounds available from major vendors or a combinatorially synthesized set of compounds and screens using an appropriate readout using in vitro or ex vivo models. There are two fundamental difficulties with this approach, however. Firstly, the overall number of possible drug-like small molecules typically exceeds by a large margin the number of compounds available within commercially available libraries, therefore reducing the probability of identifying specific inhibitors of a particular protein or process. Secondly, the logistics and costs associated with high-throughput screening using the large number of novel compounds needed to increase the likelihood of inhibitor identification can be prohibitive. To overcome these difficulties a number of virtual screening tools have been developed to reduce the scale of the necessary experiments.

Virtual screening techniques can be broadly classified into two categories: (1) ligand-based methods and (2) structure-based methods, the choice of which is determined by the amount of information known about the protein target and its inhibitors. Ligand-based drug design relies on knowledge of other molecules that bind to the biological target of interest. These other molecules may then be used to derive a pharmacophore that defines the minimum necessary structural characteristics a molecule must possess in order to bind to the target. If no known inhibitor can be provided as a reference, the alternative approach is structure-based drug design (SBDD), which relies on knowledge of the three-dimensional structure of the biological target obtained through methods such as x-ray crystallography or NMR spectroscopy. SBDD approaches can be grouped into two broad categories. The first category involves computationally building ligands within the constraints of a binding pocket within the target protein by assembling either individual atoms or molecular fragments in a stepwise manner. The second category involves discovering potential ligands for a given protein. In this strategy, a large number of potential ligand molecules from virtual databases are screened to find those that fit in the binding pocket of the protein target using computational molecular docking. The former strategy ensures the best fit of a proposed molecule to the protein target binding site, often at the expense of producing unstable or even impossible-to-synthesize compounds. The second approach reduces the chemical space sampled in the study but guarantees that every molecule suggested by the computer exists, can be synthesized, and is readily available.

### Three-Dimensional Models of MA for Docking

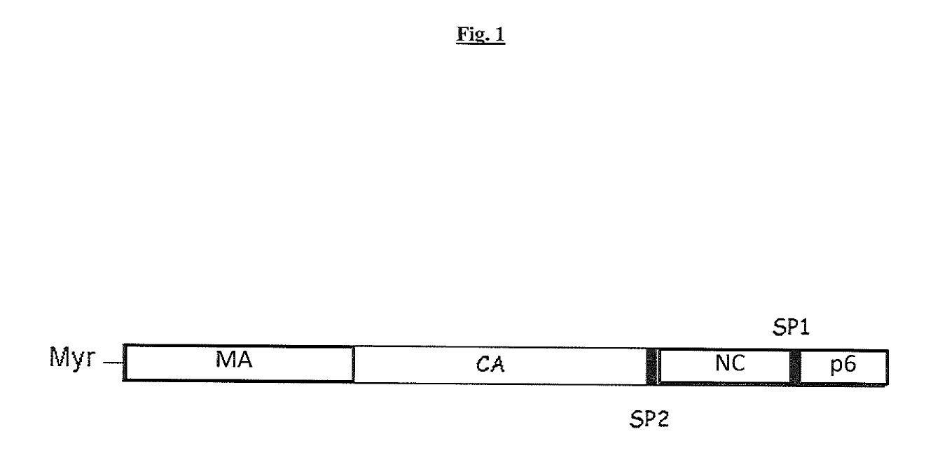

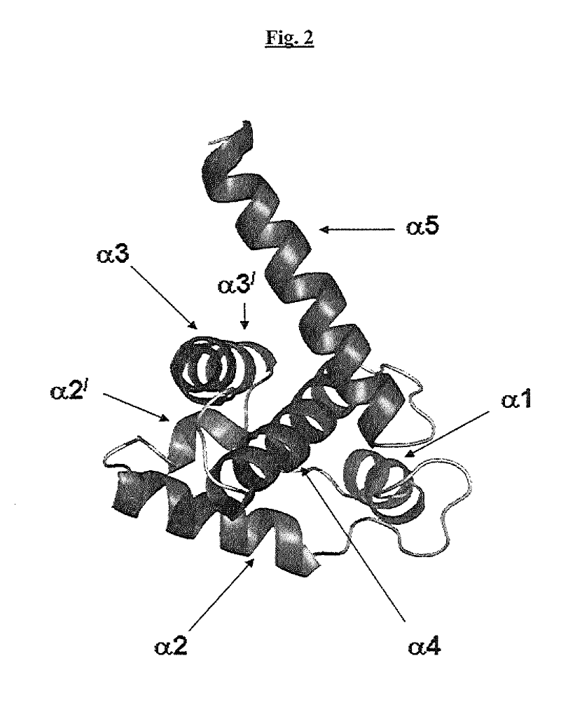

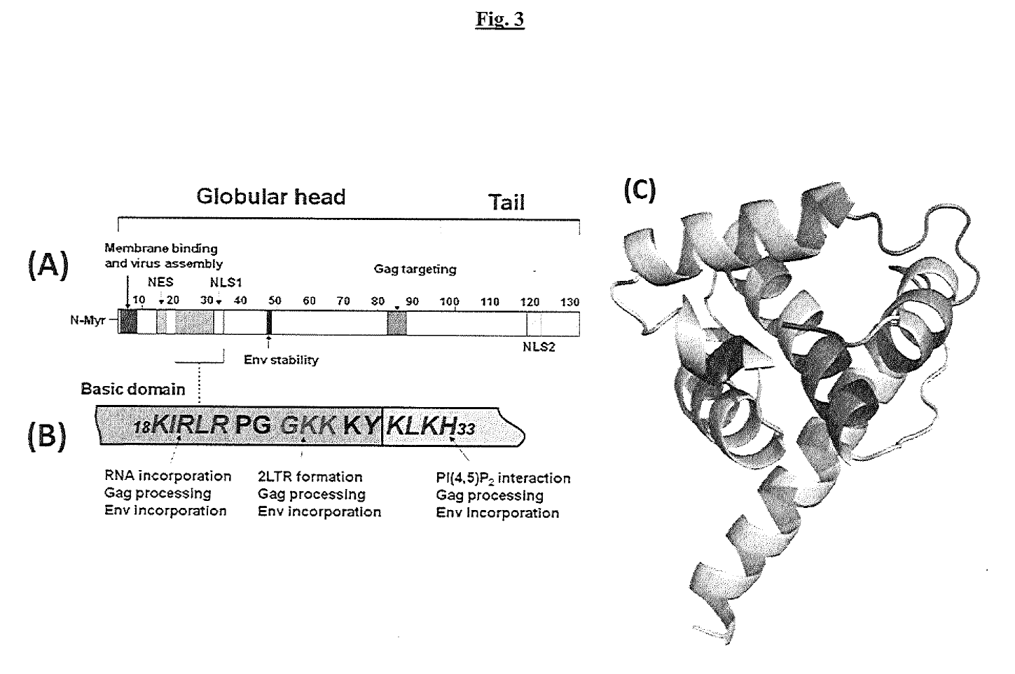

In one aspect of the invention, compounds that bind to HIV-1 MA may be identified using high-throughput computer docking (HTCD). This method uses published structures of MA, with or without a bound ligand, and attempts docking of a library of compounds into a pocket or putative active site of the protein. Published structures may be derived from, in non-limiting examples, NMR or X-ray experiments. HIV-1 MA has been structurally studied by X-ray methods (Kelly et al., 2006, Biochemistry 45(38): 11257-11266) and NMR methods (Saad et al., 2006, Proc. Natl. Acad. Sci. U.S.A. 103(30):11364-11369) (FIGS. 1-3), and such structures may be used to identify inhibitors of MA as described herein.

The MA protein is functionally conserved between all retroviruses. However, it is well established that there is significant genetic diversity both within and between HIV-1 subtypes. Subtypes of HIV-1 differ from one another from 10% to 30% along their entire genomes. Interestingly, these variations are not uniform throughout the entire genome and seem to be more pronounced in the matrix and envelope proteins. An analysis of the HIV-1 sequences in GenBank indicates that relative to the B subtype, the matrix protein exhibits between 22% and 27% sequence diversity at the amino acid level. The most effective targets for the development of small-molecule inhibitors are likely to be functionally critical pockets within the viral proteins that are highly conserved between viral isolates.

The small-molecule library to be docked into the structure may be designed in order to incorporate general structural features known to facilitate binding to the structure of interest, as well as sufficient structural diversity to explore novel binding modes. Furthermore, the small-molecule library may also be designed to include only developable molecules, which combine fair aqueous solubility, good metabolic stability and favorable physical parameters, consistent with the Lipinski analysis (Lipinski et al., 1997, Adv. Drug Del. Rev. 23: 3-25).

In the event that the structure to be used in HTCD experiments presents a bound ligand, the ligand should be removed from the structure. Then, the putative site of binding for the small-molecule library is defined by a three-dimensional box located around the putative binding site.

The interaction of the members of the small-molecule library with the MA protein may be evaluated in terms of the corresponding binding energies, with tight-binding compounds (low K_{D} values) being generally preferred over loose-binding compounds (high K_{D} values). Compounds may be also be evaluated in terms of developability parameters, such as molecular weight, solubility in water and/or DMSO, log P, number of H-bond acceptors, number of H-bond donors, and rotatable bonds, for example. Selection of compounds with tight binding to MA and good developability parameters is thus favored.

In Vitro Antiviral Effects of Small Molecules Identified by HIV-1 Matrix HTCD Experiments

A compound that binds to MA and disrupts at least one of its biological functions may act as an anti-viral agent. A number of available in vitro assays may be used to test disruption of the interactions carried out by HIV-1 MA, but the gold standard is the direct demonstration of antiviral activity. The potential antiviral effects of a molecule identified from the HTCD screen may thus be evaluated by using fully infectious virus and using cells relevant to HIV-1 pathogenesis. The cellular toxicity of the compound, as well as the effects of mutation of the putative compound binding site within MA on their antiviral efficacy, may also be assessed.

### Cell Viability and Cytotoxicity Assays:

Compounds may be screened in PM-1 cells to identify those with undesirable levels of cytotoxicity, which would indicate their unsuitability as drug candidates. Compound cytotoxicity may also inadvertently affect the results of the antiviral activity assays.

In a non-limiting example, compounds may be assayed for cytotoxicity using concentrations (in half-log increments) low enough to be completely non-toxic and, if possible, high enough to result in complete cell death. Exposure times may include, in non-limiting examples, 10 min, 2 h, 24 h, and 5 days. A long exposure time of 5 days may be particularly important because it may reveal levels of cytotoxicity that might affect the virus assay described below.

Briefly, in a non-limiting example, cells are incubated in the absence or presence of each compound at 37° C. under 5% CO₂ and 90% humidity. After exposure and multiple washes, MTT (3-[4,5-dimethylthiazol-2-yl]-2,5-diphenyltetrazolium bromide; 5 mg/mL) is added to each well and incubated for 3 h at 37° C. Following removal of the media by centrifugation for suspension cells, formazan crystals are solubilized for 5 min in 10% Triton ×100 in acidified isopropanol (0.1 N). The resulting solutions are assayed spectrophotometrically at 570 nm (and corrected for non-specific absorption at 690 nm). All assays are performed at least twice with replicate samples for each concentration. Compounds that demonstrate high levels of cytotoxicity in PM1 cells are not considered for further evaluation.

### In Vitro Antiviral Efficacy:

Assays may be conducted using PMI cells (T cells that express CD4 as well as both co-receptors) infected with HIV-1_{LAI}, HIV-1_{BaL}, and HIV-1_{YU-2}. Viral stocks may be produced by transfection of 293T cells with the pLAI.2, pWT/BaL, or pYU-2 infectious molecular clones (IMCs; NIH AIDS Reagent and Reference Program), and half-maximal tissue culture infective dose (TCID₅₀) values may be determined using the method of Reed and Muench (Reed & Muench, 1938, Am. J. Hyg. 27:493-497).

Briefly, in a non-limiting example, PMI cells are infected using a multiplicity of infectivity (MOI) of 0.16 for 2 h. Infected cultures are then washed with RPMI (Roswell Park Memorial Institute) medium and resuspended in 5 mL of RPMI medium at a final cell density of 2×10⁵ cells/mL and added at 1×10⁴ cells per well into 96-well plates containing half-logarithmic dilutions of the test compounds. Five days after infection, virus replication are measured by quantifying HIV-1 p24 antigen present in the supernatants of infected cell cultures using an HIV-1 p24 antigen enzyme-linked immunosorbent assay (ZeptoMetrix Corp., Buffalo, N.Y.). The half-maximal inhibitory concentration (IC₅₀) is calculated as the concentration of compound that causes a 50% decrease in p24 production in the supernatants of infected, compound-treated cells relative to levels of virus produced by infected, compound-free cells. As a means to approach compound specificity, compounds that have activity against HIV-1 are also evaluated for activity against the simian immunodeficiency virus (SIV) and the human T-cell leukemia virus type 1 (HTLV-1).

### Effect of MA Mutation on the Antiviral Effects of Test Compounds

Once antiviral activity and HIV-1 specificity are established for the compounds of interest, dependence of this activity on the presence of HIV-1 MA is assessed. This may be done by altering MA in the context of the provirus, introducing mutations into the infectious molecular clones described above that alter the compound binding site and that are predicted to affect compound affinity.

FIG. 8 illustrates the predicted docked position of compound CMPD-1 and the residues that form its binding site. These include L21, R22, K27, Y29, K30, K32, H33, W36, R76, N80, T81, A83, T97, K98, and L101. Alanine-substitution mutagenesis represents the least biased method to explore ligand-binding sites on proteins of interest, because alanine may usually be accommodated in both solvent-accessible and internal positions on a protein (Levitt, 1978, Biochemistry 17(20):4277-4285; Levitt & Greer, 1977, J. Mol. Biol. 114(2):181-239). Furthermore, alanine may be accommodated readily within several different protein secondary structures, a particularly desirable property in the study of conformationally labile protein regions. Therefore, each of the aforementioned residues are mutated to alanine, with the exception of the single alanine residue (A83), which is replaced by a glycine. Each mutation is first evaluated for its effect on the infectivity of the virus in the absence of compound. This study may use a MA K26T/K27T HIV-1_{NL4-3} mutant virus (which may be obtained from Dr. Akria Ono, Microbiology & Immunology Departments, University of Michigan Medical School, Ann Arbor, Mich.) and viruses singly mutated such that MA residues R22 is mutated to L, K32 is mutated to A, and N80 is mutated to G (which may be obtained from Dr. Eric Freed, HIV Drug Resistance Program, National Cancer Institute, NCI-Frederick, Frederick, Md. 21702-1201). Several of these mutations have been previously characterized as having a detrimental effect on viral replication, especially those in the basic region of MA (Freed et al., 1994, J. Virol, 68(8):5311-5320; Hearps et al., 2008, Biochemistry 47(7):2199-2210; Bhatia, 2007, Virology 369(1):47-54; Bhatia et al., 2008, Virology, in press). Therefore, only viruses with mutations in MA identified as infectious and capable of sustained replication are used to infect PM1 cells using reagents and methods described above. Changes in IC₅₀ concentrations associated with the introduction of MA mutations demonstrate the specificity of the compound for MA and indicate potentially important interactions between the compound and residue that has been mutated.

As an alternative to introducing multiple mutations into three separate infectious molecular clone backbones, MA expression vectors corresponding to each of the three infectious molecular clones are created and serve as the background for the introduction of MA mutations. Levels of mutant MA expression and conformation in 293T cells are assessed by Western blot. These mutated MA expression constructs are then co-transfected with the wild-type infectious molecular clones into 293T cells, and the overexpressed mutant MA molecules are incorporated into the nascent viruses. As described above, mutant viruses are screened for infectivity and sustained replication before proceeding with the antiviral assays.

### Mechanism of Viral Inhibition and MA Binding Properties of Small-Molecule Compounds

HIV-1 MA participates in a number of functions essential to viral replication. A small molecule that is identified from the HTCD screen and displays antiviral activity may interfere with one, some, or all of these stages of viral replication. The results of the infectivity assays described above, using wild-type and mutant viruses, help determine the dependence of the compound's effect on the presence of HIV-1 matrix regions. Further characterization of the compound of interest may be achieved by determining which specific MA-mediated stage in viral replication is disrupted by the compound, and well as by determining the specificity, affinity, kinetics, and stoichiometry of the compound's interaction with HIV-1 MA.

### Infection of Peripheral Blood Mononuclear Cells (PBMCs):

The antiviral activity of promising test compounds identified from the single-round infection assay that display specific, stoichiometric binding to the HIV-1 MA protein is verified using infectious HIV-1 derived from infectious molecular clones (IMCs) and assessing virus replication in PBMCs. Both primary and laboratory-adapted virus may be used. These viruses are representative of subtypes A (KNH1144 and KNH1207, both R5 utilizing; and 96USNG17, X4 utilizing), B (the R5 using NL4-3, YU-2, ADA, BaL isolates, the dual-tropic 89.6 and ELI isolates and X4 utilizing HxBc2 isolate), C (93MW965 and SM145, both R5 utilizing), EA (CM240, R5 utilizing), and D (94UG114.1.6, R5 utilizing). Infectious virus are produced into the culture supernatants of 293T cells transfected with IMCs and used to infect PBMCs isolated from healthy donors, Supernatants containing viral stocks are assessed for the amount of virus by p24gag assay. The drug-resistant isolates available from the NIH AIDS Reagent Program are also included in this analysis; the fusion inhibitor resistant isolate, HIV-1NL4-3 [gp41 (36G) V38A, N42D]; the protease inhibitor resistant isolate, HIV-1NL4-3 [protease, M461/L63P/V82T/184V]; and the reverse transcriptase inhibitor resistant isolate, HIV-1IIIB A17 Variant. Equivalent amounts of virus are incubated with human PBMCs in the presence of increasing amounts of test compound. HIV-1 replication are then followed by periodic measurement of p24gag in culture supernatants. The effects of the compound on the replication of SIV and/or amphotropic MLV are determined in parallel in chimpanzee PBMCs and in murine PBMCs, allowing an assessment of the specificity of any observed effects. Antiviral assays are complemented by experiments in which the effect of compound exposure on PBMC viability are assessed by flow cytometry to detect changes in Annexin V and propidium iodide staining.

### Analysis of HIV-1 Reverse Transcription and Nuclear Import:

The unintegrated viral DNA synthesized during HIV-1 infection includes linear and circular forms. Each of these distinct viral cDNAs may be used as a surrogate marker for events surrounding the completion of reverse transcription and for nuclear import of viral DNA during replication. Therefore, polymerase chain reaction (PCR)-based detection and quantitation of HIV-1 early, late, two-long terminal repeat (2-LTR) circle reverse transcriptase products may be used as a convenient and sensitive means to assess the effect of an identified compound upon nuclear import of viral DNA.

In a non-limiting example, PMI cells are infected with HIV-1_{LA1}, HIV-1_{BaL}, and HIV-1_{YU-2} and cultured in the presence of cell surface CD4 complex monoclonal B4 (to block secondary infection; obtained from the NIH AIDS Reagent and Reference Program) with or without the MA-targeted small-molecule compound. In addition, etravirine (obtained from the NIH AIDS Reagent and Reference Program) is included as a positive control. Total DNA is extracted at 24, 48, and 72 h after infection and HIV-1 early, late, and 2-LTR circle products of reverse transcription are analyzed by fast quantitative PCR (Yoder & Fishel, 2008, J. Virol. Methods 153(2):253-256). Cytoplasmic early and late reverse transcriptase products containing minus-strand strong-stop cDNA or progressing beyond second-strand transfer in reverse transcription, as well as 2-LTR circle products, are detected using primers previously described (Munk et al., 2002, Proc. Natl. Acad. Sci. USA 99(21): 13843-13848; Butler et al., 2001, Nat. Med. 7(5):631-634). This assay reveals the effects of candidate MA inhibitors on the process of reverse transcription and nuclear import.

### Analysis of HIV-1 Proviral DNA Integration:

The quantification of integrated HIV-1 DNA in infected cells may be achieved using the Alu-LTR PCR assay (Brussel & Sonigo, 2003, J. Virol. 77(18):10119-10124; Brussel et al., 2005, Methods Mol. Biol, 304:139-154). Therefore, this PCR-based assay will be used as a convenient and sensitive means to assess the ability of an identified compound to affect the process of proviral DNA integration.

In a non-limiting example, PMI cells are infected with HIV-1_{LA1}, HIV-1_{BaL}, and HIV-1_{YU-2} and cultured in the presence of cell surface CD4 complex monoclonal B4 (to block secondary infection; obtained from the NIH AIDS Reagent and Reference Program) with or without the MA-targeted small-molecule compound. In addition, raltegravir (Isentress/MK-0518; Merck, Whitehouse Station, N.J.) is included as a positive control. Total DNA is extracted at 24, 48, and 72 h after infection and HIV-1 integration is analyzed by the Alu-LTR PCR assay, using primers previously described (Brussel & Sonigo, 2003, J. Virol. 77(18): 10119-10124).

### Analysis of the Efficiency of Viral Release:

The efficiency of virus release may be assessed by calculating the amount of virion-associated Gag as a fraction of total (cell plus virion) Gag synthesized by infected cells. This assessment is achieved using the method of Freed et al. (Freed et al., 1994, J. Virol. 68(8):5311-5320).

Briefly, infected cells are starved of Met/Cys and then metabolically labeled with [³⁵S]Met/Cys for between 2 h to overnight. Virus is purified from the supernatant of infected cells by equilibrium gradient centrifugation. The Gag from viral particles and infected cells is quantified by immunoprecipitation followed by fluorography. If the efficiency of viral release is inhibited by a small-molecule compound, additional assays are performed to determine which process performed by MA is disrupted. Assays include those that look at perturbation of Gag localization, multimerization, and membrane binding (Waheed et al., 2009, Methods Mol, Biol. 485:163-184).

### Envelope Incorporation:

A correlation in the decrease in viral infectivity in the presence of MA-targeted compounds with the efficient incorporation of Env into budding virus may be assessed. PM1 cells are infected with HIV-1_{LA1}, HIV-1_{BaL}, and HIV-1_{YU-2} and cultured in the presence or absence of the MA-targeted small-molecule compound. After a further 24 h of incubation, cell-free virus in the culture supernatants is collected. P4-R5 MAGI cells are then infected by the cell-free supernatant virus using amounts normalized by p24 content. Levels of P4-R5 infection are determined using the Galacto-Star (3-Galactosidase Reporter Gene Assay System for Mammalian Cells (Applied Biosystems, Bedford, Mass.). Cell-free virus produced by the infected PM-1 cells are assessed in parallel by Western blot for virion production (p24, matrix, and gp41) and Env incorporation (gp120). Viruses produced in the presence of effective inhibitors of envelope incorporation are characterized by reduced infectivity and lower levels of gp120 incorporation.

### Direct Binding by SPR:

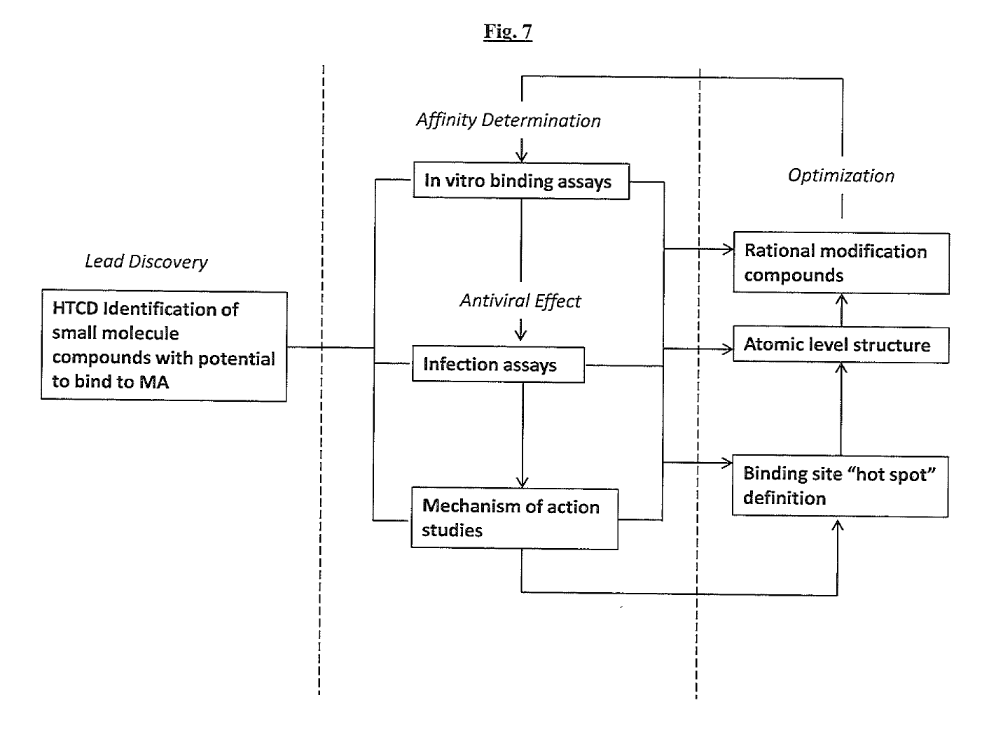

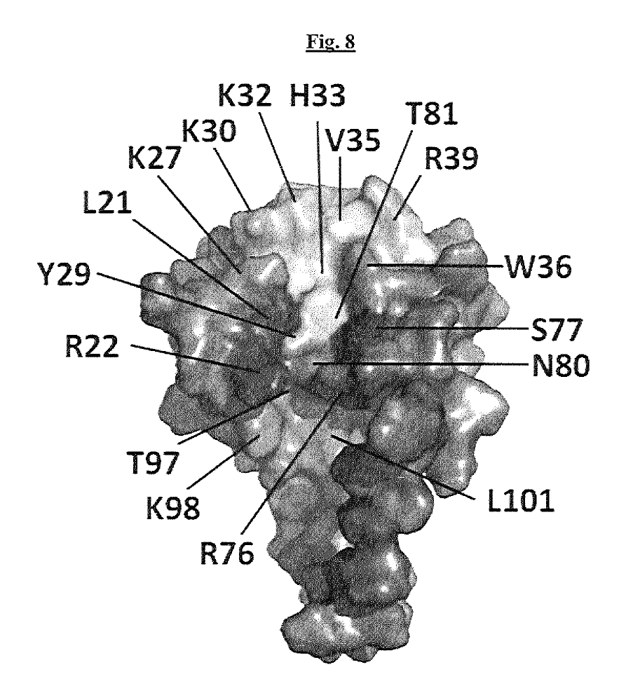

SPR interaction analyses are performed on a Biacore 3000 optical biosensor with simultaneous monitoring of four flow cells or a ProteOn XPR36 SPR array. Immobilization of HIV-1 MA to sensor chips is achieved by standard amide coupling using the recombinant native MA protein illustrated in FIG. 5. A reference surface, containing an irrelevant protein of approximately the same molecular weight such as SUMO is generated using the same conditions and used to correct for background binding and instrument and buffer artifacts.

Direct binding experiments of small molecules to the HIV-1 MA is assessed by injecting increasing concentrations of the compounds over a surface containing the immobilized HIV-1 MA protein to determine affinity, kinetics, and stoichiometry. The density, flow rate, buffer, and regeneration conditions are determined experimentally. The direct binding of the test compounds to SIV_{mac} or HTLV-1 matrix proteins is determined in parallel, allowing a second assessment of their specificity for HIV-1. Once the direct interaction of the compounds with MA has been established, this approach is used to build upon the compound binding site studies described in the subsection “Cell viability and cytotoxicity assays.” This study provides a number of parameters important for the further development of MA-targeted antiviral compounds, including specificity, affinity, stoichiometry, and more importantly association (kₐ) and dissociation (k_{d}) rate constants. FIG. 11 illustrates the interaction of CMPD-18 with immobilized HIV-1 MA and with the protein SUMO.

Assays that examine the effect of a select compound on specific events within the HIV-1 replication cycle (reverse transcription, nuclear import, proviral integration, viral release, envelope incorporation) only indicate the result of compound activity and not the specific mechanism of action associated with the effect. Furthermore, if a compound inhibits multiple functions of MA, the outcome of a particular assay may represent the cumulative effects of multiple mechanisms of action involving MA or other HIV-1 components. These present investigations are designed to identify compounds that are specific inhibitors of HIV-1 MA and that have two attributes that are sought in early drug candidates: high efficacy and little or no cytotoxicity. If compounds with these attributes act through mechanisms that do not involve MA, they may too be considered for future development.

### Specificity Analysis Using SPR:

The SPR direct binding assay may be used to assess the compounds' specificity to HIV-1 MA. The MA domains from the SIVmac, HTLV-1, MuLV, and EIAV gag genes are amplified by polymerase chain reaction (PCR) and cloned into the vector pETHSUL. The retroviral MA proteins is then overproduced and purified using the subtractive methodology outlined above. Retroviral MA proteins are attached to the surface of a sensor chip by amine coupling. A surface to which the SUMO protein has been attached serves as a reference. Direct binding experiments of small molecules to the retroviral MA proteins are assessed by injecting increasing concentrations of the compounds simultaneously over a surface containing the immobilized MA protein and one that contains the SUMO protein.

### Binding Site Interrogation Using SPR:

The putative binding site of the compounds on HIV-1 MA, as identified from the molecular docking experiments, is investigated using two strategies, one that looks at compound competition and the other that uses mutation of binding site residues.

Compound Cross-Competition Analysis of the Compound Binding Site

The compounds identified within this study may bind in a structural groove within the MA globular head that partially overlaps the PI(4,5)P2 binding site. If they do bind in the same region, the compounds should display cross-competition for binding to MA. This cross-competition may be assessed using SPR. Competition analysis is achieved by injecting a saturating concentration of a reference compound (arbitrarily identified from previous experiments), a test compound, and a mixture of the test and reference compounds. Compounds that bind to the same binding pocket as the reference compound shows a signal for the mixture that is nearly identical to the signal observed for the reference compound alone. Compounds that bind at independent sites on MA show a signal for the mixture that corresponds to the sum of the signals determined individually for the reference and test compounds alone. Such an analysis has been implemented in the characterization of compound fragments binding to chymase (Perspicace et al., 2009, J. Biomol, Screen. 14(4):337-349).

Mutational Analysis of the Compound Binding Site:

The compound binding site residues may be altered by site-directed mutagenesis of the HIV-1 MA expression vector. Introducing mutations into the compound binding site may affect compound affinity for the HIV-1 protein, which may then be tested using the SPR assay. FIG. 8 illustrates the predicted collective compound binding site residues for the small molecules identified within this study. Alanine-substitution mutagenesis represents the least biased method to explore ligand-binding sites on proteins of interest, because alanine can usually be accommodated in both solvent-accessible and internal positions on a protein. Furthermore, alanine can be accommodated readily within several different protein secondary structures, a particularly desirable property in the study of conformationally labile protein regions. Therefore, each of the aforementioned residues are mutated to alanine.

### Mutational Analysis of the Compound Binding Site in the Context of the Provirus

Once specific mutations have been identified that alter the compounds' interaction with HIV-1 MA in the direct binding assay, one determines whether those mutations also translate into a loss of efficacy of the compounds in the pseudotype viral assay. Therefore, each of the aforementioned residues are mutated to to alanine. Each mutation is first evaluated for its effect on the infectivity of the virus in the absence of compound. Several of these mutations have been previously characterized as having a detrimental effect on viral replication, especially those in the basic region of MA (Freed et al., 1994, J. Virol. 68(8):5311-5320; Bhatia et al., 2007, Virology 369(1):47-54); Hearps et al., 2008, Biochemistry 47(7):2199-2210; Bhatia et al., 2008, Virology 384(1):233-241). Therefore, only viruses with mutations in MA that allow the production of infectious pseudotypes are used. Changes in half-maximal inhibitory concentrations associated with the introduction of MA mutations are two-fold, demonstrating the specificity of the compound for MA and indicating important functional interactions between the compound and residue that had been mutated.

There exist three potential limitations with the SPR assays designed to monitor the direct binding of small molecules to MA protein. Firstly, the solubility of the compounds in DMSO may be above the tolerance of the instrument. The Biacore 3000 biosensor has a DMSO tolerance of tiup to 8% and the ProteOn XPR36 of 10%. The predicted physical-chemical properties of these compounds suggest that they will be soluble in DMSO concentrations within the functional range of the instrument. If the molecules require more than 8% or 10% organic solvent to be soluble, alternative solubilizations may be employed. Secondly, the sensitivity of the instrument should be sufficient to detect binding of such small molecules to the target protein. Numerous studies indeed document the use of SPR to measure interaction of small-molecule ligand as small as 100 Da with protein targets (Bravman et al., 2006, Anal. Biochem. 358(2):281-288; Cannon et al., 2004, Anal. Biochem. 330(1):98-113; Nordin et al., 2005, Anal. Biochem. 340(2):359-368; Papalia et al., 2006, Anal. Biochem. 359(1):94-105; Stenlund et al., 2006, Anal. Biochem. 353(2):217-225; Wear et al., 2005, Anal. Biochem. 345(2):214-226). The molecules identified from the initial HTCD screen against the structure of HIV-1 MA were all in the range of 390 to 470 Da. As such, no issues are anticipated for the detection of interactions of the identified small molecules with HIV-1 MA. Thirdly, direct attachment method of the HIV-1 MA protein to the sensor surface may result in denaturation of the small-molecule binding site. In a non-limiting approach to overcome this possible issue, a capture strategy may be used: biotinylated HIV-1 MA is attached to the surface of a streptavidin-coated sensor chip. This oriented attachment should circumvent potential problems associated with the random immobilization afforded by the amine coupling strategy.

SPR data analysis is performed using Biaevaluation 4.0 software (Biacore, Piscataway, N.J.) or the ProteOn Manager Software (Bio-Rad, Hercules, Calif.). The responses of a buffer injection and responses from a reference flow cell are subtracted to account for nonspecific binding. To prolong the life of the HIV-1 MA-modified sensor chips, and to increase throughput, the recently described single-cycle kinetics strategy, fitting to a 1:1 kinetic titration model, may be used (Karlsson et al., 2006, Anal. Biochem. 349(1): 136-147). This helps reduce the number of regenerations and allows the quantification of multiple compounds on one surface. Alternatively, “one shot kinetics” is used on the ProteOn XPR36 SPR array (Bravman et al., 2006, Anal. Biochem. 358:281-88). The average kinetic parameters (association [kₐ] and dissociation [k_{d}] rates) generated from a minimum of 4 data sets are used to define equilibrium dissociation constants (K_{D}). The SPR methodology allows the specificity, kinetics, and stoichiometry of interaction to be determined.

The combined results of the studies described above help identify compounds that exert specific effects on HIV-1 replication and verify that HIV-1 MA-dependent processes are being targeted. This information then provides the basis for a detailed investigation into the determinants of activity and affinity, allowing ready prioritization of compounds for further improvement through iterative evaluation of structure-activity relationships and structural investigations (FIG. 7). Such studies comprise: (i) analysis of the binding site for the compounds on the HIV-1 matrix protein p17 by SPR/ITC, coupled with scanning mutagenesis in the compounds' putative binding pocket in matrix protein; (ii) definition of groups critical for affinity and inhibition properties by analysis of the effect of modification of small-molecule R-groups on SPR/ITC binding assays and in vitro viral inhibition assays; (iii) generation of well-diffracting crystals of the low-molecular-weight antiviral compounds complexed with the HIV-1 matrix protein.

### Combination Therapies

The compounds of the present invention are intended to be useful in the methods of present invention in combination with one or more additional compounds useful for treating HIV infections. These additional compounds may comprise compounds of the present invention or compounds, e.g., commercially available compounds, known to treat, prevent, or reduce the symptoms of HIV infections.

In non-limiting examples, the compounds of the invention may be used in combination with one or more of the following anti-HIV drugs:

Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entry and Fusion Inhibitors: maraviroc (Celsentri®, Seizentry®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase hIhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

Routes of administration of any of the compositions of the invention include oral, nasal, rectal, intravaginal, parenteral, buccal, sublingual or topical.

The regimen of administration may affect what constitutes an effective amount. The therapeutic formulations may be administered to the subject either prior to or after the onset of an HIV-1 infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat an HIV-1 infection in the subject. An effective amount of the therapeutic compound necessary to achieve a therapeutic effect may vary according to factors such as the state of the disease or disorder in the subject; the age, sex, and weight of the subject; and the ability of the therapeutic compound to treat an HIV-1 infection in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of an HIV-1 infection in a subject.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin, In one embodiment, the pharmaceutically acceptable carrier is not DMSO alone.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., an HIV-1 antiviral) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of an HIV-1 infection in a subject.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for HIV-1 infection. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing an HIV-1 infection in a subject.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrica, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADR™ White, 32K18400), Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention will depend on the age, sex and weight of the subject, the current medical condition of the subject and the nature of the infection by an HIV-1 being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all starting materials and resins were obtained from commercial suppliers and used without purification. Compounds NBD-556 and ITI-367 were synthesized as below. All other chemicals were purchased from Enamine Ltd. (Kiev, Ukraine).

### NBD-556: N-(4-chlorophenyl), —N′-(2,2,6,6-tetramethylpiperidin-4-yl)-oxalamide

Synthesis of NBD-556 was performed as detailed by Schön et al. (Schon et al., 2006, Biochemistry 45:10973-80).

### ITI-367:

4-(2-methoxyphenyl)-3-[3-(trifluoromethyl)phenyl]-1,2,4-oxadiazol-5-(4H)-one. To a solution of 3-(trifluoromethyl)aniline (2.00 g, 12.4 mmol) and pyridine (6.98 ml, 86.9 mmol) in CH₂Cl₂ (40 ml) was added 2-methoxybenzoyl chloride (2.03 ml, 13.7 mmol) and 4-(dimethylamino)pyridine (3 pieces) and the resulting solution was stirred at room temperature for 3 h. The reaction mixture was then diluted with CH₂Cl₂ (150 ml) and washed with 1 M NaHSO₄ (200 ml), water (100 ml), sat. NaHCO₃ (200 ml), and brine (200 ml). The organic phase was then dried over MgSO₄, filtered and concentrated. Flash chromatography (CH₂Cl₂) provided 2-methoxy-N-[3-(trifluoromethyl)phenyl]benzamide (3.66 g, quant.) as a colorless solid. ¹H NMR (500 MHz, CDCl₃) δ9.93 (br s, 1H), 8.26 (dd, J=7.9, 1.8 Hz, 1H), 8.01 (s, 1H), 7.81 (d, J=8.1 Hz, 1H), 7.49 (ddd, J=8.4, 7.3, 1.9 Hz, 1H), 7.43 (t, J=8.0 Hz, 1H), 7.35 (d, J=7.7 Hz, 1H), 7.12 (ddd, J=7.4, 7.4, 0.8 Hz, 1H), 7.02 (d, J=8.4 Hz, 1H), 4.05 (s, 3H); ¹³C NMR (125 MHz, CDCl₃) δ 163.6, 157.4, 139.1, 133.8, 132.6, 131.4 (q, J_{C-F}=32 Hz), 129.6, 124.6 (q, J_{C-F}=271 Hz), 123.5, 121.9, 121.3, 120.7 (q, J_{C-F}=4 Hz), 117.2 (q, J_{C-F}=4 Hz), 111.7, 56.4; IR (thin film) 3348, 2949, 2844, 1671, 1601, 1556, 1491, 1447, 1336, 1164, 1124, 1020, 755 cm⁻¹; high-resolution mass spectrum (EST) m/z 294.0731 [(M−H)⁺; calculated for C₁₅H₁₁NO₂F₃: 294.0742].

To a flask containing benzamide above (1.44 g, 4.88 mmol) was added benzene (19 ml) followed by PCl₅ (1.12 g, 5.36 mmol). The resulting mixture was heated to reflux for 5 h. The solution was cooled to room temperature and concentrated. The residue was suspended in ether (40 ml) and added to an anhydrous solution of H₂NOH (48.8 mmol, obtained from hydroxylamine hydrochloride (3.39 g, 48.8 mmol) and 1 equiv. of 2% EtONa in EtOH (18.3 ml, 48.8 mmol) in EtOH (40 ml) at 0° C. The reaction mixture was stirred at room temperature for 17 h and concentrated. The residue was partitioned between EtOAc (100 ml) and water (100 ml). The organic layer was washed with saturated NaHCO₃ (100 ml) and brine (100 ml), dried with MgSO₄, and concentrated. Flash chromatography (20%-33% EtOAc/hexane) provided N′-hydroxy-2-methoxy-N-[3-(trifluoromethyl)phenyl)benzimidamide (0.87 g, 57%) as a pale yellow solid. ¹H NMR (500 MHz, CDCl₃) δ 7.48 (d, J=7.4 Hz, 1H), 7.48 (apparent t, J=7.7, 1H), 7.11 (apparent s, 2H), 7.02 (apparent t, J=7.4 Hz, 1H), 6.83 (s, 1H), 6.79-6.72 (m, 2H), 3.38 (s, 3H); ¹³C NMR (125 MHz, CDCl₃) δ157.3, 150.0, 140.6, 131.8, 131.3, 130.8 (q, J_{C-F}=32 Hz), 128.9, 123.9 (q, J_{C-F}=271 Hz), 123.4, 121.0, 119.8, 118.9 (q, J_{C-F}=3 Hz), 116.8 (q, J_{C-F}=4 Hz), 111.2, 55.0; IR (thin film) 3174, 2941, 2839, 1631, 1609, 1498, 1462, 1334, 1124, 928, 756 cm⁻¹; high-resolution mass spectrum (Cl⁺) m/z 311.1007 [(M+H)⁺; calculated for Cl₅H₄N₂O₂F₃: 311.1107].

To a solution of benzimidamide above (0.54 g, 1.74 mmol) in THF (40 ml) was added 1,1′-carbonyldimidazole (339 mg, 2.09 mmol) and the resulting solution stirred for 4 h at 50° C. After concentration, the residue was dissolved in EtOAc (150 ml) and washed with saturated NaHCO₃ (100 ml) and brine (100 ml), dried over MgSO₄, and concentrated. Flash chromatography (11% EtOAc/hexane) provided 4-(2-methoxyphenyl)-3-[3-(trifluoromethyl)phenyl]-1,2,4-oxadiazol-5-(4H)-one (ITI-367) (454 mg, 78% yield) as a colorless solid. ¹H NMR (500 MHz, CDCl₃) δ 7.58 (dd, J=7.5, 1.7, 1H), 7.51 (ddd, J=8.0, 8.0, 1.6 Hz, 1H), 7.46 (apparent t, J=8.1 Hz, 1H), 7.45 (s, 1H), 7.33 (d, J=8.0 Hz, 1H), 7.10 (apparent t, J=7.5 Hz, 1H), 6.77 (d, J=8.4 Hz, 1H); 3.31 (s, 3H); ¹³C NMR (125 MHz, CDCl₃) δ157.7, 157.2, 156.8, 134.5, 133.6, 131.8 (q, J_{C-F}=33 Hz), 131.3, 129.9, 128.4, 125.5 (q, J_{C-F}=4 Hz), 123.4 (q, J_{C-F}=273 Hz), 122.4 (q, J_{C-F}=4 Hz), 121.6, 112.1, 111.5, 55.0; IR (thin film) 1779, 1597, 1470, 1343, 1167, 1129, 757 cm⁻¹; high-resolution mass spectrum (ESI⁺) m/z 337.0801 [(M+H)⁺; calculated for C₁₆H₁₂N₂O₃F₃: 337.0800].

### Virtual Compound Libray:

The structures of ligands for virtual screening were taken from the Enamine Screening Collection library. The Enamine library was clustered using the Jarvis-Patrick algorithm (Jarvis & Patrick, 197, IEEE Transactions on Computers 22:1025-34; Li, 2006, J. Chem. Inf. Model. 46:1919-23; Willett, 1987, Similarity and Clustering in Chemical Information Systems. Research Studies Press Letchworth, Hertfordshire, England) implemented in QUANTUM. The measure of dissimilarity (“distance”) between the molecules was determined by Tanimoto similarity calculated with Daylight fingerprints of the molecules (Daylight Chemical Information Systems, Inc.; Aliso Viejo, Calif.). The clustering parameters were selected such that a reasonable number of clusters (˜72 k) was obtained. The compounds representing the cluster centroids were taken for subsequent screening.

As a result the library of 1M compounds was reduced to a library of roughly 72 k cluster representatives of appropriate molecular weight. The compounds were prepared for docking by extraction from the Enamine-supplied sdf files and processing through the QUANTUM structure recovery and typization software components in batch mode.

### Cells:

Human embryonic kidney 293T cells were cultured in Dulbecco modified Eagle medium supplemented with 10% heat-inactivated FBS, L-glutamine, and antibiotics. Human astroglioma U87 cells stably transfected for the expression of CD4 and CXCR4 (obtained from HongKui Deng and Dan Littman, through the AIDS Research and Reference Reagent Program, Division of AIDS, NIAID, NIH) (Bjorndal, A., et al., 1997, J Virol 71:7478-7487) were cultured in Dulbecco modified Eagle medium supplemented as above plus puromycin and G418 (Geneticin, Gibco BRL Life Technologies, Grand Island, N.Y.).

### Production of Pseudotype:

Single-round infectious envelope-pseudotyped luciferase-reporter viruses were produced in 293T cells co-transfected by calcium phosphate precipitation (Profection Mammalian Transfection System; Promega, Madison, Wis.) with the envelope-deficient HIV-1 NL4-3 vector (pNL4-3-LucR⁺E⁻; a gift of N. R. Landau, New York University) (Connor et al., 1995, Virology 206:935-944), which carries the luciferase-reporter gene; and the HxBc2 envelope-expressing vector (obtained from Kathleen Page and Dan Littman through the AIDS Research and Reference Reagent Program, Division of AIDS, NIAID, NIH) (Page et al., 1990, J Virol 64:5270-5276). All MA mutations within the pNL4-3-LucR⁺E⁻ vector were synthesized and validated by Genescript (Piscataway, N.J.). After 6 h of incubation at 37° C., the DNA-containing medium was removed, cells were washed, and fresh medium with or without MA-targeted inhibitory (MTI) compounds (at concentrations between 3.1 and 100 μM) was added. Supernatants containing the envelope-pseudotyped viruses were collected 2 days later, clarified by centrifugation, aliquoted, and stored at −80° C. until use. Pseudotype stocks were quantified by p24^{gag} content (Advanced Bioscience Laboratories, Kensington, Md.).

### Single-Round Infection Assays:

To determine whether the compounds of the invention affect early stages of the viral life cycle, 50 μL medium alone or containing MTI compounds at twice the desired final concentration (between 3.1 and 100 μM) was added to the U87.CD4.CXCR4 target cells plated in 96-well plates, and 50 μL of pseudotype stocks (not treated during production in 293T cells) was added immediately. Alternatively, to study the inhibitory effects of the compounds of the invention during the late stages of the viral life cycle, untreated or MTI-treated pseudotype stocks were diluted 1:10 with fresh medium and 100 μL was used for infection of U87.CD4.CXCR4 target cells; this dilution ensured that any effect observed was due to the concentration of the compound present during viral production and not to the residual amount of compound present during infection of target cells. Assessment of the effects of the compounds of the invention on early and late stages of infection with pseudotype stocks produced with a luciferase-reporter vector encoding a mutant MA was done in the same manner. Infections were performed for 2 days and quantified by measuring luciferase activity in cell lysates (Luciferase Assay System, Promega) using a microplate luminometer (GloMax, Promega).

### Compound Cytotoxicity:

Supernatants from target cells infected with or without compounds to test their effects at early stages, and pseudotype stocks produced with or without compounds to evaluate their effects at late stages, were used for analysis of cytotoxicity. These effects were estimated through the presence in culture supernatants of LDH (a stable cytoplasmic enzyme), which would indicate plasma membrane damage, using a colorimetric assay (LDH Cytotoxicity Detection kit; Clontech, Mountain View, Calif.).

### Cloning, Overexpression, and Purification of HIV-1 LAI Matrix:

Cloning and overexpression of the HIV-1 MA protein was performed using the protocol outlined below. The matrix region (SEQ ID NO: 1) of the HIV-1 gag gene was amplified from plasmid pLAT using primers designed to facilitate ligation-independent cloning into the vector pETHSUL (Weeks et al., 2007, Protein Expr. Purif, 53(1):40-50). This vector was designed for the insertion of genes of interest in frame with an N-terminal SUMO (small ubiquitin-related modifier) tag (SEQ ID NO:2; Weeks et al., 2007, Protein Expr. Purif. 53(1):40-50).

The recombinant pETHSUL plasmid was verified for the presence of matrix region insert by restriction digestion and sequence analysis (Genewiz, Inc., South Plainfield, N.J.). The resultant vector was designated pSUMO-MA.

The purification of H₆SUMO-MA was achieved via immobilized metal affinity chromatography using TALONspin cobalt resin affinity columns (ClonTech Laboratories, Inc., Mountain View, Calif.).

The Escherichia coli strain BL21 (DE3) Codon+-RIL (Stratagene, La Jolla, Calif.) was used for expression of H₆SUMO-MA from pSUMO-MA. Two milliliters of Luria-Bertani broth, containing 100 μg/mL⁻¹ ampicillin and 50 μg/mL⁻¹ chloramphenicol, were inoculated with a single transformed colony and allowed to grow at 37° C. for 9 h. A total of 100 μL of the pre-culture was used to inoculate 100 mL of the autoinducing media ZYP-5052 (Studier, 2005, Protein Expr Purif 41:207-34) containing 100 μg/mL⁻¹ ampicillin and 34 μg/mL⁻¹ chloramphenicol. The culture was grown at 30° C. for 16 h. Cells were harvested by centrifugation at 1076×g for 20 min at 4° C. and the pellet was suspended in 30 mL PBS (Roche, Nutley, N.J.) containing 2.5 mM imidazole. Cells were lysed by sonication and the supernatant clarified by centrifugation at 11,952×g (SS-34, Sorvall RC 5C Plus) for 20 min at 4° C. The supernatant was removed and applied to a TALON cobalt resin affinity column (ClonTech), previously equilibrated with PBS (Roche). Loosely bound proteins were removed via seven-column volumes of PBS containing 7.5 mM imidazole. Tightly associated proteins were eluted in three-column volumes of PBS containing 250 mM imidazole.

The eluates were then pooled and made 1 mM with respect to ethylenediaminetetraacetic acid (EDTA). To this pooled sample, 10 μg of a recombinant His₆-tagged form of the catalytic domain (dtUD1) of the Saccharomyces cerevisiae SUMO hydrolase was added (Weeks et al., 2007, Protein Expr Purif 53:40-50). Cleavage was allowed to proceed for 4 h at 18° C. Following cleavage, the sample was dialyzed at 4° C. overnight against 2 L of PBS to remove any imidazole. After dialysis, the dtUD1-catalyzed cleavage reaction was subjected to a second cobalt affinity purification using the TALON cobalt resin affinity column. In this purification step, however, the cleaved MA protein passes straight through the column owing to removal of the hexa-histidine tag. Subsequently, the subtractively purified MA was dialyzed against 25 mM Tris-HCl, pH 8.0, 10% glycerol, at 4° C. overnight. This dialyzed sample was then filtered and loaded onto a 5 mL Hi-Trap Q HP column (GE Healthcare, Chalfont, UK). The flow-through, containing the MA protein, was concentrated, flash frozen in liquid nitrogen, and stored at −80° C. Samples at each stage were taken and analyzed via sodium dodecyl sulfatepolyacrylamide gel electrophoresis (FIG. 5).

Following the subtractive purification, the recombinant MA was dialyzed into 25 mM Tris-HCl, pH 8.0, 10% glycerol. After dialysis, the MA was filtered and loaded onto a 5-mL Hi-Trap Q HP (GE Healthcare, Chalfont St. Giles, UK). The flowthrough was subsequently collected, concentrated, flash frozen in liquid nitrogen, and stored at −80° C. for future use. Using this procedure, the purified HIV-1 matrix protein was produced from Escherichia coli at a yield of 15 mg·L⁻¹ and at a purity of greater than 95%.

Interaction of Compounds of the Invention with HIV-1 MA as Monitored by Structural Protein as Monitored by Surface Plasmon Resonance (SPR):

An assay was devised to monitor the interactions of small-molecule compounds to HIV-1 MA using SPR. In this assay, HIV-1_{LA1} MA was immobilized to the flow cells of a CM5 sensor chip to high density (˜1.3 kRU) or a CM7 sensor chip to high density (19 kRU) using standard amine coupling. A reference surface was similarly created by immobilizing the SUMO protein or a nonspecific antibody ARC4033 (antimouse/rat interferon-γ: BioSource). In small-molecule assays, the buffering system used is important due to the low responses obtained in binding assays. Among the most commonly used buffers, phosphate-buffered saline (PBS) was found to be the most suitable for the MA small-molecule binding assays, as with a Tris-based buffer, variable and drifting baselines were observed, complicating evaluation of the data. A stock solution of the MTI compounds was prepared by dissolving in 100% dimethyl sulfoxide (DMSO) to a final concentration of 10 mM.

To prepare the sample for analysis, 30 μL of the stock solution of the compound of the invention was added to sample preparation buffer (PBS, pH 7.2) to a final volume of 1 mL and mixed thoroughly. Preparation of analyte in this manner ensured that the concentration of DMSO was matched with that of running buffer with 3% DMSO. Lower concentrations of each compound were then prepared by two-fold serial dilutions into running buffer (PBS, 3% DMSO, pH 7.2). These compound dilutions were then injected over the control and MA surfaces at a flow rate of 30 μL min⁻, for a 4-min association phase, followed by a 5-min dissociation phase.

In one aspect, owing to the relatively fast dissociation rates of these compounds, regeneration of these surfaces was accomplished by extensive washing of the system followed by a 5 min wait phase, a second wash, and a final 15 min wait phase. To secure complete regeneration, a buffer injection (5 min^{on}/5 min^{off}) was performed before each subsequent injection of compound.

Response to a small-molecule compound (NBD-556) specific for HIV-1 gp120 was also monitored to ensure specificity. A comparison of the interaction of CMPD-18 with HIV-1 MA versus SUMO is shown in FIG. 11. These data show specific interaction of compound CMPD-18 with HIV-1 MA and demonstrate the ability to monitor small molecule-MA interactions using SPR.

### Smuface Plasmon Resonance Data Analysis:

Data analysis was performed using BIAEvaluation 4.0 software (GE Healthcare). The responses of a buffer injection and responses from the ARC4033-immobilized reference cell were subtracted to account for nonspecific binding. The average maximum response was generated from a minimum of 3 data sets and was used to define the standard deviation for each compound binding to the HIV-1 MA protein.

### Anti-HIV Efficacy Evaluation in Human Peripheral Blood Mononuclear Cells:

HIV human peripheral blood mononuclear cell (PBMC) assays were performed as described previously (Lanier et al., 2010, Antimicrob Agents Chemother 54:2901-2909 and Ptak et al., 2008, Antimicrob Agents Chemother 52:1302-1317). Briefly, fresh PBMCs, seronegative for HIV and hepatitis B virus (HBV), were isolated from blood samples of the screened donors (Biological Specialty Corporation, Colmar, Pa.) by using lymphocyte separation medium (LSM; density, 1.078±0.002 g/ml; Cellgro; Mediatech, Inc.) by following the manufacturer's instructions. Cells were stimulated by incubation in 4 μg/mL phytohemagglutinin (PHA; Sigma) for 48 to 72 h. Mitogenic stimulation was maintained by the addition of 20 U/mL recombinant human interleukin-2 (rhIL-2; R&D Systems, Inc.) to the culture medium. PHA-stimulated PBMCs from at least two donors were pooled, diluted in fresh medium, and added to 96-well plates at 5×10⁴ cells/well. Cells were infected (final multiplicity of infection [MOI] of ≅0.1) in the presence of nine different concentrations of test compounds (triplicate wells/concentration) and incubated for 7 days. To determine the level of virus inhibition, cell-free supernatant samples were collected for analysis of reverse transcriptase activity (Buckheit et al., 1991, AIDS Res Hum Retroviruses 7:295-302). Following removal of supernatant samples, compound cytotoxicity was measured by the addition of 3-(4,5-dimethylthiazol-2-yl)-5-(3-carboxymethoxy-phenyl)-2-(4-sulfophenyl)-2H-tetrazolium (MTS; CellTiter 96 reagent; Promega) by following the manufacturer's instructions.

Virus isolates were obtained from the NIH AIDS Research and Reference Reagent Program, Division of AIDS, NIAID, NIH, as follows: HIV-1 Group M isolates 92UG031 (Subtype A, CCR5-tropic), 92BR030 (Subtype B, CCR5-tropic), 92BR025 (Subtype C, CCR5-tropic), 92UG024 (Subtype D, CXCR4-tropic), and 93BR020 (Subtype F, CCR5/CXCR4 Dual-tropic) from the UNAIDS Network for HIV Isolation and Characterization (Gao et al., 1994, AIDS Res Hum Retroviruses 10:1359-1368); HIV-1 Group M isolate 89BZ167 (Subtype B, CXCR4-tropic; also referred to as “89BZ_{—}167”, “89_BZ167”, “BZ167” or “GS 010”) from Dr. Nelson Michael (Brown et al., 2005, J Virol 79:6089-6101; Jagodzinski et al., 2000, J Clin Microbiol 38:1247-1249; Michael et al., 1999, J Clin Microbiol 37:2557-2563 and Vahey et al., 1999, J Clin Microbiol 37:2533-2537); HIV-1 Group M isolate 93IN101 (Subtype C, CCR5-tropic) from Dr. Robert Bollinger and the UNAIDS Network for HIV Isolation and Characterization (Gao et al., 1994, AIDS Res Hum Retroviruses 10:1359-1368); HIV-1 Group M isolate CMU08 (Subtype E, CXCR4-tropic) from Dr. Kenrad Nelson and the UNAIDS Network for HIV Isolation and Characterization (Gao et al., 1994, AIDS Res Hum Retroviruses 10:1359-1368); HIV-1 Group M isolate G3 (Subtype G, CCR5-tropic) from Alash'le Abimiku (Abimiku 1994, Aids Research and Human Retroviruses 10:1581-1583); HIV-1 Group O isolate BCF02 (CCR5-tropic) from Sentob Saragosti, Frangoise Brun-Vézinet, and François Simon (Loussertajaka et al., 1995, Journal of Virology 69:5640-5649); and SIV isolate Mac251 from Dr. Ronald Desrosiers (Daniel et al., 1985, Science 228:1201-1204).

### Determination of the Antiviral Spectrum of MA-Targeted Compounds.

To determine their spectrum of antiviral activity, the hit compounds were evaluated in cytopathic effect (CPE) assays against a panel of viruses from different classes. Compound specificity against Japanese Encephalitis (JEV, strain 14-14-2; Nepal JEV Institute), Yellow Fever (YFV, strain 17-D; USAMRIID), Chikungunya, (CHIKV, strain 181-25; United States Army Medical Research Institute for Infectious Disease, USAMRIID), Dengue-2 (DENV2, strain New Guinea C; University of Texas Medical Branch, UTMB), Dengue-1 (DENV1, strain TH-S-MAN; UTMB), Dengue-3 (DENV3, strain H87; UTMB), Respiratory Syncytial Virus (RSV, strain A2; Functional Genetics), Vaccinia (VACCV, strain NYCBH; USAMRIID), Dengue-4 (DENV4, strain H241; UTMB), Influenza H1N1 (INFV, strain A/PR/68; Charles River Labs) was assessed at Integrated Biotherapeutics, Inc. (Gaithersburg, Md.).

Briefly, Vero cells (for viruses DENV, JEV, RSV, CHIKV, and YFV), BSC-40 cells (virus VACCV), or Madin-Darby Canine Kidney cells (virus INFV) were seeded in 96-well plates at 10⁴ cells per well in Dulbecco's modified minimal essential medium (virus VACCV), minimal essential medium (virus DENV, JEV, RSV, CHIKV, and YFV), or UltraMDCK (virus INFV, supplemented with 1 μg/ml TPCK-treated trypsin), containing 2 mM L-glutamine, 100 units/ml penicillin, 100 μg/ml streptomycin, and Fetal bovine serum (FBS, Invitrogen; 5% FBS for VACCV, 1% FBS for JEV, YFV, DENV, RSV, and 0% for INFV). Cells were incubated at 37° C. in humid incubator containing 5% CO₂. Dose-response curves were generated by measuring CPE at a range of compound concentrations. Eight compound concentrations (100, 50, 25, 12.5, 6.25, 3.13, 1.56, and 0.78 μM) were used to generate inhibition curves suitable for calculating the ECso from virus-induced CPEs. Compound dilutions were prepared in DMSO prior to addition to the cell culture medium. The final DMSO concentrations in all samples were 0.1%. Cells were infected with approximately 0.1 plaque-forming units (PFU) per cell approximately 1 hour after addition of compound. At a virus-dependent 4-6 days post-infection, cultures were fixed with 5% glutaraldehyde and stained with 0.1% crystal violet in 5% methanol. Virus-induced CPE was quantified spectro-photometrically by absorbance at 570 nm. ECsos were calculated by fitting the data to a four-parameter logistic model to generate a dose-response curve using XLfit 5.2 (equation 205, IBDS, Emeryville, Calif.). The linear correlation coefficient squared (R²) for fitting data to this model was typically >0.98%. From this curve, the concentration of compound that inhibited virus-induced CPE by 50% was calculated. As controls, uninfected cells and cells receiving virus without compound were included on each assay plate, as well as the reference agent Ribavirin (Sigma) when applicable.

The effects of the compounds on HSV-1 (dsDNA; strain HF evaluated in Vero cells; virus and cells obtained from the American Type Culture Collection [ATCC]) were assessed as described previously (Ptak et al., 2008, Antimicrob Agents Chemother 52:1302-1317).

### Example 1

### Selection of a Conserved Binding Pocket on HIV-1 MA

The amino acid sequences of the matrix domains from a subset of the available HIV-1 Pr55Gag proteins present within the National Center for Biotechnology Information Entrez Protein database (˜20,000 sequences from a total of 50,133) were analyzed. The matrix protein sequences were analyzed using BLAST (Basic Local Alignment Search Tool) (Altschul et al., 1990, J. Mol. Biol. 215:403-10) and the regions on the protein surface were identified and assessed according to the level of the sequence conservation (FIG. 12). As illustrated in FIG. 12, the amino acids present at the surface of the MA protein show a wide degree of variation, ranging from less than 20% conservation to complete conservation. Many of the observed polymorphisms are conservative and do not change the hydrophobic character, polarity, or charge of the site; however, they do change the volume and geometry of the surface. Such conservative mutations are often associated with drug resistance as the mutant protein still needs to bind to its natural targets to perform its function (Todd et al., 2000, Biochemistry 39:11876-83 and Velazquez-Campoy et al., 2003, Curr. Drug Targets Infect. Disord. 3:311-28). Interestingly, a recess that runs across the globular head of MA shows remarkable conservation (FIG. 12). The conservation of the area suggests a high degree of functional and/or structural importance. Indeed, this site overlaps the PI(4,5)P₂ binding site that is responsible for the high-affinity interaction of the Gag protein with membranes, a critical function required for the correct targeting and assembly of infectious virions. Moreover, the predicted binding site for the small-molecule antiviral ITI-367 also inhabits a portion of this recess. As low-molecular-weight inhibitors should preferably not depend on interactions with variable regions in order to maintain high binding affinity against multiple targets, this conserved region was selected to focus our efforts to discover MTI compounds.

### Example 2

### Preparation of Three-Dimensional Models of MA for Docking

An expanded high throughput computer docking (HTCD)-based strategy was developed to identify novel MA binders. This strategy used models generated using available NMR and X-ray structures of MA and a large initial diversity library, and allowed the identification of potential hits using strict criteria based on predicted affinity and physical-chemical properties.

The 2GOL x-ray structure of HIV-1 MA (Kelly et al., 2006, Biochemistry 45(38): 11257-11266) and 2H3Z NMR structure of HIV-1 MA (Saad et al., 2006, Proc. Natl. Acad. Sci. U.S.A. 103(30):11364-11369) were retrieved from RCSB Protein Data Bank for molecular modeling. The complex 2GOL was selected on the basis of its high resolution (2.2 Å) and 2H3Z was selected because it contains a ligand in the binding site.

Prior to molecular modeling the capsid protein p24 (CA) was removed from the 2GOL structure, and di-C4-phosphatidylinositol-(4,5)-biophosphate (di-C4-PI[4,5]P₂) was removed from the 2H3Z structure.

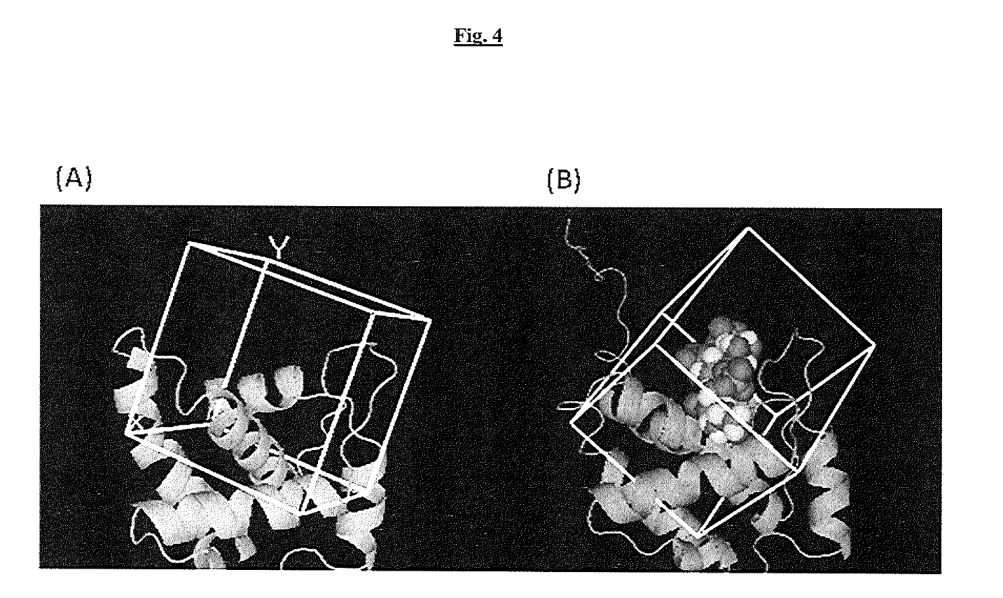

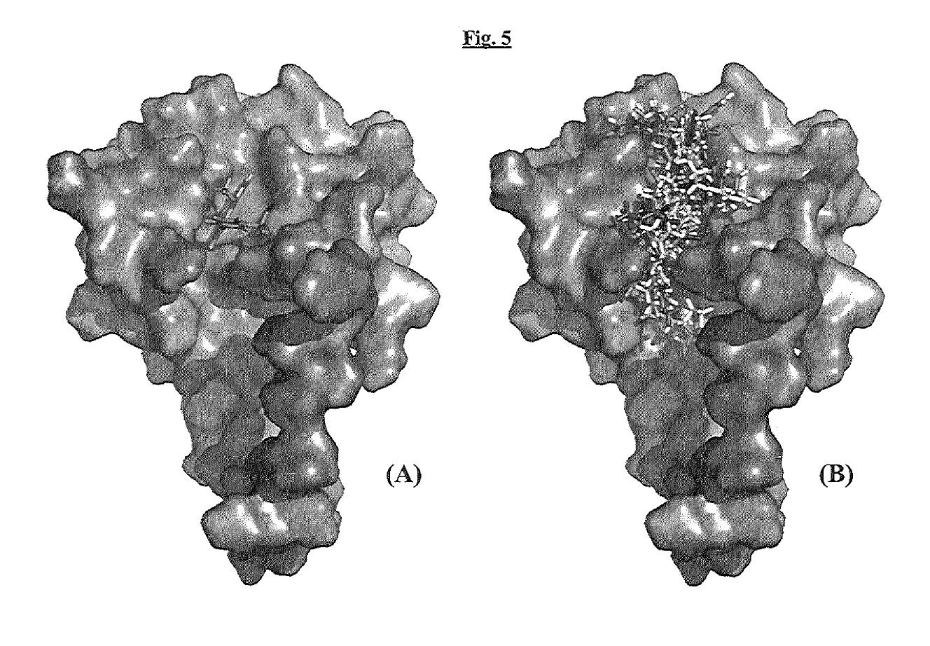

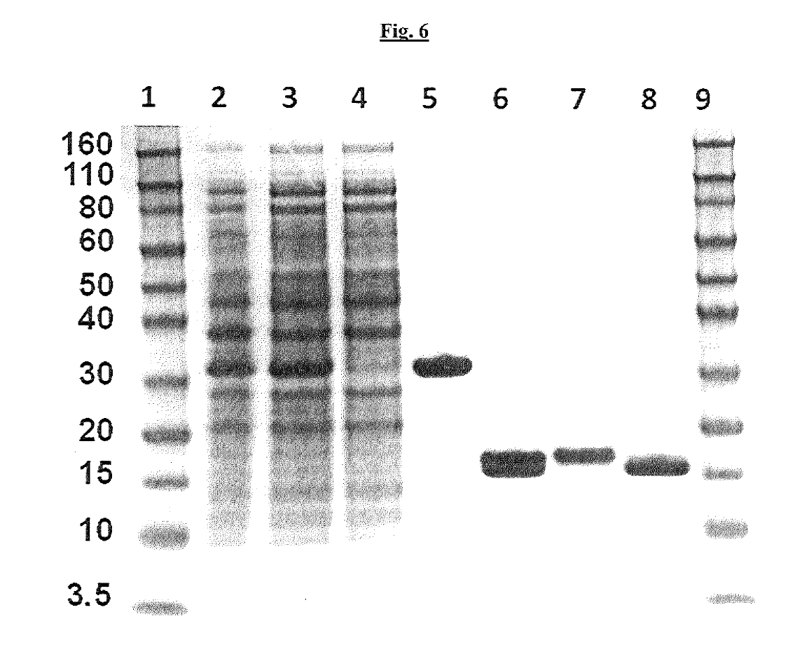

The docking area was restricted by a box of 20 Å×20 Å×20 Å on the 2GOL structure and 19 Å×20 Å×21 Å on the 2H3Z structure (FIGS. 4A and 4B). This region contained the proposed binding site for the anti-HIV compound ITI-367 (3-(2-methoxyphenyl)-4-[3-trifluoronethyl-phenyl]-1,2,4-oxadiazol-5-one; Haffar et al., 2005, J. Virol. 79(20):13028-13036) and also encompassed the di-C4-PI(4,5)P₂ binding pocket of MA.

### Example 3

### Validation of 3-Dimensional Models of MA for Docking

ITI-367 (3-(2-methoxyphenyl)-4-[3-trifluoromethyl-phenyl]-1,2,4-oxadiazol-5-one; Haffar et al., 2005, J. Virol. 79(20):13028-13036) was docked using QUANTUM molecular modeling software (QuantumLead, Quantum Pharmaceuticals, Moscow, Russia) to the two models of the HIV-1 MA protein, in order to determine the small molecule binding mode and compare it with published results.

Two three-dimensional structure models of HIV-1 MA obtained by the two different experimental techniques (x-ray and NMR) were used for docking to compare the results and exclude possible structural artifacts caused by these techniques. FIG. 5 shows the docked position of ITI-367 in the binding pocket of MA in 2H3Z and 2GOL structures (panels a and b, respectively).

Despite differences in the two protein structures obtained by x-ray and NMR, the docked positions of ITI-367 were quite similar. The results of the docking were thus found to be independent of the experimental technique used to obtain the three-dimensional structure model of MA, suggesting that predicted position of the ligand in the binding site reflected its true force-field and was unaffected by possible structure artifacts caused by x-ray or NMR. This predicted position was in good agreement with the published docking site prediction and demonstrated the ability to computationally dock small molecules to our derived models. The predicted K_{D} based upon our model for the ITI-367-MA interaction is approximately 10 mM.

### Example 4

### High-Throughput Combinatorial Docking of Screening Collection Against Structural Models of the HIV-1 MA Protein

High-throughput combinatorial docking experiments were performed with a chemically diverse proprietary screening collection composed of approximately 1,168,625 small organic compounds.

The virtual screening procedure included two stages: (1) docking to a static protein model and (2) refinement using a dynamic protein model. These two procedures were performed using QUANTUM software utilities. Docking to a static protein model included identification of the ligand position in the binding pocket with the minimal binding energy, and its estimation. In the second stage, the binding energies for hits were refined with regard to the protein flexibility using molecular dynamics. The refinement procedure was a complete free energy perturbation molecular dynamics run for the whole protein-ligand complex in aqueous environment. Thus, it considered both protein and ligand flexibility. In addition, the calculation of physical-chemical properties of small molecules was performed using Q-Mol ver. 1.0.6 (Quantum Pharmaceuticals). A compound was regarded as a hit if the predicted affinity (Ku) was in the range 1 to 10 μM as well as displaying favorable, drug-like chemical characteristics.

Docking of the screening collection to the static MA model yielded no compounds with a predicted K_{D} below 1 μM, and 29 compounds with the K_{D} ranging from 1 to 10 μM. These compounds were sent to the refinement procedure.

Table 1 shows ID and refined K_{D} for hit compounds. As a result, four compounds were identified as having K_{D} values less than 1 μM, and fifteen compounds were identified as having K_{D} values ranging from 1 μM up to 10 μM. In addition, Table 1 shows the contributions of the different types of interactions to the free binding energy of the hits. The predicted positions of all of the hits in a structural groove within HIV-1 MA are shown in FIG. 6A, and the position of the compound with the highest predicted binding energy (CMPD-1) with HIV-1 MA is shown in FIG. 6B.

TABLE 1
Predicted affinities of potential hits identified using virtual
screening against MA and contributions of different interactions
to the free binding energy of the protein-ligand complex.

| | Predicted | Binding energies, kJ/mol | | | | |
|---|---|---|---|---|---|---|
| Compound | K_{D}, μM | E_{bind} | Eᵢₙₜ | Eₚₒₗₐᵣ | Eₙₒₙₚₒₗₐᵣ | TΔS |
| CMPD-1 | 0.3 | −37.7 | 55.4 | −67.9 | −192.6 | −167.5 |
| CMPD-2 | 0.4 | −36.5 | 2.4 | 122.7 | −218.4 | −56.8 |
| CMPD-3 | 0.7 | −35.3 | 9.4 | 127.8 | −214.3 | −41.7 |
| CMPD-4 | 0.9 | −34.8 | 55.7 | −15.9 | −196.6 | −122.1 |
| CMPD-5 | 1.0 | −33.9 | 16.3 | 65.2 | −194.4 | −79.0 |
| CMPD-6 | 2.0 | −33.2 | 13.4 | −10.7 | −171.7 | −135.8 |
| CMPD-7 | 2.0 | −33.1 | 32.6 | 61.8 | −196.7 | −69.1 |
| CMPD-8 | 2.0 | −32.4 | 25.6 | 76.8 | −198.4 | −63.6 |
| CMPD-9 | 2.0 | −32.4 | 35.1 | −15.4 | −173.2 | −121.2 |
| CMPD-10 | 3.0 | −31.9 | 4.9 | 74.6 | −184.5 | −73.0 |
| CMPD-11 | 3.0 | −31.8 | 18.9 | 118.0 | −198.3 | −29.5 |
| CMPD-12 | 4.0 | −31.1 | −11.5 | 143.1 | −193.8 | −31.1 |
| CMPD-13 | 5.0 | −30.4 | 13.4 | 97.8 | −188.6 | −47.0 |
| CMPD-14 | 6.0 | −29.9 | −11.0 | 3.2 | −148.4 | −126.4 |
| CMPD-15 | 8.0 | −29.3 | −8.3 | 16.9 | −151.9 | −114.1 |
| CMPD-16 | 9.0 | −29.0 | −15.5 | 106.2 | −168.6 | −48.9 |
| CMPD-17 | 9.0 | −28.9 | 17.4 | 111.9 | −186.0 | −27.7 |
| CMPD-18 | 10.0 | −28.6 | 6.4 | 86.6 | −169.3 | −47.7 |
| CMPD-19 | 10.0 | −28.5 | 32.9 | −1.4 | −158.1 | −98.0 |

E_{bind}: free binding energy of the protein-ligand complex;
Eᵢₙₜ: energy of bonds stretching, bending, and torsion;
Eₚₒₗₐᵣ: Coulomb energy of interacting charges, plus electrostatic interaction with the surrounding water molecules (electrostatic contribution to solvation energy);
Eₙₒₙₚₒₗₐᵣ: van der Waals energy, plus short-range interaction with the surrounding water molecules (non-polar contribution to the solvation energy);
TΔS: entropy contribution.

The studies described identified nineteen compounds with predicted affinities for the HIV-1 MA protein from 0.3 μM to 10 μM that bind along a recess that runs across the globular head of MA and encompasses the PI(4,5)P₂ binding site. The predicted binding site for ITI-367 also comprises a portion of this recess, close to the putative nuclear localization signal. However, the compounds identified extend further down this structural groove towards the region of the protein responsible for other functions of the protein such as envelope incorporation (FIG. 7).

Compound biologic activity also depends on bioavailability, which is greatly affected by physical-chemical properties. Therefore, parameters such as log P and solubility were calculated for the compounds of interest (Table 2).

As shown on Table 2, the predicted log P values for the hits range from −0.3 to 4.6, the compounds have from three to nine H-bond donors, from zero to three H-bond acceptors, and molecular weights ranging from 366 to 478 Da. Thus, all predicted hits comply with Lipinski's “rule of 5,” promising good bioavailability.

Taking into account the predicted binding position of the compounds in a structural groove contacting points of MA known to mediate its diverse functions (a binding groove that is shared with ITI-367; FIG. 7), coupled with the high predicted affinity and predicted bioavailability, these compounds may inhibit MA function and act as anti-HIV-1 drugs.

TABLE 2
Predicted physical-chemical properties and compliance with Lipinski's rule
of 5 for the identified compounds (DMSO = dimethyl sulfoxide)

| | | Water | DMSO | | H- | H- | | |
|---|---|---|---|---|---|---|---|---|
| | MW, | sol., | sol., | Log | bond | bond | Rotatable | Lipinski's |
| Cmpd | Da | g/L | g/L | P | acceptors | donors | bonds | rule |
| CMPD-1 | 474.55 | 0 | 4.9 | 4.5 | 7 | 1 | 13 | TRUE |
| CMPD-2 | 470.54 | 0 | 1.6 | 3.6 | 9 | 1 | 7 | TRUE |
| CMPD-3 | 463.36 | 0 | 1.4 | 3.9 | 7 | 0 | 8 | TRUE |
| CMPD-4 | 453.55 | 0.002 | 1.6 | 1.5 | 8 | 2 | 13 | TRUE |
| CMPD-5 | 406.43 | 0.001 | 1.5 | 2.5 | 8 | 1 | 6 | TRUE |
| CMPD-6 | 420.89 | 0 | 0.9 | 4 | 6 | 0 | 5 | TRUE |
| CMPD-7 | 411.47 | 0.003 | 0.4 | 2.9 | 7 | 1 | 8 | TRUE |
| CMPD-8 | 467.56 | 0.002 | 0.5 | 2.2 | 9 | 2 | 12 | TRUE |
| CMPD-9 | 477.46 | 0 | 2.9 | 3.4 | 8 | 1 | 10 | TRUE |
| CMPD-10 | 468.55 | 0 | 2.1 | 3.4 | 8 | 2 | 11 | TRUE |
| CMPD-11 | 389.49 | 0.728 | 1.3 | −0.3 | 8 | 2 | 10 | TRUE |
| CMPD-12 | 478.35 | 0 | 1.6 | 3.4 | 8 | 0 | 9 | TRUE |
| CMPD-13 | 467.54 | 0 | 0.6 | 4.3 | 7 | 1 | 5 | TRUE |
| CMPD-14 | 409.48 | 0 | 4.9 | 2.4 | 7 | 2 | 11 | TRUE |
| CMPD-15 | 415.48 | 0 | 2 | 4.6 | 6 | 2 | 9 | TRUE |
| CMPD-16 | 466.35 | 0 | 2.3 | 4.5 | 5 | 1 | 6 | TRUE |
| CMPD-17 | 410.45 | 0.001 | 0.9 | 3.5 | 9 | 0 | 8 | TRUE |
| CMPD-18 | 448.56 | 0.002 | 2.2 | 2.7 | 8 | 1 | 9 | TRUE |
| CMPD-19 | 413.62 | 0 | 5 | 4.5 | 3 | 1 | 7 | TRUE |

### Example 5

### Anti-Viral Activity of Selected Compounds of the Invention

Small-molecule targeting of the HIV-1 MA protein may yield inhibitors capable of disrupting HIV-1 replication at either a post-entry or an assembly stage, or both. Therefore, a single-round infection assay was used in order to determine whether the identified compounds (a) inhibit viral replication and (b) affect early events, late events, or both. Details of the single-round infection assay have been published in detail (Rossi et al., 2008, Retrovirology 5:89; Cocklin et al., 2007, J. Virol. 81(7):3645-3648; Martin-Garcia et al., 2006, Virology 346(1):169-179; Martin-Garcia et al., 2005, J. Virol. 79(11):6703-6713).

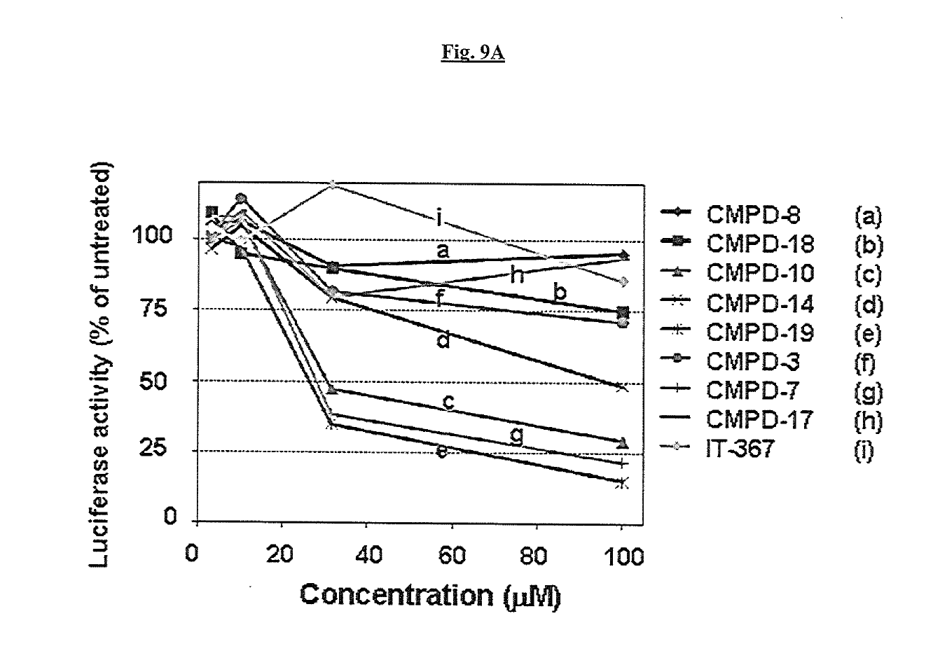

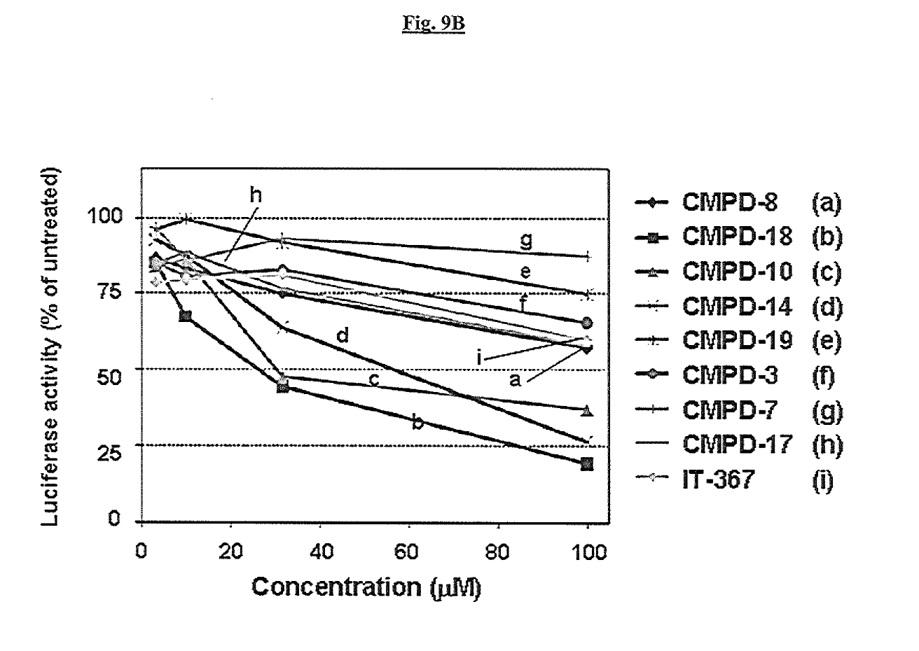

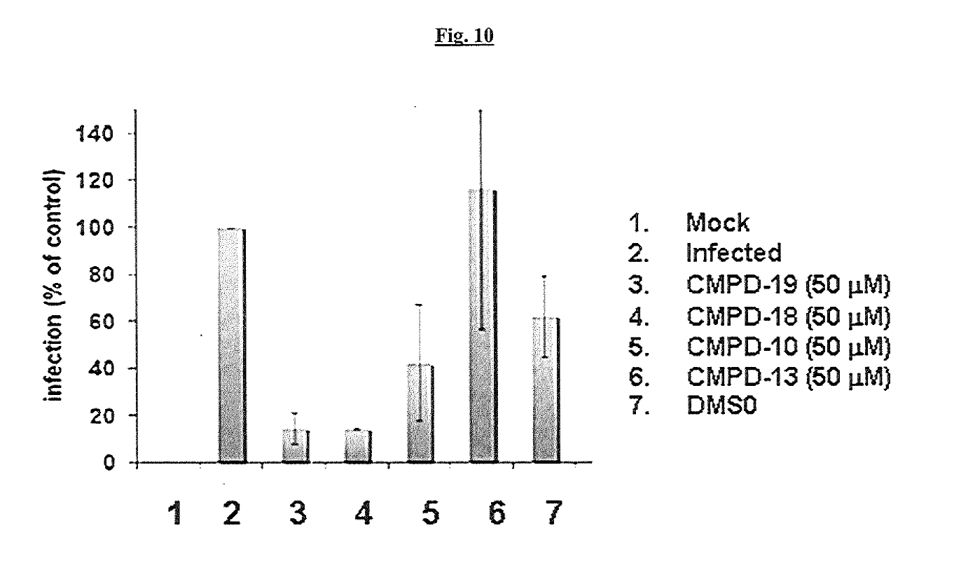

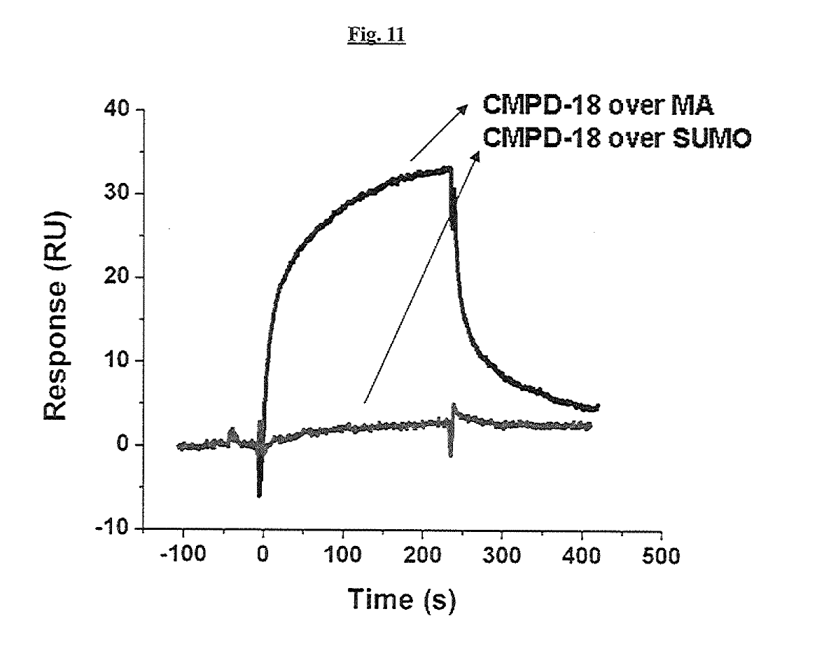

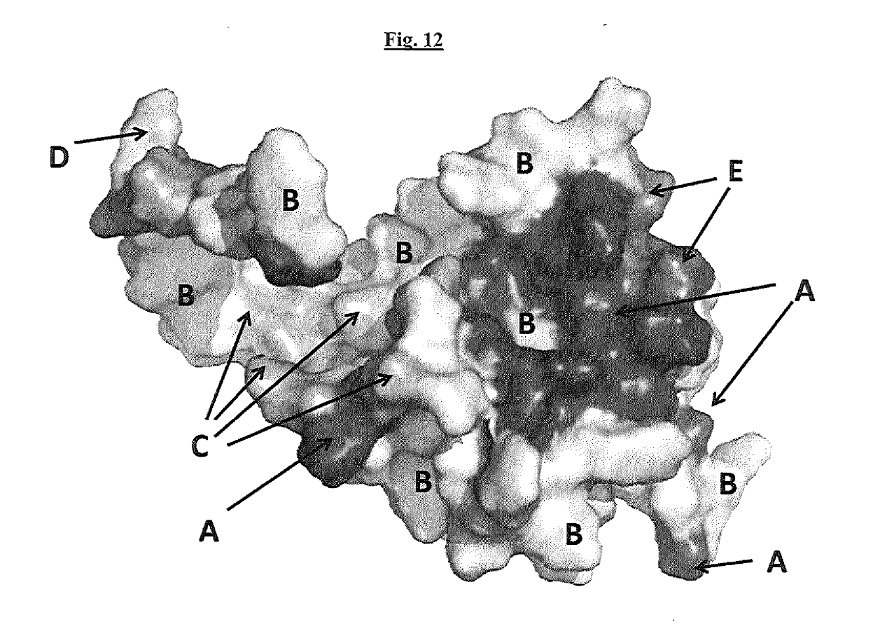

Effects on assembly were identified by incubating the viral producer cells (293T) in the absence or in the presence of various concentrations of the compounds. Supernatants containing virus (that encodes for firefly luciferase as a reporter gene) were then diluted 10-fold and used to infect the target cells (U87 CD4-CXCR4). Compound-induced aberrant assembly was then manifested as a decrease in infectivity of the target cells, as compared to those infected with virus from untreated cells. Complementarily, effects on early-stage events were determined by producing virus in the absence of compound, then exposing target cells to virus in the absence or presence of various concentrations of compounds. From this analysis we have determined that compounds CMPD-17, CMPD-8, CMPD-3, CMPD-10, CMPD-14, and CMPD-18 affect viral replication at an early, pre-integration event (FIG. 9B); compounds CMPD-10, CMPD-14, CMPD-19 and CMPD-7 disrupt the production of infectious virus (FIG. 9B); and compounds CMPD-10 and CMPD-14 have the ability to disrupt viral replication at both early and late stages (FIGS. 9A and 9B). The previously discovered small molecule matrix inhibitor ITI-367 ((3-(2-methoxyphenyl)-4-[3-trifluoromethyl-phenyl]-1,2,4-oxadiazol-5-one) was included in this analysis for comparison. Not only do the compounds of the invention show increased efficacy over ITI-367 but these small-molecule inhibitors are capable of disrupting both early- and late-stage processes in the replication cycle of HIV-1. Calculated 50% inhibitory concentration (IC₅₀) values are shown in Table 3.

TABLE 3
IC₅₀values of MTI compounds derived from single-round
infection assays

| | IC₅₀ early stages | IC₅₀ late stages |
|---|---|---|
| Compound | (μM) | (μM) |
| CMPD-8 | >100 | Not Active |
| CMPD-18 | 26 | >100 |
| CMPD-10 | 30 | 31 |
| CMPD-14 | 57 | 95 |
| CMPD-19 | Not Active | 26 |
| CMPD-3 | >100 | >100 |
| CMPD-7 | Not Active | 28 |
| CMPD-17 | >100 | Not Active |
| ITI-367 | >100 | Not Active |

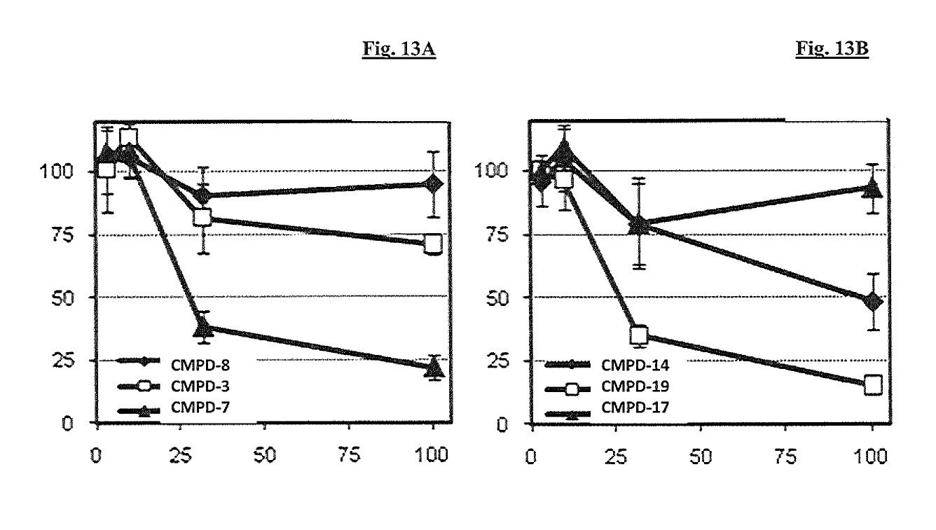

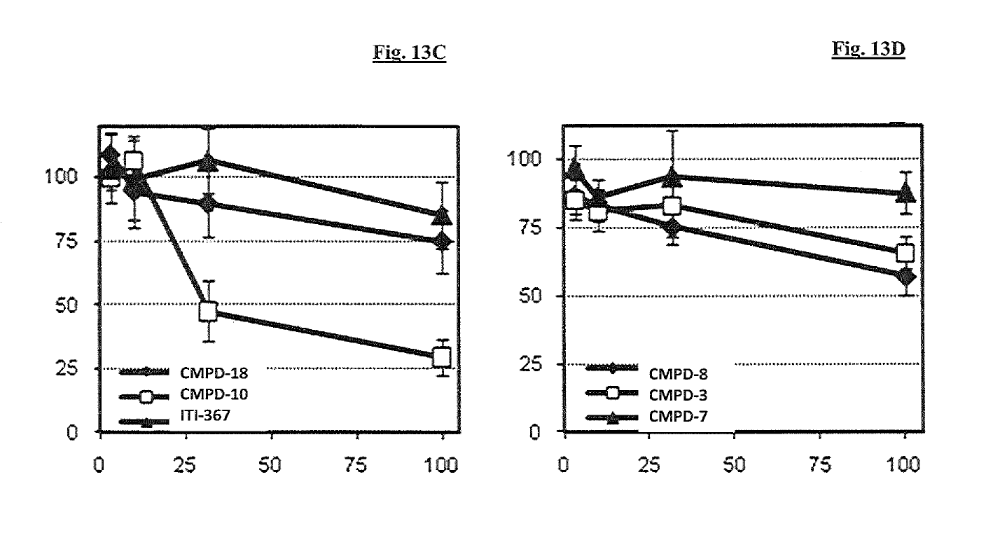

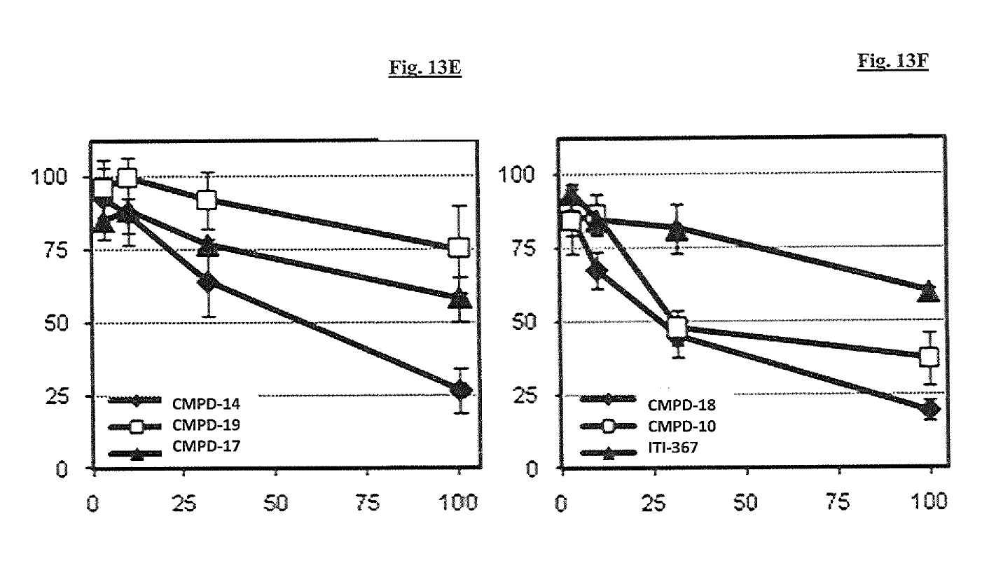

The cytotoxicity of the compounds on the producer cells and the target cells was evaluated by measuring the release of the cellular enzyme lactate dehydrogenase (LDH) into the culture supernatants. The potential cytotoxicity of the compounds either on the producer cells or on the target cells was evaluated by measuring the release of the cellular enzyme lactate dehydrogenase (LDH) into the culture supernatants. Although some of the 19 compounds evaluated induced cytotoxicity on producer and/or target cells, none of the eight compounds with antiviral activity shown in FIG. 13 promoted LDH release from either treated producer or treated target cells in significantly higher amounts than those observed in the corresponding untreated cells with medium alone or with medium containing the same DMSO concentration present in the cells exposed to each compound concentration (50% Toxic Concentration [TC₅₀]>100 μM). Only a slight increase in LDH release in U87 cells exposed to 1 mM (ten-fold higher than the highest concentration used for functional studies) of compounds MTI487, MT1293 and MTI164 was observed over that observed in cells exposed to the same concentration of DMSO without compound (data not shown).

### Example 6

### Compounds Representative of Early-Stage, Late-Stage and Dual-Stage MA-Targeted Inhibitors Reduce Replication of Fully Infectious HIV-1

Although production of pseudovirions by transfection and the ability to analyze inhibition in a single round are advantageous for addressing the inhibitory effect of a given compound, the single-round infection assays cannot address the effects of multiple rounds of infection and cell-to-cell spread on the efficacy of the test compounds. Moreover, the producer and target cell lines used are not representative of the natural targets of HIV-1 infection. Therefore, the question of whether or not the MTI compounds identified in the single round infection assays still retained activity against fully infectious virus (HIV-1 IIIB) replicating in a T-cell derived cell line (Sup-T1) was addressed.

In this experiment (FIG. 10), HIV-1 replication was inhibited in the presence of CMPD-19, which was characterized in single-round assays as a late-stage inhibitor, or CMPD-18, which was shown to be an early-stage inhibitor. CMPD-10, which was characterized previously as a dual-stage inhibitor, also inhibited HIV-1 replication, albeit to a lesser extent relative to the single-stage inhibitors. In contrast, the compound CMPD-13, which had no activity in the pseudovirus assays, similarly had no effect on infectious HIV-1 replication. The results of this experiment, which corroborate the findings of the pseudovirus assays (FIG. 10), demonstrated inhibition of infectious, replication-competent virus by compounds hypothesized to interact with HIV-1 matrix (MA).

### Example 7

### MA-Targeted Inhibitors Reduce Replication of Fully Infectious HIV-1 in Peripheral Blood Mononuclear Cells

The question of whether the MTI compounds identified in the single-round infection assays retained activity against fully infectious, primary virus isolates replicating in primary human peripheral blood mononuclear cells (PBMCs) was also investigated. Moreover, the specificity of the MTI compounds to HIV-1 was evaluated. Therefore, the eight compounds identified from the single-round infection assay were assayed against the clinical isolates HIV-1_{92BR030} (Subtype B) and HIV-1_{93IN101} (Subtype C), as well as against SIV_{mac251} in PBMCs. Four of the eight compounds inhibited HIV-1_{92BR030} and HIV-1_{93IN101}, with IC₅₀ values in the range of 9 μM to 34 μM (Table 4). Furthermore, the compounds also inhibited SIV_{mac251} with similar IC₅₀ values. These experiments identified CMPD-18 (N-[4-ethoxy-3-(piperidin-1-ylsulfonyl)phenyl]-2-imidazo[2,1-b][1,3]thiazol-6-ylacetamide); CMPD-7 (2-[4-[[3-(4-fluorophenyl)-1,2,4-oxadiazol-5-yl]methyl]piperazin-1-yl]-N-(p-tolyl)acetamide); CMPD-14 (N-2-(phenoxyacetyl)-N-[4-(piperidin-1-ylcarbonyl)benzyl]glycinamide); and CMPD-17 (3-(2-ethoxyphenyl)-5-[[4-(4-nitrophenyl)piperazin-1-yl]methyl]-1,2,4-oxadiazole) as compounds for further study (structures provided in Table 4),

TABLE 4
Antiviral Activity of the MTI compounds against HIV-1 and SIV in PBMCs

| | | | | | Antiviral |
|---|---|---|---|---|---|
| | Virus | IC₅₀ | IC₉₀ | TC₅₀ | index |
| Compound | Isolate | (μM) | (μM) | (μM) | (TC₅₀/IC₅₀) |
| 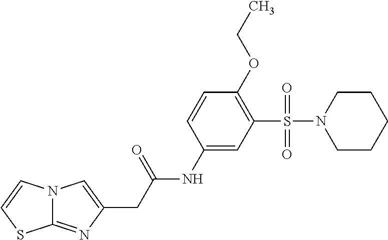 [SMILES: CCOc1ccc(NC(=O)Cc2cn3ccsc3n2)cc1S(=O)(=O)N1CCCCC1] CMPD-18 | HIV-1 92BR030 HIV-1 93IN101 SIV MAC251 | 23.4 30.8 87.1 | 94.0 85.2 >100 | 92.3 | 3.95 3.00 1.06 |
| 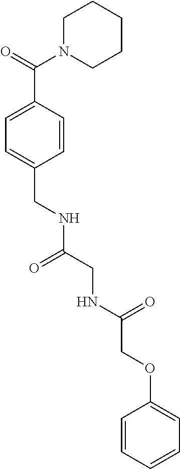 [SMILES: O=C(CNC(=O)COc1ccccc1)NCc1ccc(C(=O)N2CCCCC2)cc1] CMPD-14 | HIV-1 92BR030 HIV-1 93IN101 SIV MAC251 | 22.2 18.0 17.1 | 68.6 62.1 44.2 | >100 | >4.51 >5.56 >5.85 |
| 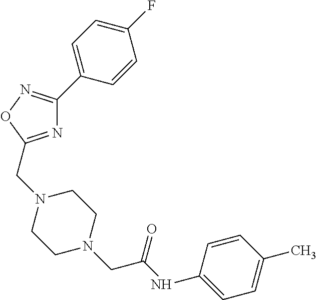 [SMILES: Cc1ccc(NC(=O)CN2CCN(Cc3nc(-c4ccc(F)cc4)no3)CC2)cc1] CMPD-7 | HIV-1 92BR030 HIV-1 93IN101 SIV MAC251 | 9.33 16.3 9.48 | >100 >100 31.0 | >100 | >10.72 >6.13 >10.55 |
| 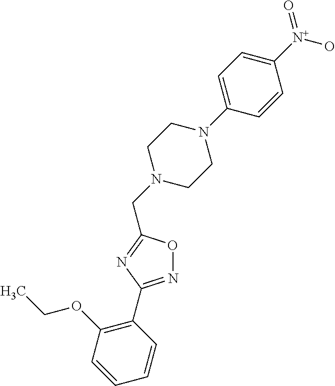 CMPD-17 | HIV-1 92BR030 HIV-1 93IN101 SIV MAC251 | 34.1 25.5 23.4 | >100 >100 >100 | >100 | >2.94 >3.92 >4.28 |
| ITI-367 | HIV-1 | 47.0 | >100 | >100 | >2.13 |
| | 92BR030 | | | | |
| | HIV-1 | 41.9 | 96.0 | | >2.39 |
| | 93IN101 | | | | |
| | SIV | 74.9 | >100 | | >1.33 |
| | MAC251 | | | | |

### Example 8

### Intercation of Compounds Possessing Antiviral Activity (As Evaluated in Single- and Multiple-Round Infection Assays) with HIV-1 MA

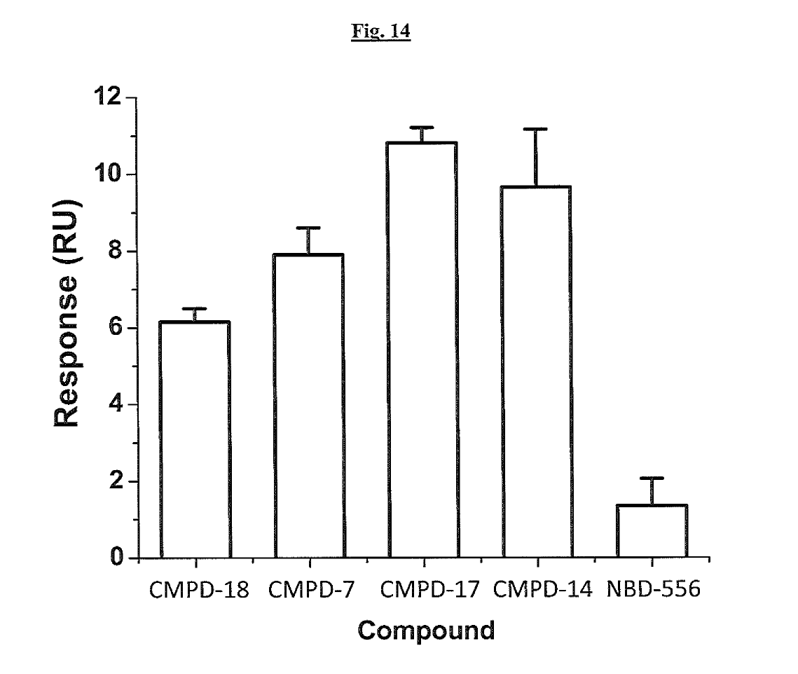

The MTI compounds are predicted to interact with HIV-1 MA and thereby disrupt its functioning during the HIV-1 replication cycle. However, it is possible that a compound might exert its action via another mechanism not involving MA. Therefore, studies were performed to determine that the MTI compounds are directed against HIV-1 MA. The direct interaction of compounds CMPD-18, CMPD-17, CMPD-7, and CMPD-14 with HIV-1 MA was assayed using surface plasmon resonance interaction analyses. Wild-type HIV-1 MA protein was overproduced in E. coli using the pETHSUL expression system and purified using successive IMAC strategies, followed by an ion exchange chromatographic step as outlined in Materials and Methods. FIG. 6 illustrates the purification profile of HIV-1 MA. The purified MA was then immobilized onto the surface of a high-capacity CM7 sensor chip. Similarly, the monoclonal antibody ARC4033 (antimouse/rat interferon-γ; BioSource, Invitrogen, Carlsbad, Calif.) was immobilized on a CM7 sensor chip and was used to correct for background binding and instrument and buffer artifacts. As illustrated in FIG. 14, the MTI compounds all directly interacted with sensor chip-immobilized HIV-1 MA. Moreover, the small-molecule CD4 mimetic compound NBD-556 displayed no such interaction with HIV-1 MA, establishing the specificity of MTI compounds for HIV-1 MA (FIG. 14).

### Example 9

### Mediation of Antiviral Effect Through MA

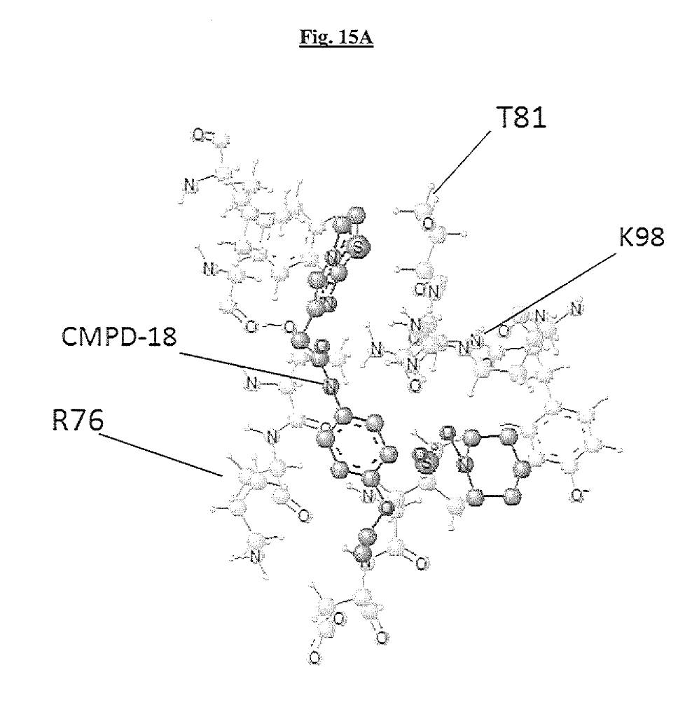

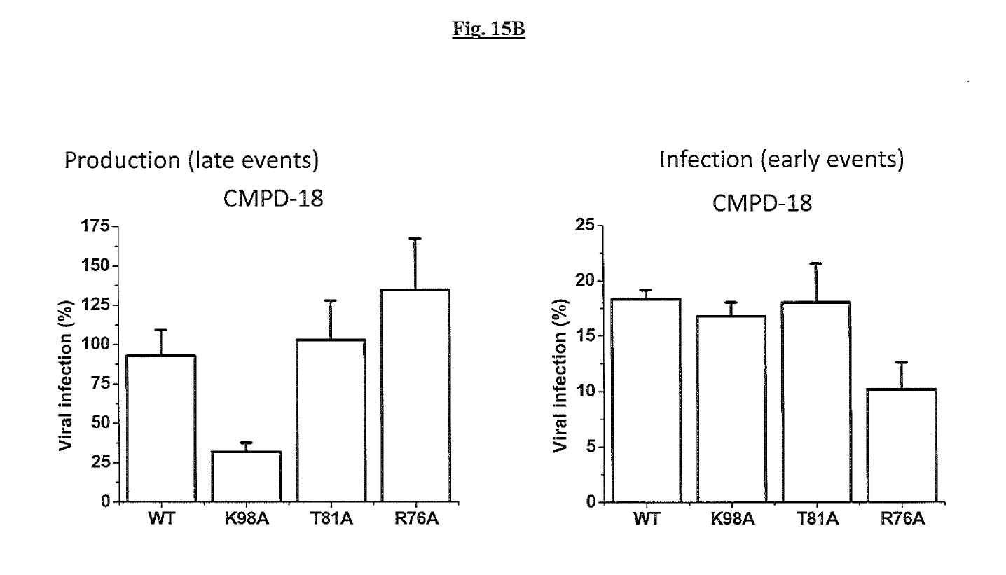

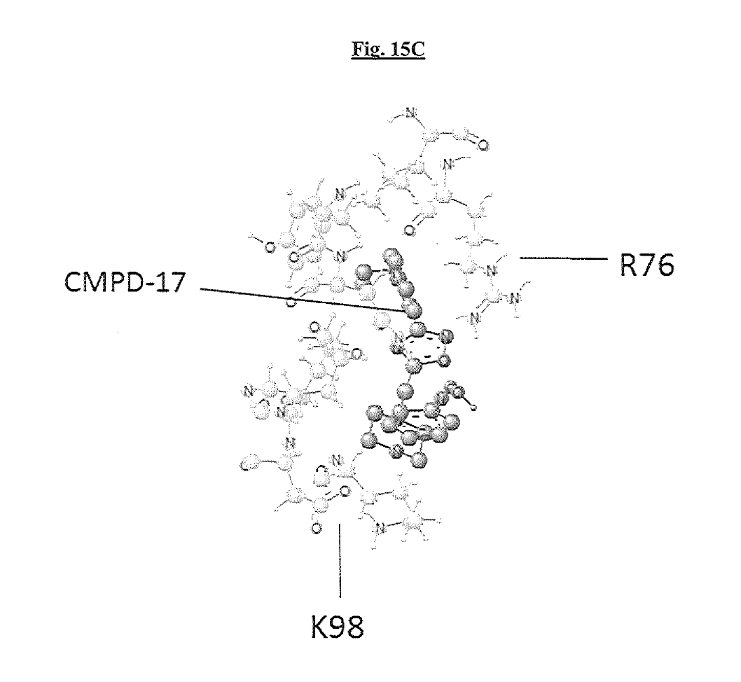

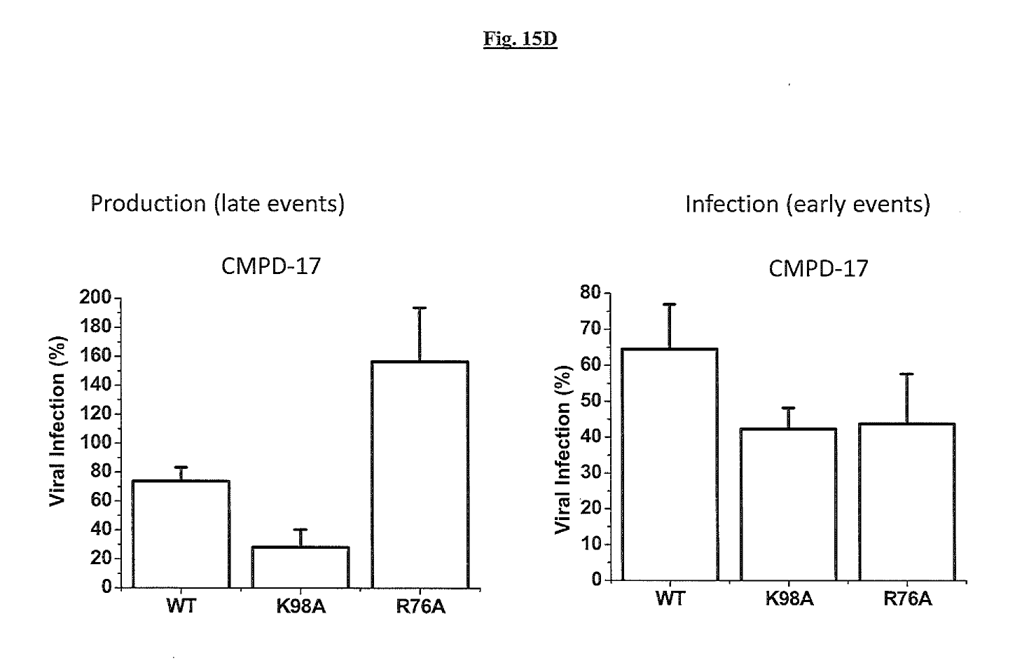

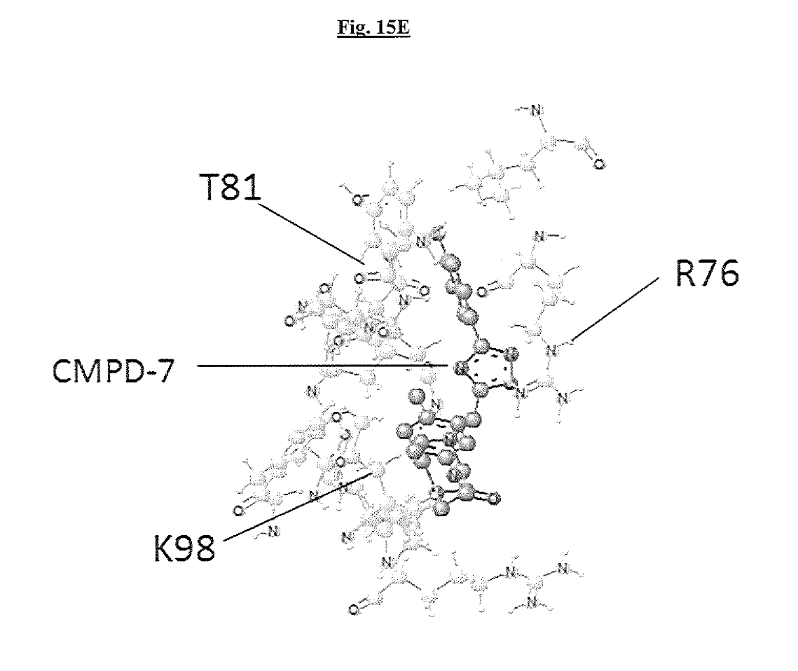

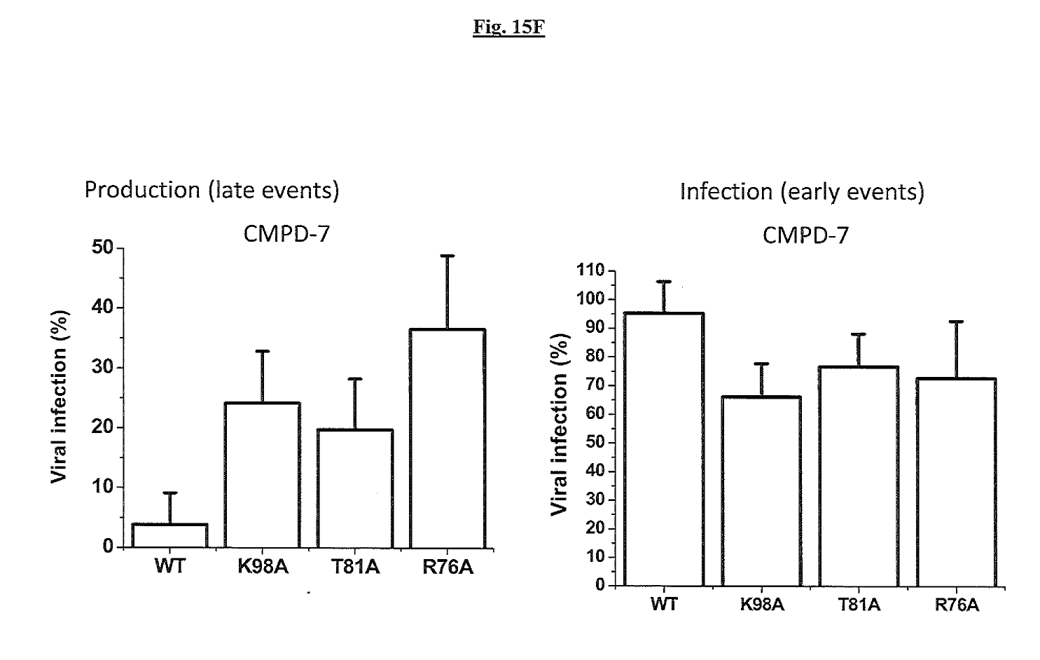

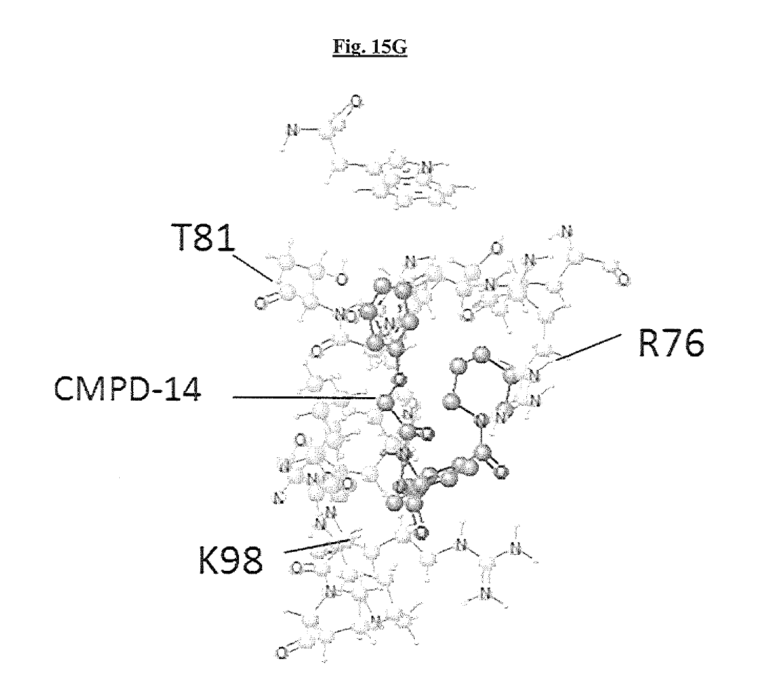

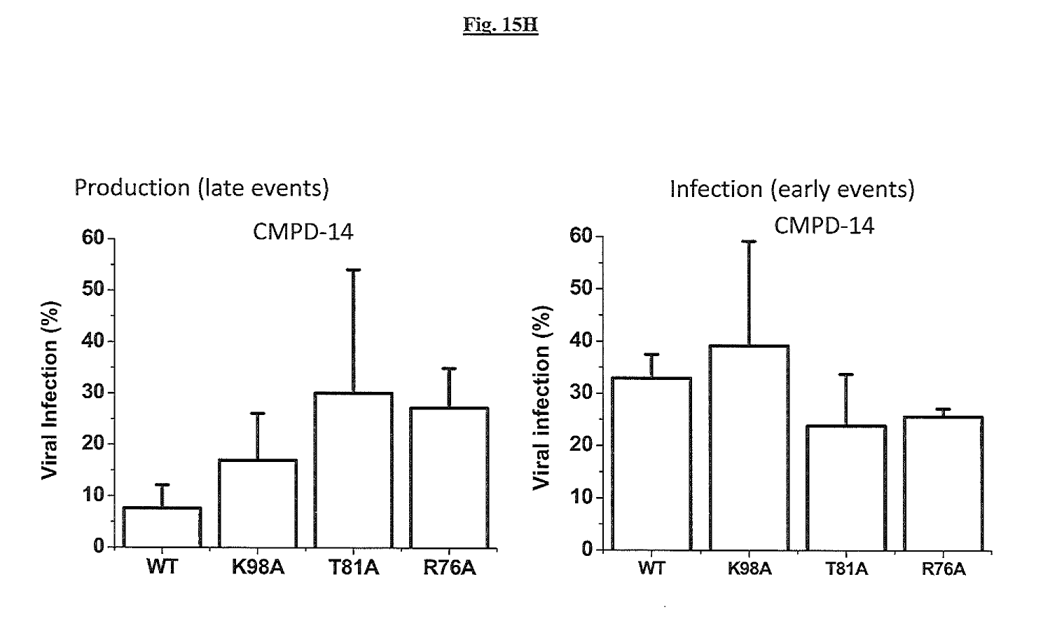

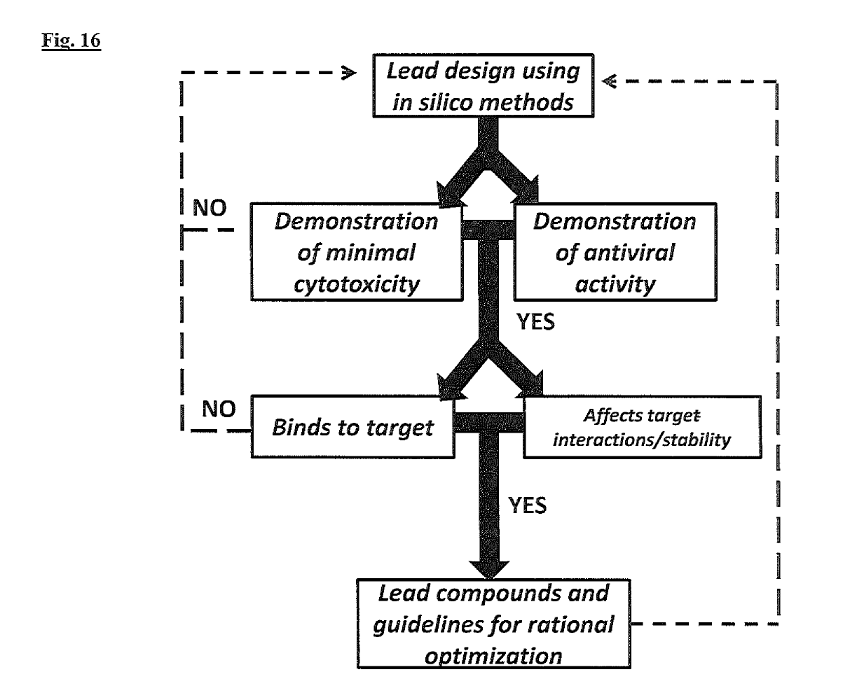

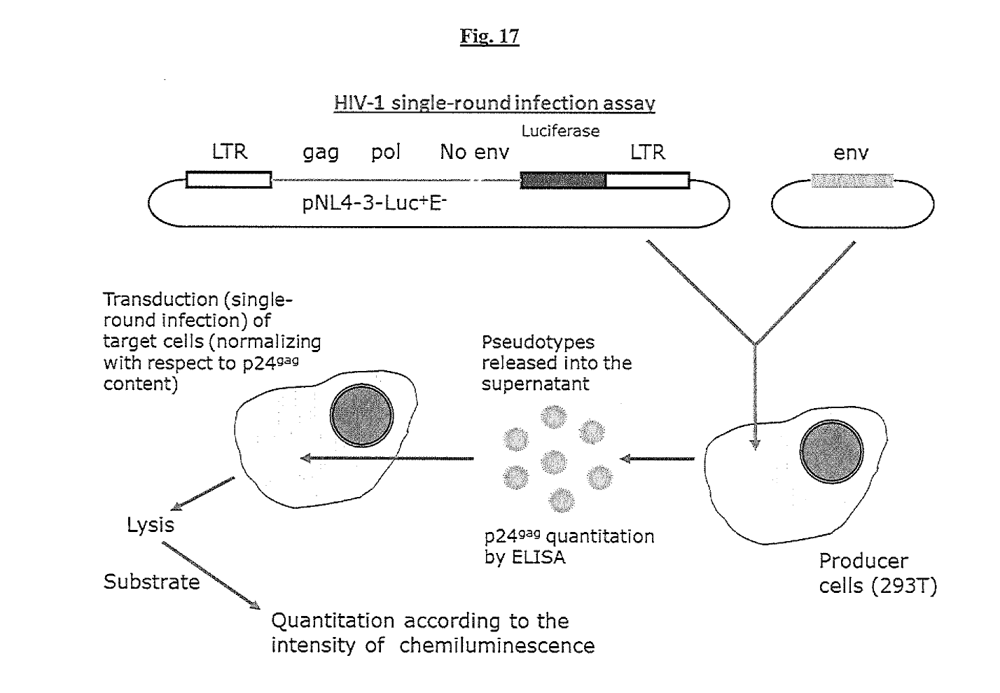

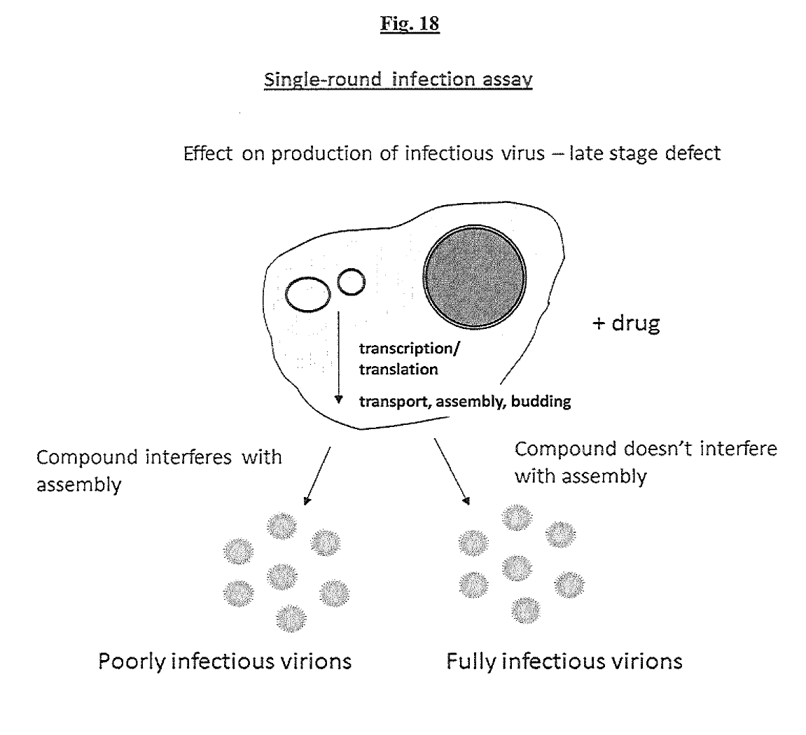

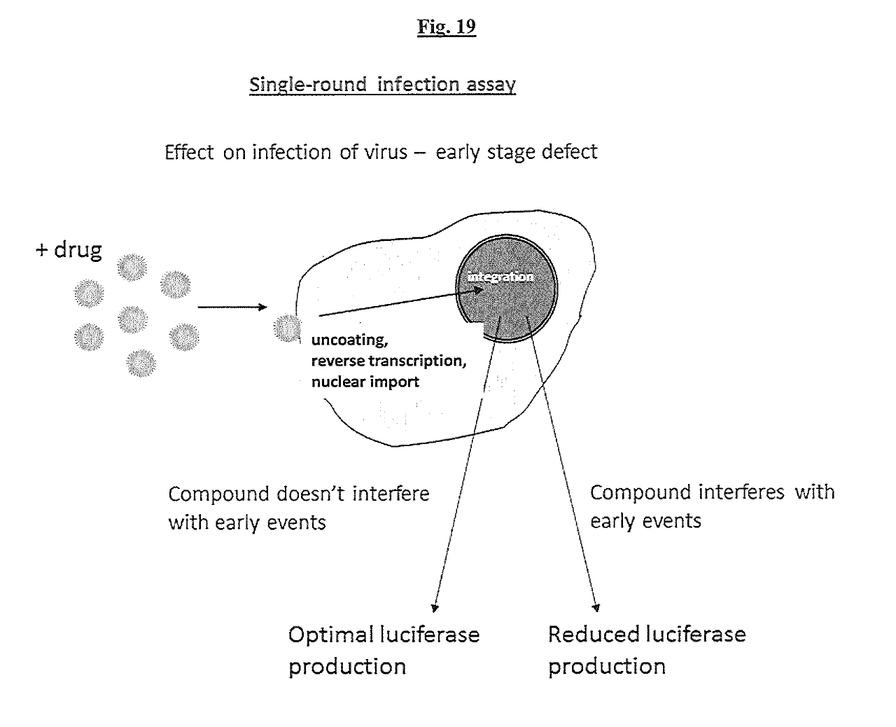

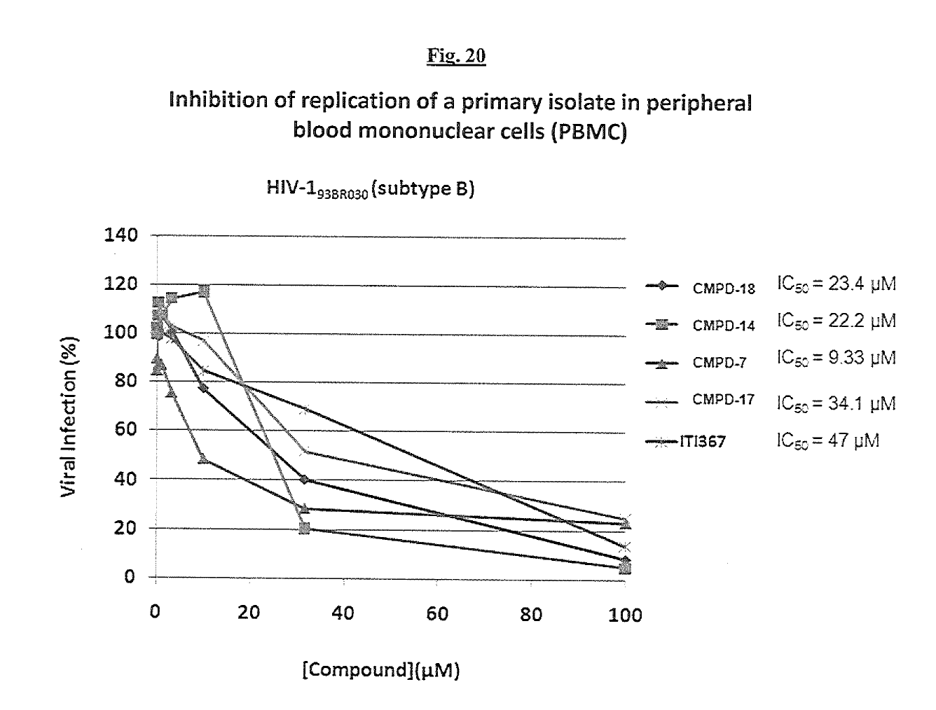

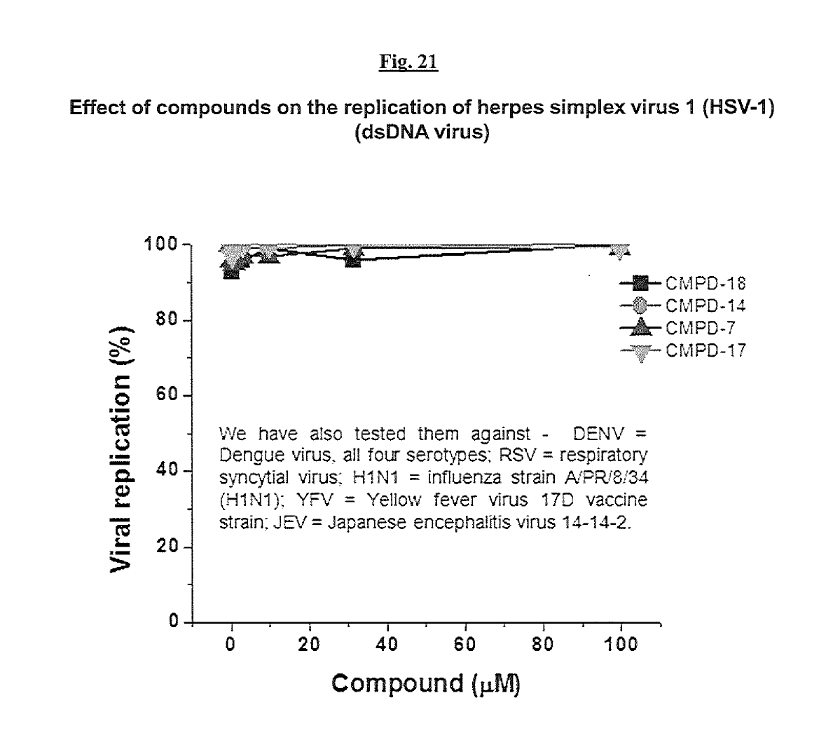

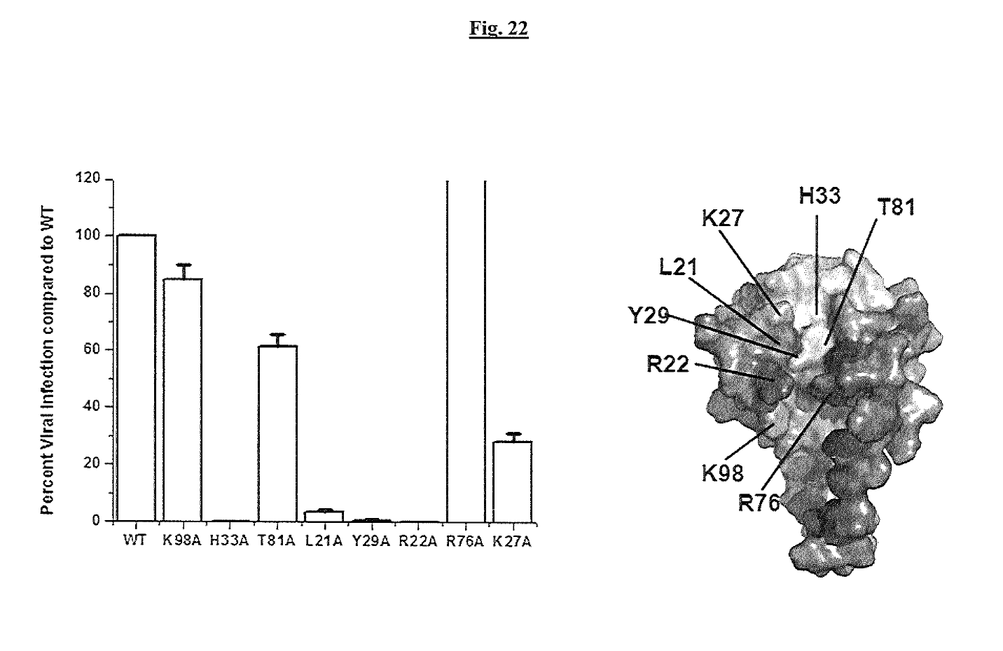

Although a direct interaction of the MTI compounds with HIV-1 MA was demonstrated, it is possible that the MTI compounds exert their antiviral effect via another mechanism. Therefore the efficacy of the MTI compounds as affected by mutation of MA was evaluated. To test this hypothesis and to provide evidence for the proposed binding site of the compounds, mutations in the MA region of the pNL4-3-LucR⁺E⁻ vector used within the single-round infection assay were created. Based on their proximity to the compounds in the docked models, seven different single-mutant clones were made. The mutations introduced into these clones were L21A, R22A, Y29A, H33A, R76A, T81A, or K98A. As these mutations are located in a highly conserved pocket within MA, the fitness of the viruses was first investigated as compared with wild-type virus (data not shown). Only three mutant viruses (K98A, T81A, and R76A) displayed enough infectivity as compared with wild-type virus to be used in this study. Each compound was then tested against the K98A, T81A, and R76A mutants in both the infection and production assays. Each compound was tested against mutants that were proposed to be involved in direct interaction with that compound based on the docking model. As such, compounds CMPD-18, CMPD-7, and CMPD-14 were tested against R76A, T81A, and K98A and CMPD-17 was tested against R76A and K98A. The results of this study (FIG. 15) demonstrated that the MTI compounds act through MA in the context of the virus and interact specifically with residues within a highly conserved binding pocket in MA.

### Example 10

### Specificity for Retroviruses

The specificity analysis for retroviruses was then extended to include other RNA- and DNA-based viruses. To determine the spectrum of antiviral activity of the MTI compounds, CMPD-18, CMPD-17, CMPD-7, and CMPD-14 were evaluated in cellular assays against a panel of viruses from different classes. The compounds were evaluated against this panel of viruses up to a high-test concentration of 100 μM and displayed no inhibitory effects on the replication of herpes simplex type 1, Dengue serotypes 1-4, influenza H1N1, respiratory syncytial virus, yellow fever, Japanese encephalitis, Vaccinia, or Chikungunya viruses. Therefore, the MTI compounds appear to be specific for the related retroviruses HIV-1 and SIV.

### Example 11

### Antiviral Activity Against Multiple Subtypes of HIV-1

An issue in the development of novel HIV drugs is their ability to inhibit the replication of genetically diverse isolates, especially isolates from the most globally prevalent subtypes, A, B, and C. Therefore, the antiviral efficacy of CMPD-18, CMPD-17, CMPD-7, and CMPD-14 was further evaluated in PBMCs against a panel of HIV-1 clinical isolates from different geographic locations that included HIV-1 group M subtypes A, B, C, D, E, F, and G, as well as HIV-1 group O. The panel included CCR5-tropic (R5), CXCR4-tropic (X4), and dual-tropic (R5X4) viruses. As summarized in Table 5, the MTI compounds inhibited the replication of viruses from all group M subtypes (A, B, C, and D, E, F, and G) and from the group O isolate. One exception was CMPD-7, which displayed no activity against the group O isolate HIV-1_{BCF02} over the concentrations tested. Testing results for additional compounds of the invention are summarized in Tables 6 and 7.

TABLE 5
Therapeutic spectrum of the MTI compounds against HIV-1 Subtypes

| | | | | Antiviral |
|---|---|---|---|---|
| HIV-1 | | IC₅₀ | IC₉₀ | index^{†} |
| isolate | Compound | (μM) | (μM) | (TC₅₀/IC₅₀) |
| 92UG031 | CMPD-18 | 15.8 | 64.7 | >3.98 |
| Subtype A | CMPD-14 | 18.5 | 42.2 | >5.40 |
| | CMPD-7 | 14.9 | >100 | >6.72 |
| | CMPD-17 | 53.4 | >100 | >1.87 |
| 89BZ167 | CMPD-18 | 21.3 | 71.8 | >2.94 |
| Subtype B | CMPD-14 | 21.4 | 40.7 | >4.66 |
| | CMPD-7 | 14.4 | >100 | >6.97 |
| | CMPD-17 | 39.8 | >100 | >2.51 |
| 92BR025 | CMPD-18 | 22.4 | 75.0 | >2.79 |
| Subtype C | CMPD-14 | 23.6 | 96.6 | >4.24 |
| | CMPD-7 | 10.5 | >100 | >9.51 |
| | CMPD-17 | 65.0 | >100 | >1.54 |
| 92UG024 | CMPD-18 | 10.4 | 25.9 | >6.02 |
| Subtype D | CMPD-14 | 18.5 | 29.5 | >5.41 |
| | CMPD-7 | 8.88 | 35.2 | >11.3 |
| | CMPD-17 | 25.0 | >100 | >4.01 |
| CMU08 | CMPD-18 | 15.9 | 95.0 | >5.00 |
| Subtype E | CMPD-14 | 16.0 | 59.5 | >6.27 |
| | CMPD-7 | 9.64 | 99.7 | >10.4 |
| | CMPD-17 | 15.5 | >100 | >6.46 |
| 93BR020 | CMPD-18 | 18.5 | 54.1 | >3.39 |
| Subtype F | CMPD-14 | 20.3 | 32.6 | >4.93 |
| | CMPD-7 | 15.6 | >100 | >6.42 |
| | CMPD-17 | 22.9 | >100 | >4.37 |
| G3 | CMPD-18 | 5.73 | 21.0 | >10.9 |
| Subtype G | CMPD-14 | 20.5 | 29.2 | >4.88 |
| | CMPD-7 | 10.7 | 30.2 | >9.36 |
| | CMPD-17 | 23.1 | 94.5 | >4.32 |
| BCF02 | CMPD-18 | 27.2 | 75.6 | >2.30 |
| Group O | CMPD-14 | 21.9 | 45.0 | >4.57 |
| | CMPD-7 | >100 | >100 | NA |
| | CMPD-17 | 94.1 | >100 | >1.06 |

^{†}TC₅₀ values for all compounds were determined to be >100 μM in this study.

TABLE 6
Therapeutic spectrum of the MTI compounds against HIV-1 Subtypes

| | | | | | Antiviral |
|---|---|---|---|---|---|
| | Virus | IC₉₀ | IC₅₀ | TC₅₀ | Index |
| Compound | Isolate | (μM) | (μM) | (μM) | (TC₅₀/IC₅₀) |
| CMPD-14 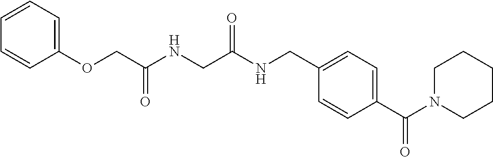 [SMILES: O=C(CNC(=O)COc1ccccc1)NCc1ccc(C(=O)N2CCCCC2)cc1] | Ba-L NL4-3 | >100 >100 | 32.0 39.6 | >100 | >3.13 >2.53 |
| CMPD-35 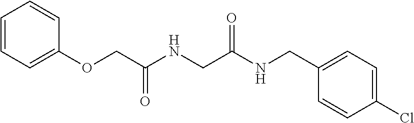 [SMILES: O=C(CNC(=O)COc1ccccc1)NCc1ccc(Cl)cc1] | Ba-L NL4-3 | >100 >100 | >100 >100 | >100 | NA NA |
| CMPD-36 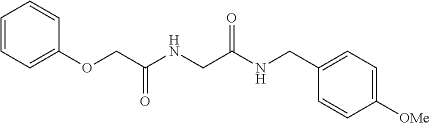 [SMILES: COc1ccc(CNC(=O)CNC(=O)COc2ccccc2)cc1] | Ba-L NL4-3 | >100 >100 | >100 >100 | >100 | NA NA |
| CMPD-37 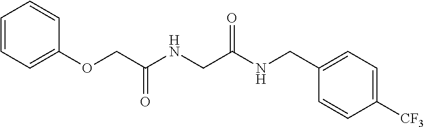 [SMILES: O=C(CNC(=O)COc1ccccc1)NCc1ccc(C(F)(F)F)cc1] | Ba-L NL4-3 | >100 >100 | >100 >100 | >100 | NA NA |
| CMPD-38 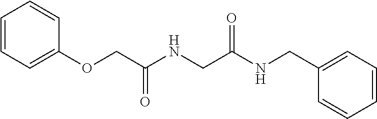 [SMILES: O=C(CNC(=O)COc1ccccc1)NCc1ccccc1] | Ba-L NL4-3 | >100 >100 | >100 >100 | >100 | NA NA |
| CMPD-39 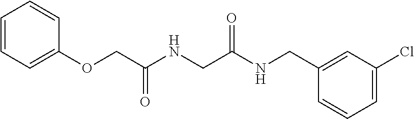 [SMILES: O=C(CNC(=O)COc1ccccc1)NCc1cccc(Cl)c1] | Ba-L NL4-3 | >100 >100 | >100 >100 | >100 | NA NA |
| CMPD-40 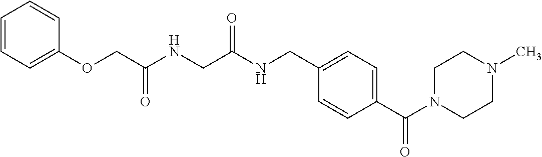 [SMILES: CN1CCN(C(=O)c2ccc(CNC(=O)CNC(=O)COc3ccccc3)cc2)CC1] | Ba-L NL4-3 | >100 >100 | >100 >100 | >100 | NA NA |

TABLE 7
Therapeutic spectrum of the MTI compounds against HIV-1 Subtypes

| | | | | | Anti- |
|---|---|---|---|---|---|
| | | | | | viral |
| | | | | | Index |
| | Virus | IC₉₀ | IC₅₀ | TC₅₀ | (TC₅₀/ |
| Compound | Isolate | (μM) | (μM) | (μM) | IC₅₀) |
| CMPD-41 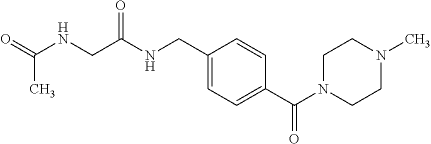 [SMILES: CC(=O)NCC(=O)NCc1ccc(C(=O)N2CCN(C)CC2)cc1] | Ba-L NL4-3 | >100 >100 | >100 >100 | >100 | NA NA |
| CMPD-42 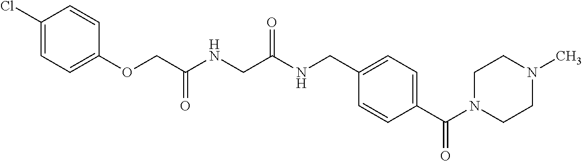 [SMILES: CN1CCN(C(=O)c2ccc(CNC(=O)CNC(=O)COc3ccc(Cl)cc3)cc2)CC1] | Ba-L NL4-3 | >100 >100 | >100 >100 | >100 | NA NA |
| CMPD-43 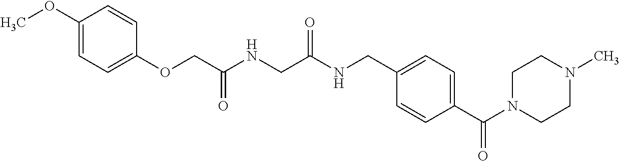 | Ba-L NL4-3 | >100 >100 | >100 >100 | >100 | NA NA |
| CMPD-44 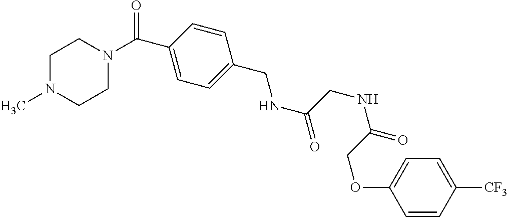 [SMILES: CN1CCN(C(=O)c2ccc(CNC(=O)CNC(=O)COc3ccc(C(F)(F)F)cc3)cc2)CC1] | Ba-L NL4-3 | >100 >100 | >100 >100 | >100 | NA NA |
| CMPD-45 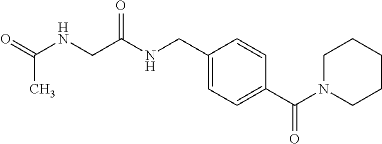 [SMILES: CC(=O)NCC(=O)NCc1ccc(C(=O)N2CCCCC2)cc1] | Ba-L NL4-3 | >100 >100 | >100 >100 | >100 | NA NA |
| CMPD-46 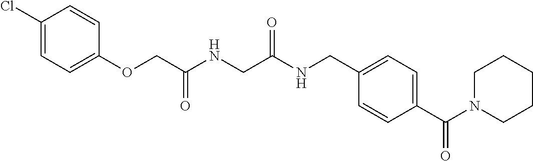 [SMILES: O=C(CNC(=O)COc1ccc(Cl)cc1)NCc1ccc(C(=O)N2CCCCC2)cc1] | Ba-L NL4-3 | >100 >100 | 72.6 78.1 | >100 | >1.38 >1.28 |
| CMPD-47 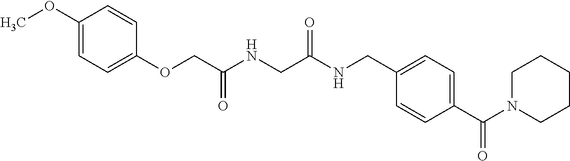 [SMILES: COc1ccc(OCC(=O)NCC(=O)NCc2ccc(C(=O)N3CCCCC3)cc2)cc1] | Ba-L NL4-3 | 96.1 92.7 | 50.8 52.7 | 73.1 | 1.44 1.39 |
| CMPD-48 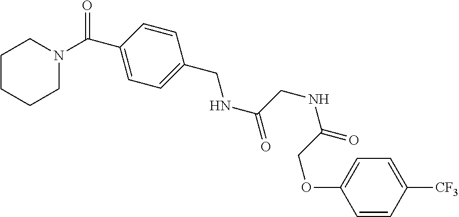 [SMILES: O=C(CNC(=O)COc1ccc(C(F)(F)F)cc1)NCc1ccc(C(=O)N2CCCCC2)cc1] | Ba-L NL4-3 | >100 >100 | 20.6 22.5 | >100 | >4.86 >4.45 |

In the experiments described herein, a structure-based virtual screen was used to identify small molecules capable of disrupting HIV-1 replication by interfering with the normal functions of the MA protein. To perform this screen a newly described virtual screening platform, QUANTUM, was used, applying a sequence of molecular mechanics methods with implicit solvent models to evaluate binding free energies (Joce et al., 2010, Bioorg. Med. Chem. Lett. 20:5411-13). The identification of potential small-molecule MA inhibitors by this method was twofold, combining fast molecular docking for the generation of binding poses and molecular dynamics simulations to rank the ligand poses according to their binding affinities. To limit the amount of necessary calculations to a reasonable level, extensive clustering of the ligand library was performed to reduce the computational burden, while keeping as much of the full chemical diversity of the available library as possible. Using these techniques, a virtual screening library of approximately 1.1 million was narrowed down to 19 compounds that had the potential to bind to MA and disrupt the functions of HIV-1.

These compounds were analyzed for antiviral activity using single-round infection assays, which make use of recombinant viruses, and multiple-round infection assays with primary HIV-1 isolates replicating in PBMCs. Due to the modular nature of the single-round assay, it was possible to determine both whether the compound has antiviral activity and at which stage of replication, early or late, this activity was manifested. However, this assay relied on the use of recombinant virus and immortalized cell lines, which were usually poor representations of the physiological cell targets of HIV-1. Therefore, a secondary screening of the compounds identified within the single-round infection assay was performed using primary isolates replicating in PBMCs. The combined use of these antiviral screens resulted in the identification of four novel chemotypes that display antiviral activity and apparently function by mechanisms that interfere with early, late, or both early and late events in the viral replication cycle. Moreover, the identified inhibitors demonstrated little or no cytotoxicity over the concentration range tested (up to 100 μM); were retroviral specific (showing no inhibition of a panel of nonretroviral DNA and RNA viruses); possessed broad-spectrum anti-HIV activity (Table 5); and worked via interaction with HIV-1 MA (FIGS. 14 and 15).

Herein novel inhibitors that bind to and function through a conserved binding site on HIV-1 MA were identified. These inhibitors inhibited replication of the virus at two points in the replication cycle; and exhibited broad-spectrum anti-HIV activity with IC₅₀ values in the range of 5 to 65 μM for the Group M isolates tested (Table 5). The broad-spectrum activity of these compounds is attractive, highlighting the HIV-1 MA protein as a new viral target with significant therapeutic opportunity. Additional investigations into the mechanism of action of these compounds may shed light on the biological functions of HIV-1 MA within the viral replication cycle.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of inhibiting, suppressing or preventing a retrovirus infection in a subject in need thereof, said method comprising administering to said subject a composition comprising a therapeutically effective amount of at least one compound selected from the group consisting of:
N-benzyl-3-(2-(2-(2,5-dimethoxyphenyl)pyrrolidin-1-yl)-2-oxoethoxy)benzamide (CMPD-1);
2-(2-methyl-6,7-dihydro-1H-[1,4]dioxino[2′,3′:4,5]benzo[1,2-d]imidazol-1-yl)-N-(3-(piperidin-1-ylsulfonyl)phenyl)acetamide (CMPD-2);
(3,5-dichloro-4-methoxyphenyl)(4-((3-(p-tolyl)-1,2,4-oxadiazol-5-yl)methyl)piperazin-1-yl)methanone (CMPD-3);
N-(4-(cyclopropanecarboxamido)benzyl)-4-(2-(4-methoxyphenoxy)ethyl)piperazine-1-carboxamide (CMPD-4);
1-(2,3-dihydrobenzo[b][1,4]dioxin-6-yl)-N-((1-methyl-1H-benzo[d]imidazol-2-yl)methyl)-5-oxopyrrolidine-3-carboxamide (CMPD-5);
2-(4-(3-(benzo[d]oxazol-2-yl)propanoyl)piperazin-1-yl)-N-(4-(trifluoromethoxy)phenyl)acetamide (CMPD-9);
N-(5-acetamido-2-methoxyphenyl)-2-(5-(5-methylthiophen-2-yl)-4-oxothieno[2,3-d]pyrimidin-3(4H)-yl)acetamide (CMPD-10);
N-(benzylcarbamoyl)-2-(4-(2-oxo-2-(pyrrolidin-1-yl)ethyl)piperazin-1-yl)acetamide (CMPD-1);
2-(2-oxo-2-((4-(pyrrolidin-1-yl)phenyl)amino)ethoxy)-N-phenylbenzamide (CMPD-15);
N-(4-((4-bromophenyl)thio)phenyl)-2-(4-oxoquinazolin-3(4H)-yl)acetamide (CMPD-16);
4-(1,3-dithian-2-yl)-N-(4-(pyrrolidin-1-ylmethyl)benzyl)benzamide (CMPD-19);
2-acetamido-N-(4-(piperidine-1-carbonyl)benzyl)acetamide (CMPD-45);
a compound of Formula (I);
a compound of Formula (II);
a compound of Formula (III);
a compound of Formula (IV);
a compound of Formula (V);
a compound of Formula (VI);
a compound of Formula (VII);
a compound of Formula (VIII);
mixtures thereof and pharmaceutically acceptable carriers thereof, wherein:
said compound of Formula (I) is: 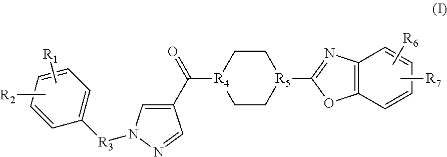
wherein in Formula (I):
R₁, R₂, R₆ and R₇ are independently H, fluoro, chloro, bromo, C₁-C₆ alkyl, or C₁-C₆ alkoxy,
R₃ is CH₂ or a chemical bond, and
R₄ and R₅ are independently CH or N,
or a pharmaceutically acceptable salt thereof;
said compound of Formula (II) is: 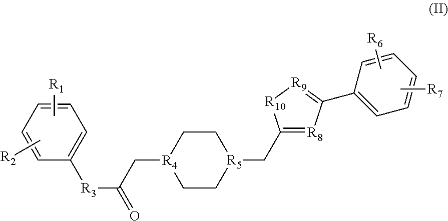
wherein in Formula (II):
R₁, R₂, R₆ and R₇ are independently H, fluoro, chloro, bromo, —CF₃, C₁-C₆ alkyl, or C₁-C₆ alkoxy,
R₃ is NH, CH₂ or a chemical bond,
R₄ and R₅ are independently CH or N,
R₈ and R₉ are independently CH or N, and
R₁₀ is O or S;
or a pharmaceutically acceptable salt thereof;
said compound of Formula (III) is: 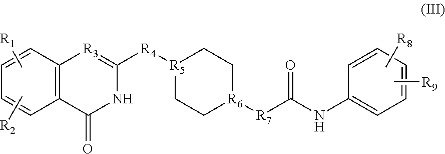
wherein in Formula (III):
R₁, R₂, R₈ and R₉ are independently H, fluoro, chloro, bromo, C₁-C₆ alkyl, or C₁-C₆ alkoxy,
R₃ is CH or N,
R₄ and R₇ are independently CH₂ or a chemical bond, and
R₅ and R₆ are independently CH or N;
or a pharmaceutically acceptable salt thereof;
said compound of Formula (IV) is: 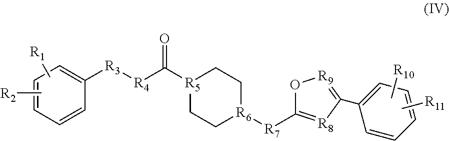
wherein in Formula (IV):
R₁, R₂, R₁₀ and R₁₁ are independently H, fluoro, chloro, bromo, C₁-C₆ alkyl, or C₁-C₆ alkoxy,
R₃ is O or NH,
R₄ and R₇ are independently CH₂ or a chemical bond, and
R₅, R₆, R₈ and R₉ are independently CH or N;
or a pharmaceutically acceptable salt thereof;
said compound of Formula (V) is: 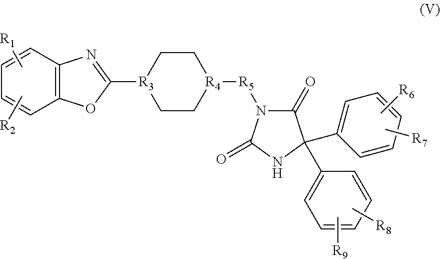
wherein in Formula (V):
R₁, R₂, R₆, R₇, R₈ and R₉ are independently H, fluoro, chloro, bromo, C₁-C₆ alkyl, or C₁-C₆ alkoxy,
R₃ are R₄ are independently CH or N, and
R₅ is CH₂ or a chemical bond;
or a pharmaceutically acceptable salt thereof;
said compound of Formula (VI) is: 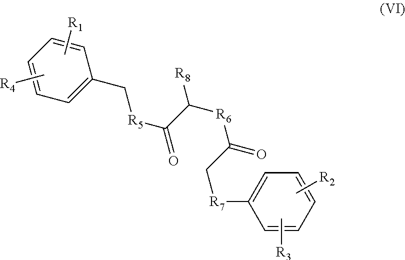
wherein in Formula (VI):
R₁, R₂, and R₃ are independently H, fluoro, chloro, bromo, C₁-C₆ alkyl, or C₁-C₆ alkoxy,
R₄ is H, halo, C₁-C₆ alkyl, C₁-C₆ alkoxy, —SO₂NH₂, —NH₂, —NHC(═O)CH₃, —CF₃, or —C(═O)R₉,
R₅, R₆ and R₇ are independently a chemical bond, O, CH₂ or NH,
R₈ is H or C₁-C₆ alkyl,
R₉ is H, C₁-C₆ alkyl, N,N-dimethylamino, N,N-diethylamino, N-morpholinyl, N-pyrrolidinyl, N-piperidinyl, N-piperazinyl, or N′—(C₁-C₆ alkyl)piperazinyl,
or a pharmaceutically acceptable salt thereof;
said compound of Formula (VII) is: 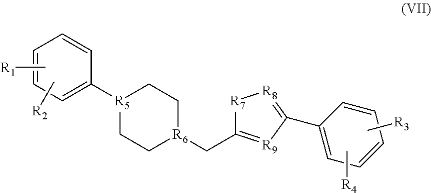
wherein in Formula (VII):
R₁, R₂, R₃, and R₄ are independently H, nitro, amino, fluoro, chloro, bromo, C₁-C₆ alkyl, or C₁-C₆ alkoxy,
R₅, R₆, R₈ and R₉ are independently CH or N, and
R₇ is S or O,
or a pharmaceutically acceptable salt thereof;
and said compound of Formula (VIII) is: 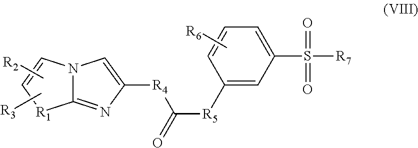
wherein in Formula (VIII):
R₁ is O, S or —CH═CH—;
R₂, R₃, and R₆ are independently H, fluoro, chloro, bromo, C₁-C₆ alkyl, or C₁-C₆ alkoxy,
R₄ and R₅ are independently CH₂ or NH, and
R₇ is C₁-C₆ alkyl, N,N-dimethylamino, N,N-diethylamino, N-morpholinyl, N-pyrrolidinyl or N-piperidinyl,
or a pharmaceutically acceptable salt thereof.

2. The method of claim 1, wherein said at least one compound is selected from the group consisting of:
N-benzyl-3-(2-(2-(2,5-dimethoxyphenyl)pyrrolidin-1-yl)-2-oxoethoxy)benzamide (CMPD-1);
2-(2-methyl-6,7-dihydro-1H-[1,4]dioxino[2′,3′:4,5]benzo[1,2-d]imidazol-1-yl)-N-(3-(piperidin-1-ylsulfonyl)phenyl)acetamide (CMPD-2);
(3,5-dichloro-4-methoxyphenyl)(4-((3-(p-tolyl)-1,2,4-oxadiazol-5-yl)methyl)piperazin-1-yl)methanone (CMPD-3);
N-(4-(cyclopropanecarboxamido)benzyl)-4-(2-(4-methoxyphenoxy)ethyl)piperazine-1-carboxamide (CMPD-4);
1-(2,3-dihydrobenzo[b][1,4]dioxin-6-yl)-N-((1-methyl-1H-benzo[d]imidazol-2-yl)methyl)-5-oxopyrrolidine-3-carboxamide (CMPD-5);
(4-(benzo[d]oxazol-2-yl)piperidin-1-yl)(1-(2-chlorobenzyl)-1H-pyrazol-4-yl)methanone (CMPD-6);
2-(4-((3-(4-fluorophenyl)-1,2,4-oxadiazol-5-yl)methyl)piperazin-1-yl)-N-(p-tolyl)acetamide (CMPD-7);
2-(4-((6,7-dimethoxy-4-oxo-3,4-dihydroquinazolin-2-yl)methyl)piperazin-1-yl)-N-(2,3-dimethylphenyl)acetamide (CMPD-8);
2-(4-(3-(benzo[d]oxazol-2-yl)propanoyl)piperazin-1-yl)-N-(4-(trifluoromethoxy)phenyl)acetamide (CMPD-9);
N-(5-acetamido-2-methoxyphenyl)-2-(5-(5-methylthiophen-2-yl)-4-oxothieno[2,3-d]pyrimidin-3(4H)-yl)acetamide (CMPD-10);
N-(benzylcarbamoyl)-2-(4-(2-oxo-2-(pyrrolidin-1-yl)ethyl)piperazin-1-yl)acetamide (CMPD-11);
2-(2,5-dichlorophenoxy)-1-(4-((3-(4-methoxyphenyl)-1,2,4-oxadiazol-5-yl)methyl)piperazin-1-yl)ethanone (CMPD-12);
3-((4-(benzo[d]oxazol-2-yl)piperidin-1-yl)methyl)-5,5-diphenylimidazolidine-2,4-dione (CMPD-13);
N-(2-oxo-2-((4-(piperidine-1-carbonyl)benzyl)amino)ethyl)-2-phenoxyacetamide (CMPD-14);
2-(2-oxo-2-((4-(pyrrolidin-1-yl)phenyl)amino)ethoxy)-N-phenylbenzamide (CMPD-15);
N-(4-((4-bromophenyl)thio)phenyl)-2-(4-oxoquinazolin-3(4H)-yl)acetamide (CMPD-16);
3-(2-ethoxyphenyl)-5-((4-(4-nitrophenyl)piperazin-1-yl)methyl)-1,2,4-oxadiazole (CMPD-17);
N-(4-ethoxy-3-(piperidin-1-ylsulfonyl)phenyl)-2-(imidazo[2,1-b]thiazol-6-yl)acetamide (CMPD-18);
4-(1,3-dithian-2-yl)-N-(4-(pyrrolidin-1-ylmethyl)benzyl)benzamide (CMPD-19);
N-(4-fluorophenyl)-2-(4-((3-(4-fluorophenyl)-1,2,4-oxadiazol-5-yl)methyl)piperazin-1-yl)acetamide (CMPD-20);
N-(2,3-dimethylphenyl)-2-(4-((3-(4-fluorophenyl)-1,2,4-oxadiazol-5-yl)methyl)piperazin-1-yl)acetamide (CMPD-21);
N-(2-chlorophenyl)-2-(4-((3-(4-fluorophenyl)-1,2,4-oxadiazol-5-yl)methyl)piperazin-1-yl)acetamide (CMPD-22);
N-(2,6-dimethylphenyl)-2-(4-((3-(4-fluorophenyl)-1,2,4-oxadiazol-5-yl)methyl)piperazin-1-yl)acetamide (CMPD-23);
N-(3-fluorophenyl)-2-(4-((3-(4-fluorophenyl)-1,2,4-oxadiazol-5-yl)methyl)piperazin-1-yl)acetamide (CMPD-24);
N-(2-ethylphenyl)-2-(4-((3-(4-fluorophenyl)-1,2,4-oxadiazol-5-yl)methyl)piperazin-1-yl)acetamide (CMPD-25);
2-(4-((3-(4-fluorophenyl)-1,2,4-oxadiazol-5-yl)methyl)piperazin-1-yl)-N-(2-(trifluoromethyl)phenyl)acetamide (CMPD-26);
2-(2-phenoxyacetamido)-N-(3-(trifluoromethyl)benzyl)propanamide (CMPD-27);
2-(2-phenoxyacetamido)-N-(4-sulfamoylbenzyl)propanamide (CMPD-28), 2-(2-phenoxyacetamido)-N-(4-methoxybenzyl)propanamide (CMPD-29);
3-methyl-N-(4-methylbenzyl)-2-(2-phenoxyacetamido)butanamide (CMPD-30);
N-benzyl-3-methyl-2-(2-phenoxyacetamido)butanamide (CMPD-32);
N-(4-acetamidobenzyl)-2-(2-phenoxyacetamido)propanamide (CMPD-33);
N-(3-acetamidobenzyl)-2-(2-phenoxyacetamido)acetamide (CMPD-34);
N-(4-chlorobenzyl)-2-(2-phenoxyacetamido)acetamide (CMPD-35);
N-(4-methoxybenzyl)-2-(2-phenoxyacetamido)acetamide (CMPD-36);
N-(2-oxo-2-((4-(trifluoromethyl)benzyl)amino)ethyl)-2-phenoxyacetamide (CMPD-37);
N-benzyl-2-(2-phenoxyacetamido)acetamide (CMPD-38);
N-(3-chlorobenzyl)-2-(2-phenoxyacetamido)acetamide (CMPD-39);
N-(4-(4-methylpiperazine-1-carbonyl)benzyl)-2-(2-phenoxyacetamido) acetamide (CMPD-40);
2-acetamido-N-(4-(4-methylpiperazine-1-carbonyl)benzyl)acetamide (CMPD-41);
2-(4-chlorophenoxy)-N-(2-((4-(4-methylpiperazine-1-carbonyl)benzyl)amino)-2-oxoethyl)acetamide (CMPD-42);
2-(4-methoxyphenoxy)-N-(2-((4-(4-methylpiperazine-1-carbonyl)benzyl)amino)-2-oxoethyl)acetamide (CMPD-43);
N-(4-(4-methylpiperazine-1-carbonyl)benzyl)-2-(2-(4-(trifluoromethyl)phenoxy)acetamido)acetamide (CMPD-44);
2-acetamido-N-(4-(piperidine-1-carbonyl)benzyl)acetamide (CMPD-45);
2-(4-chlorophenoxy)-N-(2-oxo-2-((4-(piperidine-1-carbonyl)benzyl)amino)ethyl)acetamide (CMPD-46);
2-(4-methoxyphenoxy)-N-(2-oxo-2-((4-(piperidine-1-carbonyl)benzyl)amino)ethyl)acetamide (CMPD-47);
N-(2-oxo-2-((4-(piperidine-1-carbonyl)benzyl)amino)ethyl)-2-(4-(trifluoromethyl)phenoxy)acetamide (CMPD-48);
3-(4-fluorophenyl)-5-((4-(2-methoxyphenyl)piperazin-1-yl)methyl)-1,2,4-oxadiazole (CMPD-49);
3-(4-fluorophenyl)-5-((4-(4-methoxyphenyl)piperazin-1-yl)methyl)-1,2,4-oxadiazole (CMPD-50);
3-(2-ethoxyphenyl)-5-((4-(4-nitrophenyl)piperazin-1-yl)methyl)-1,2,4-oxadiazole (CMPD-51);
3-(4-fluorophenyl)-5-((4-phenylpiperazin-1-yl)methyl)-1,2,4-oxadiazole (CMPD-52);
a mixture thereof and a pharmaceutically acceptable carrier thereof.

3. The method of claim 2, wherein said at least one compound is selected from the group consisting of:
(4-(benzo[d]oxazol-2-yl)piperidin-1-yl)(1-(2-chlorobenzyl)-1H-pyrazol-4-yl)methanone (CMPD-6);
2-(4-((3-(4-fluorophenyl)-1,2,4-oxadiazol-5-yl)methyl)piperazin-1-yl)-N-(p-tolyl)acetamide (CMPD-7);
2-(4-((6,7-dimethoxy-4-oxo-3,4-dihydroquinazolin-2-yl)methyl)piperazin-1-yl)-N-(2,3-dimethylphenyl)acetamide (CMPD-8);
2-(2,5-dichlorophenoxy)-1-(4-((3-(4-methoxyphenyl)-1,2,4-oxadiazol-5-yl)methyl)piperazin-1-yl)ethanone (CMPD-12);
3-((4-(benzo[d]oxazol-2-yl)piperidin-1-yl)methyl)-5,5-diphenyl imidazolidine-2,4-dione (CMPD-13);
N-(2-oxo-2-((4-(piperidine-1-carbonyl)benzyl)amino)ethyl)-2-phenoxyacetamide (CMPD-14);
3-(2-ethoxyphenyl)-5-((4-(4-nitrophenyl)piperazin-1-yl)methyl)-1,2,4-oxadiazole (CMPD-17);
N-(4-ethoxy-3-(piperidin-1-ylsulfonyl)phenyl)-2-(imidazo[2,1-b]-thiazol-6-yl)acetamide (CMPD-18);
a mixture thereof and a pharmaceutically acceptable carrier thereof.

4. The method of claim 3, wherein said at least one compound is (4-(benzo[d]oxazol-2-yl)piperidin-1-yl)(1-(2-chlorobenzyl)-1H-pyrazol-4-yl)methanone (CMPD-6), or a pharmaceutically acceptable carrier thereof.

5. The method of claim 3, wherein said at least one compound is 2-(4-((3-(4-fluorophenyl)-1,2,4-oxadiazol-5-yl)methyl)piperazin-1-yl)-N-(p-tolyl)acetamide (CMPD-7), or a pharmaceutically acceptable carrier thereof.

6. The method of claim 3, wherein said at least one compound is 2-(4-((6,7-dimethoxy-4-oxo-3,4-dihydroquinazolin-2-yl)methyl)piperazin-1-yl)-N-(2,3-dimethylphenyl)acetamide (CMPD-8), or a pharmaceutically acceptable carrier thereof.

7. The method of claim 3, wherein said at least one compound is 2-(2,5-dichlorophenoxy)-1-(4-((3-(4-methoxyphenyl)-1,2,4-oxadiazol-5-yl)methyl)piperazin-1-yl)ethanone (CMPD-12), or a pharmaceutically acceptable carrier thereof.

8. The method of claim 3, wherein said at least one compound is 3-((4-(benzo[d]oxazol-2-yl)piperidin-1-yl)methy)-5,5-diphenylimidazolidine-2,4-dione (CMPD-13), or a pharmaceutically acceptable carrier thereof.

9. The method of claim 3, wherein said at least one compound is N-(2-oxo-2-((4-(piperidine-1-carbonyl)benzyl)amino)ethyl)-2-phenoxyacetamide (CMPD-14), or a pharmaceutically acceptable carrier thereof.

10. The method of claim 3, wherein said at least one compound is 3-(2-ethoxyphenyl)-5-((4-(4-nitrophenyl)piperazin-1-yl)methyl)-1,2,4-oxadiazole (CMPD-17), or a pharmaceutically acceptable carrier thereof.

11. The method of claim 3, wherein said at least one compound is N-(4-ethoxy-3-(piperidin-1-ylsulfonyl)phenyl)-2-(imidazo[2,1-b]thiazol-6-yl)acetamide (CMPD-18), or a pharmaceutically acceptable carrier thereof.

12. The method of claim 1, wherein said retrovirus is selected from the group consisting of ALV, RSV, MMTV, SRV, HERV-K, JRSV, MLV, FeLV, GALV, PERV, HERV-W, BLV, HTLV-I, HTLV-II, WDSV, SnRV, HIV-1, HIV-2, SIVmac, SIV, FIV, EIAV, MVV, SFVcpz, SFVagm, FFV, BFV and combinations thereof.

13. The method of claim 12, wherein said retrovirus is selected from the group consisting of MLV, HIV-1, SIV, and combinations thereof.

14. The method of claim 1, wherein said composition further comprises one or more anti-HIV drugs.

15. The method of claim 14, wherein said one or more anti-HIV drugs are selected from the group consisting of combination drugs, entry and fusion inhibitors, integrase inhibitors, non-nucleoside reverse transcriptase inhibitors, nucleoside reverse transcriptase inhibitors, and protease inhibitors.

16. The method of claim 1, wherein said subject is a mammal.

17. The method of claim 1, wherein said subject is human.


SEQUENCE LISTING (3 sequences)

SEQ ID NO: 1 - protein, 238 aa, Human immunodeficiency virus
MGHHHHHHGGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSSQVSQNY

SEQ ID NO: 2 - protein, 101 aa, Artificial Sequence
  Chemically Synthesized
MSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 3 - protein, 14 aa, Artificial Sequence
  Chemically Synthesized
GLNDIFEAQKIEWH